(19) United States
(12) Patent Application Publication
(10) Pub. No.: US 2025/0340603 A1
(43) Pub. Date: Nov. 6, 2025

(54) A METHOD FOR MODULATING PLANT ADAPTATION TRAITS

(71) Applicant: CRAG (Centre de Recerca en Agrigenòmica), Barcelona (ES)

(72) Inventors: Ana Isabel Caño-Delgado, Barcelona (ES); Aditi Gupta, Barcelona (ES); Fidel Lozano-Elena, Barcelona (ES); Andrés RICO-MEDINA, Barcelona (ES)

(73) Assignee: CRAG (Centre de Recerca en Agrigenòmica), Barcelona (ES)

(21) Appl. No.: 18/565,555

(22) PCT Filed: Jun. 2, 2022
(86) PCT No.: PCT/EP2022/065072

§ 371 Date: Jun. 21, 2024

(30) Foreign Application Priority Data
Jun. 3, 2021 (EP) 21382498.0

(51) Int. Cl. C07K 14/415; C12N 15/82

(52) U.S. Cl. CPC C07K 14/415; C12N 15/8222

(57) ABSTRACT

The current invention relates to the field of plant biology, breeding and agriculture. The invention relates to methods of generating a plant comprising expressing the BRL3 pathway in phloem tissues of said plant.

### SEQUENCE LISTING

The instant application contains a Sequence Listing which has been submitted electronically in txt file format and is hereby incorporated by reference in its entirety. Said txt copy, created on Jun. 20, 2024, is named P6100130PCT-US_SEQ listing.txt and is 149 KB in size.

### FIELD OF THE INVENTION

The current invention relates to the field of plant biology, breeding and agriculture. The invention relates to methods of generating a plant comprising expressing the BRL3 pathway in phloem tissues of said plant.

### BACKGROUND OF THE INVENTION

Plant steroid hormones were named brassinosteroids (BRs) since they were discovered in the pollen of the plant Brassica napus, where they are very abundant. BRs are perceived at cell's plasma membrane by an Leucine-Rich Repeat Recepor-Like Kinase (LRR-RLK) named BRASSINOSTEROID INSENSITIVE 1 (BRI1).

In Arabidopsis, there are two closely related members of the small BRI1-like family, BRASSINOSTEROID RECEPTOR UKE 1 and 3 (BRL1 and BRL3 respectively), that share the overall structure of BRI1, including the ligand-binding ID, and can bind to BL with higher (BRL1) or similar (BRL3) binding affinity as the main BRI1 receptor (Caño-Delgado et al., 2004; Kinoshita et al., 2005). While BRI1 is expressed in most if not all cells (Friedrichsen et al., 2000), the expression of BRL1 and BRL3 is enriched in the vascular tissues. The analysis of the bri1 brl1 brl3 mutant in the inflorescence stem suggested a redundant role with BRI1 for these two receptors in regulating cell proliferation during vascular bundle patterning (Caño-Delgado et al., 2004). Since then, the discrete localization of BRLs together with the dramatic phenotype of triple BR-receptor mutants has hampered the identification of novel specific roles for BRL receptors in plant growth and development. Recently, the analysis of the BRL3 receptor complex composition in vivo showed that BRL3 physically interacts with the BRL1 receptor and the BAK1 co-receptor, and reveals that these protein receptor interactions contribute to root growth and development in Arabidopsis (Fàbregas et al., 2013).

Some plant mutants in the BR pathway have already been generated. Caño-Delgado et al., 2004 describes a triple mutant (bri1-104 brl1brl3) concerning the BRI1, BRL1 and BRL3 genes, however this plant demonstrates a dramatic phenotype (extreme dwarfism).

Climate change is leading us toward a warmer, drier world (Gupta et al., 2020). Our planet has been consistently getting warmer since the late 18th century, which appears to be accelerating in the recent years at alarming rates. In past decade, all regions across the world had a 10-year average annual temperature change of at least 1.0° C. with Europe leading with an average annual temperature change of 2.1° C. in 2019 (FAO 2020). This climate change trend predicts the global atmospheric temperature to rise by approximately 4° C. by 2080. Rising temperatures can radically hamper food production. Higher temperature is also considered an accomplice in pests and diseases outbreaks in plants and animals alike (Peace, 2020).

Heat stress will become more common as a result of global warming. Therefore, an increasing interest and a need exists for generating crops resistant to abiotic stresses such as heat stress.

### DETAILED DESCRIPTION

### Method

In a first aspect, the current invention provides a method for modulating a plant adaptation trait, the method comprising expressing the BRL3 pathway (preferably overexpressing) in the phloem of said plant, preferably wherein the gene BRL3 is expressed (preferably overexpressed) in the phloem.

In an embodiment, the invention comprises a method for modulating a plant adaptation trait, the method comprising specifically expressing the BRL3 pathway in the phloem of said plant, preferably wherein the gene BRL3 is expressed in the phloem of said plant.

As defined herein, “the phloem” may be considered as a tissue or an organ of a plant. In an embodiment, the phloem is a vascular tissue. The phloem may be present in several organs of a plant such as are roots, stems, stalks, leaves, petals, fruits, seeds, tubers, pollen, meristems, callus, sepals, bulbs and flowers.

As defined herein, “specifically expressing the BRL3 pathway in the phloem” of a plant may be considered as expression of the BRL3 pathway in the phloem and in a maximum number of three other plant tissues of said plant. Specific expression in the phloem of a plant may primarily result in expression of the BRL3 pathway in the phloem, but can also result in detectable level (“leaky”) of the BRL3 pathway expression in other plant tissues. This leaky expression of the BRL3 pathway in other plant tissues may be at a lower detectable expression as compared to the phloem-specific BRL3 pathway expression, as evaluated on the level of the mRNA or the protein by standard assays known to a person of skill in the art (e.g. PCR or Western blot analysis). The maximum number of plant tissues where leaky BRL3 pathway expression may be detected is one, two or three. Accordingly, in one embodiment, the BRL3 pathway is expressed in the phloem and in one other plant tissue. In another embodiment, the BRL3 pathway is expressed in the phloem and in two other plant tissues. In another embodiment, the BRL3 pathway is expressed in the phloem and in three other plant tissues. In a preferred embodiment, the BRL3 pathway expression in restricted to the phloem only. Therefore, the expression “specifically expressing the BRL3 pathway in the phloem” of a plant may be replaced by “expressing the BRL3 pathway in the phloem and in one, two or at the maximum three other tissues of said plant. In a preferred embodiment, the expression” “specifically expressing the BRL3 pathway in the phloem” of a plant may be replaced by “expressing the BRL3 pathway in the phloem and the expression of the BRL3 is not detectable in another tissue of the plant”. The “one or two or three” other plant tissues in which the expression the BRL3 pathway may be detected are preferably vascular tissues of the plant or belong to the vasculature of the plant. Examples of such vascular tissues of the plant include the xylem or the procambium.

Within the context of the invention, “specifically expressing the BRL3 pathway in the phloem” may encompass expression of the BRL3 pathway in phloem companion cells (also called companion cells). Phloem companion cells belong to the phloem tissue.

In an embodiment, the BRL3 pathway, preferably the BRL3 gene is overexpressed in the phloem of a plant.

Within the context of the application, “a plant adaption trait” or “a plant trait” is defined as at least one of the following: the growth physiology, the tolerance to an abiotic stress, a vascular transport and/or the defense response.

In an embodiment of the method of the invention the plant exhibits at least one of the features (or all features) within a), b), c) and/or d):
a) the growth physiology has been modulated: the plant has an increased hypocotyl growth, an increased root growth, an increased petiole length, an altered flowering time and/or a modulation of a marker gene involved in phytohormone response,
b) the tolerance of the plant to an abiotic stress (preferably heat stress) has been improved: the plant has an improved root hydrotropism, an enhanced programmed cell death in root meristems under osmotic stress, a greater hypocotyl growth under heat stress, a greater petiole elongation under heat stress, an improved plant survival rate under heat stress and/or a capacity to accumulate osmoprotectant metabolites in normal conditions and under heat stress;
c) the vascular transport properties of the plant has been improved: the plant exhibits an increased accumulation of metabolites in the roots and/or a modulation of a marker gene for vascular transporter,
d) the defense response of the plant has been modulated,
wherein the modulation of a trait as defined above is by comparison of the same trait in a plant, wherein the BRL3 pathway is not expressed, preferably is not expressed in the phloem, more preferably is not specifically expressed in the phloem. Such a control or reference plant is further defined below.

In an embodiment of the method of the invention the plant exhibits at least one of the features (or all features) within a), b), c) and/or d):
a) the growth physiology has been modulated: the plant has an increased hypocotyl growth, an increased root growth, an increased petiole length, an altered flowering time and/or a modulation of a marker gene involved in phytohormone response,
b) the tolerance of the plant to an abiotic stress (preferably heat stress) has been improved: the plant has an improved root hydrotropism, an enhanced programmed cell death in root meristems under osmotic stress, a greater hypocotyl growth under heat stress, a greater petiole elongation under heat stress, an improved plant survival rate under heat stress, an improved carbon utilization and/or reduced or repressed photorespiration pathway signaling and/or a capacity to accumulate metabolites in normal conditions and under heat stress;
c) the vascular transport properties of the plant has been improved: the plant exhibits an increased accumulation of metabolites in the roots, a decreased phloem unloading in the roots and/or a modulation of a marker gene for vascular transporter,
d) the defense response of the plant has been modulated,
wherein the modulation of a trait as defined above is by comparison of the same trait in a plant, wherein the BRL3 pathway is not expressed, preferably is not expressed in the phloem, more preferably is not specifically expressed in the phloem. Such a control or reference plant is further defined below.

A control or reference plant in this specification may be meant to comprise a plant wherein the expression of BRL3 may not be detected or may not be detectable, otherwise the control or reference plant is similar or identical to the plant as envisioned by the invention.

In an embodiment, a control or reference plant in this specification may be meant to comprise a plant wherein the expression of BRL3 may not be detected in the phloem of said plant or may not be detectable therein, otherwise the control or reference plant is similar or identical to the plant as envisioned by the invention. The words “control” and “reference” in the context of a plant in this specification could be used interchangeably and are regarded in the context of this specification regarded as synonyms.

A control plant in this specification may also be a plant known to the person skilled in the art to be sensitive to an abiotic stress such as heat stress. Typically, a heat stress sensitive plant has a decreased survival rate, a reduced fertility and/or yield loss of at least 5%, 10%, 15%, 20%, 30%, 40%, 50%, 60%, 70%, 80%, or at least 90% or more during heat stress or after a heat stress period when compared to the survival rate, fertility and/or yield of said plant when not exposed to heat stress. In an embodiment, a heat stress sensitive plant may not exhibit a growth adaptation in terms of hypocotyl elongation and/or petiole elongation as the plant of the invention exhibits. An example of a heat stress sensitive plant is Arabidopsis thaliana Columbia (Col-0 ecotype; N1092: NASC stock number). The downregulation of the BRL3 pathway in the control or reference plant has been explained later herein.

“Heat stress” and “elevated temperature” are synonymous in the context of the application. “Heat stress” or “elevated temperature”, for the purpose of this invention, is an extended period of time of at least 1, 2, 3, 4, 5, 6, 7, 8, 9, 10, 11, 12, or 13 days or 2, 3, 4, 5, 6, 7, 8, 9, 10, 11, 12, 13, 14, 15, 16 weeks or longer wherein the temperature is higher than the temperature under normal or average conditions. The temperature under normal or average conditions may also be called the optimum growth temperature. This is the temperature at which a plant will grow the best. The temperature is higher than the temperature under normal or average conditions when it is at least 1 degree Celsius higher than the optimum growth temperature, or it is at least 2 degree Celsius higher than the optimum growth temperature, or it is at least 3 degree Celsius higher than the optimum growth temperature, or it is at least 4 degree Celsius higher than the optimum growth temperature, or it is at least 5 degree Celsius higher than the optimum growth temperature, or it is at least 6, 7, 8, 9, 10, 12, 14, 15 degree Celsius higher than the optimum growth temperature. However, in an embodiment, this temperature is not 10 or 16 degree Celsius higher than the optimum growth temperature.

In an embodiment, heat stress or elevated temperature is at least 1 to at least 6 degree Celsius higher than the optimum growth temperature during a period 1-10 days or 5-15 days.

In an embodiment, heat stress or elevated temperature is at least 1 degree Celsius higher than the optimum growth temperature during a period 1-10 days or 5-15 days.

In an embodiment, heat stress or elevated temperature is at least 2 degree Celsius higher than the optimum growth temperature during a period 1-10 days or 5-15 days.

In an embodiment, heat stress or elevated temperature is at least 3 degree Celsius higher than the optimum growth temperature during a period 1-10 days or 5-15 days.

In an embodiment, heat stress or elevated temperature is at least 4 degree Celsius higher than the optimum growth temperature during a period 1-10 days or 5-15 days.

In an embodiment, heat stress or elevated temperature is at least 5 degree Celsius higher than the optimum growth temperature during a period 1-10 days or 5-15 days.

In an embodiment, heat stress or elevated temperature is at least 6 degree Celsius higher than the optimum growth temperature during a period 1-10 days or 5-15 days.

In an embodiment, heat stress or elevated temperature is at least 8 degree Celsius higher than the optimum growth temperature during a period 1-10 days or 5-15 days.

In an embodiment, heat stress or elevated temperature is at least 10 degree Celsius higher than the optimum growth temperature during a period 1-10 days or 5-15 days.

In an embodiment, heat stress or elevated temperature is at least 12 degree Celsius higher than the optimum growth temperature during a period 1-10 days or 5-15 days.

In an embodiment, heat stress or elevated temperature is at least 14 degree Celsius higher than the optimum growth temperature during a period 1-10 days or 5-15 days.

In an embodiment, heat stress or elevated temperature is at least 15 degree Celsius higher than the optimum growth temperature during a period 1-10 days or 5-15 days.

Normal or average conditions may refer to temperature conditions experienced under the current climate where the plant is cultivated or under controlled conditions in a controlled environment inked to the cultivation. In another embodiment, normal or average conditions may be replaced by optimum growth temperature. Such optimum temperature is different for each plant species/variety. Obviously, “heat stress” is plant species specific and one plant species may experience a “heat stress” at a given temperature while another plant species will not experience such a “heat stress” at the same temperature. This is common knowledge for the skilled person.

As soon as a plant (or plant species) has a decreased survival rate, reduced fertility and/or yield loss of at least 5%, 10%, 15%, 20%, 30%, 40%, 50%, 60%, 70%, 80%, or at least 90% or more after a period of from 1-15 days during which said period the temperature is at least 1 to at least 15 degree Celsius higher than the optimum growth optimum growth temperature, one can say that this plant is sensitive for heat stress. Such a plant may be used as a control or reference plant under these conditions.

A plant of the invention is resistant or tolerant to heat stress. Such a plant has an improved survival rate, fertility and/or yield compared to the one of the control plant as defined in the previous paragraph assessed under the same “heat stress” conditions. The improvement is of at least %, 10%, 15%, 20%, 30%, 40%, 50%, 60%, 70%, 80%, or at least 90% or more measured during the same period of time and under the same stress conditions.

This is also common knowledge that the optimum growth temperature depends of the place on earth where the plant is cultivated. For the purpose of the invention, an adult plant is defined as a plant of at least 3 weeks old. The person skilled in the art will be aware that the minimal age to reach adulthood may differ depending on the plant species.

### Growth Physiology (a)

In an embodiment, under a), the growth physiology of the plant has been modulated: the plant may have an increased hypocotyl growth, an increased root growth, an increased petiole length, an altered flowering time and/or a modulation of a marker gene involved in phytohormone response. Preferably, the growth physiology of the plant has been modulated so that the plant has an increased hypocotyl growth.

A plant of the invention resistant or tolerant to heat stress may have its growth physiology as under a).

Within the context of this invention, a plant with an increased hypocotyl growth is defined as a plant with an increase in average or mean hypocotyl growth. Preferably, the hypocotyl growth of said plant is increased by at least 5%, 10%, 15%, 20%, 25%, 30%, 40%, 50%, 60%, 70%, 80%, 90% or higher when compared to the corresponding percentage of a control or reference plant under the same conditions. Hypocotyl growth may be assessed using techniques known to the skilled person, such as in the experimental part. For hypocotyl elongation analysis, seedlings may be grown in horizontally placed 0.5MS− media plates in controlled growth conditions (Aralab 600; long days 16:8 h day/night cycle 60% relative humidity and 50-70 μmol m⁻² s⁻¹ of cool-white fluorescent light) at 22° C. or 28° C. temperature for 5-7 d. Hypocotyl length may be measured using the ImageJ software (v.1.48v) (https://imagej.nih.gov/ii/).

Within the context of this invention, a plant with an increased root growth is defined as a plant with an increase in average or mean root growth. Preferably, the root growth of said plant is increased by at least 5%, 10%, 15%, 20%, 25%, 30%, 40%, 50%, 60%, 70%, 80%, 90% or higher when compared to the corresponding percentage of a control or reference plant under the same conditions. Root growth may be assessed using techniques known to the skilled person, such as in the experimental part. For root growth analysis, seedlings were grown in vertically placed 0.5MS− media plates in controlled growth conditions (Aralab 600; long days 16:8 h day/night cycle; 22° C.). MyROOT software (Betegón-Putzé et al., 2019) was used to compare root growth of plants. Lateral roots were manually counted under a steriozoom microscope in 9-d-old seedlings. Within the context of this invention, a plant with an increased petiole length is defined as a plant with an increase in average or mean petiole length. Preferably, the increase of petiole length of said plant is increased by at least 5%, 10%, 15%, 20%, 25%, 30%, 40%, 50%, 60%, 70%, 80%, 90% or higher when compared to the corresponding percentage of a control or reference plant under the same conditions. Petiole length may be assessed using techniques known to the skilled person, such as in the experimental part. In short, seedlings may be grown in horizontally placed 0.5MS− media plates in controlled growth conditions (Aralab 600; long days 16:8 h day/night cycle 60% relative humidity and 50-70 μmol m⁻² s⁻¹ of cool-white fluorescent light) at 22° C. or 28° C. temperature for 5-7 d. Hypocotyl length may be measured using the ImageJ software (v.1.48v) (https://imagej.nih.gov/ij/).

Within the context of this invention, a plant with an altered flowering time is defined as a plant with a flowering time which is altered, preferably delayed when compared to the flowering time of a control or reference plant under the same conditions. The delay may be of at least 6 hours, 12, 18, 24 hours or of at least 1 day, 2 days or longer. Flowering time may be assessed using techniques known to the skilled person, such as in the experimental part. In short, two-week-old seedlings grown in 0.5MS− agar plates may be transferred individually to pots containing 30±1 g of substrate (plus 1:8 v/v vermiculite and 1:8 v/v perlite) in normal growth conditions (long days, 22° C.). For flowering time analysis, plants may be photographed and number of plants with >1 cm bolt may be manually counted every day until all plants were bolted and had flowers.

Within the context of this invention, a plant with a modulation of a marker gene involved in phytohormone response is defined as a plant with an increase or a decrease in at least one, two, five, ten, twenty genes involved in phytohormone response. Preferred marker genes in this context are involved in auxin metabolism and transport and include, YUC8, PIN7, PINS, PIN6 (Hentrich M., et al 2013, Lee and Seo 2017, Wang J et al 2021, Sawchuk M G et al 2013, Megan G et al 2013). Preferably, the increase by at least 5%, 10%, 15%, 20%, 25%, 30%, 40%, 50%, 60%, 70%, 80%, 90% or higher when compared to the expression levels in a control or reference plant under the same conditions. Preferably, the decrease by at least 5%, 10%, 15%, 20%, 25%, 30%, 40%, 50%, 60%, 70%, 80%, 90% or higher when compared to the expression levels in a control or reference plant under the same conditions. The increase or the decrease is preferably assessed using techniques known to the skilled person such as PCR or Northern blot or transcriptomic profiling analysis as described in the experimental part.

### Abiotic Stress (b)

In an embodiment, under b) the tolerance of the plant to an abiotic stress (preferably heat stress) has been improved: the plant may have an improved root hydrotropism, an enhanced programmed cell death in root meristems under osmotic stress, a greater hypocotyl growth under heat stress, a greater petiole elongation under heat stress, improved survival rate under heat stress and/or a capacity to accumulate osmoprotectant metabolites in normal conditions and under heat stress.

In an embodiment, under b) the tolerance of the plant to an abiotic stress (preferably heat stress) has been improved: the plant may have an improved root hydrotropism, an enhanced programmed cell death in root meristems under osmotic stress, a greater hypocotyl growth under heat stress, a greater petiole elongation under heat stress, improved survival rate under heat stress and/or a capacity to accumulate metabolites in normal conditions and under heat stress.

In an embodiment, under b) the tolerance of the plant to an abiotic stress (preferably heat stress) has been improved: the plant may have an improved capacity to accumulate metabolites in normal conditions and under heat stress.

Such metabolites may be osmoprotectant and/or relevant for the plant stress response and nutrient efficiency.

In an embodiment, under b) the tolerance of the plant to an abiotic stress (preferably heat stress) has been improved: the plant may have an improved root hydrotropism, an enhanced programmed cell death in root meristems under osmotic stress, a greater hypocotyl growth under heat stress, a greater petiole elongation under heat stress, improved survival rate under heat stress and/or a capacity to accumulate metabolites and/or an improved carbon utilization and/or reduced or repressed photorespiration pathway signaling in normal conditions and under heat stress.

In an embodiment, under b) the tolerance of the plant to an abiotic stress (preferably heat stress) has been improved: the plant exhibits improved carbon utilization and/or reduced or repressed photorespiration pathway signaling in normal conditions and under heat stress.

In an embodiment, under b) the tolerance of the plant to an abiotic stress (preferably heat stress) is a greater hypocotyl growth under heat stress.

A plant of the invention resistant or tolerant to heat stress may be as defined under b).

A plant exhibiting a tolerance to an abiotic stress such as heat or high temperature stress may be called a heat stress tolerant plant or a plant exhibiting an improved heat stress tolerance or an improved survival rate under heat stress conditions.

Heat stress or elevated temperature has already been defined earlier herein. Control plants sensitive for heat stress have been defined earlier herein.

In short, as soon as a plant (or plant species) has a decreased survival rate, fertility and/or yield loss of at least 5%, 10%, 15%, 20%, 30%, 40%, 50%, 60%, 70%, 80%, or at least 90% or more after a period of from 1-15 days, or from 5-15 days during which the temperature is at least 1 to at least 15 or 3 to at least 15 degree Celsius higher than the optimum growth optimum growth temperature, one can say that this plant is sensitive for heat stress. Such a plant may be used as a control or reference plant under these conditions. A plant of the invention has an improved survival rate, fertility and/or yield compared to the survival rate, fertility and/or yield of the control plant as defined in the previous paragraph assessed under the same heat stress conditions. The improvement is of at least 5%, 10%, 15%, 20%, 30%, 40%, 50%, 60%, 70%, 80%, or at least 90% or more measured during the same period of time and under the same stress conditions.

Heat stress tolerance in this specification is meant to encompass a degree of adaptation to heat or elevated temperatures or stress factors associated with elevated temperatures. Examples of stress factors associated with heat or elevated temperatures include increased light intensity, increase of factors associated with greater water evaporation from soil such as decreased water availability or increase in soil solute (salt) concentrations.

Improved heat stress tolerance can be determined by comparing the heat tolerance of a plant according to the invention with the heat tolerance of a wild-type plant or control plant during or after a heat period or during or after a period of elevated temperature. An adaptation can be seen as a change in a gene or protein expression or change in plant physiology in order to cope with a situation of heat stress or elevated temperature. Therefore, a plant may be defined as a heat stress tolerant plant when the expression of at least one gene inked with heat stress is modulated and wherein this modulation of expression is inked with an improved heat stress tolerance as earlier defined herein. Such a gene may be called a heat stress marker gene. Such genes include Heat Stress Factors (HSFs) or Early Response to Desiccation (ERD) genes. HSFs genes may be HSF4 and HSFA4A. ERD genes may be ERD7 and WRKY33 (Perez-Salamo et al., 2014; Pick et al., 2012; Barajas-Lopez et al., 2021; Krishnamurthy et al., 2020).

A plant that survives longer during or after heat stress and a plant with improved survival or growth during or after a heat period in the context of this specification are considered synonymous to each other and used interchangeably.

The person skilled in the art will be aware that survival and improved survival can be determined by manually counting healthy green plants after a period of heat or elevated temperature or counting surviving plants after 7 days of elevated temperature

Within the context of this invention, a plant that survives longer during or after heat stress may be defined as a plant with an increased survival rate compared to the value of survival rate of a control plant. Said increased survival rate is preferably increased by at least 5%, 10%, 15%, 20%, 30%, 40%, 50%, 60%, 70%, 80%, 90% or more compared to the one of the control plant assessed under the same conditions (period of time and heat).

Within the context of the invention, a plant that survives longer during or after heat stress is preferably defined as a plant that is able to survive a longer period of heat than its wild type counterpart.

Improved growth can be seen as increased accumulated biomass, or increased average accumulated biomass. For example the biomass of treated or modified plants as envisioned by the invention compared to the biomass of wild-type plant is higher by 5%, 10%, 15%, 20%, 30%, 40%, 50%, 60%, 70%, 80%, 90% or more. Biomass can be determined be weighing the entire plant or a specified part thereof, such as roots, stem, stalk, leafs, fruit, seeds, or a combination thereof. In this context a period of time is preferably 1, 2, 3, 4, 5, 6, 7, 8, 9, 10, 12, or 13 days longer or 2, 3, 4, 5, 6, 7, 8, 9, 10, 11, 12, 13, 14, 15, 16 weeks or longer.

Within the context of this invention, a plant that survives longer after a period of heat stress is defined as a plant with an increased survival rate after a period of heat stress compared to the survival of a control plant under the same conditions (period of time and conditions). Increased survival, increased survival rate, heat stress, heat stress period, period of time has been earlier defined herein.

Within the context of this invention, a plant wherein a heat stress marker gene has been down modulated, is defined as a plant wherein expression of a heat stress marker gene is modulated by at least 5%, 10%, 15%, 20%, 25%, 30%, 40%, 50%, 60%, 70%, 80%, 90%, 95% or more when compared to the expression of the corresponding marker gene in a control plant under the same conditions (time and heat). A heat stress marker gene for the purpose of the invention is defined as a gene, which is modulated, preferably up regulated under heat conditions in a normal or wild-type plant. Up regulation and down-regulation can be determined by gene arrays, QPCR techniques or Western blot analysis. A gene and/or protein is said to be up regulated when the amount of mRNA transcribed from said gene and/or the amount of protein translated from said mRNA is increased by at least 5%, 10%, 15%, 20%, 25%, 30%, 40%, 50%, 60%, 70%, 80%, 90, or more when compared to the expression of a corresponding marker in a control plant under the same conditions (time and heat). Accordingly, a gene and/or protein is said to be down regulated when the amount of mRNA transcribed from said gene and/or the amount of protein translated from said mRNA is decreased by at least 5%, 10%, 15%, 20%, 25%, 30%, 40%, 50%, 60%, 70%, 80%, 90, or more when compared to the expression of a corresponding marker in a control plant under the same conditions (time and heat). Examples of heat stress marker genes have been defined earlier herein.

Within the context of the invention, under b) the tolerance of the plant to an abiotic stress may also have been improved when a greater hypocotyl growth is observed under abiotic stress such as heat stress. The improvement of hypocotyl growth has already been defined under a). The only difference now is that such a growth is being assessed under abiotic stress such as heat. All these elements have been defined herein.

Within the context of the invention, under b) the tolerance of the plant to an abiotic stress may also have been improved when a greater petiole elongation under abiotic stress such as heat. The improvement of petiole elongation has already been defined under a). The only difference now is that such an improvement is being assessed under abiotic stress such as heat. All these elements have been defined herein.

In an embodiment, under b) the tolerance of the plant to an abiotic stress has been improved also when a capacity to accumulate osmoprotectant metabolites in normal conditions and under heat stress has been observed. Such osmoprotectant metabolites may be amino acids alanine and the osmoprotectant proline (Szabados and Savoure, 2010). Another osmoprotectant is sucrose. The amount of an osmoprotectant (such as alanine and/or proline, or such as alanine and/or proline and/or sucrose) may be increased by at least 5%, 10%, 15%, 20%, 25%, 30%, 40%, 50%, 60%, 70%, 80%, 90%, or more when compared to the amount present in a control plant under the same conditions (time and heat). The presence of alanine or proline may be assessed using techniques known to the skilled person such as those described in the experimental part. For example, gas chromatography associated with mass spectrometry may be used. Similar techniques may be used for assessing the presence of sucrose.

The GC-TOF-MS system comprised of a CTC CombiPAL autosampler, an Agilent 6890N gas chromatograph, and a LECO Pegasus III TOF-MS running in EI+ mode may be used. Metabolites may be identified by comparing to database entries of authentic standardsC:\Users\agupta\Downloads\(2005). Chromatograms may be evaluated using Chroma TOF 1.0 (Leco) Pegasus software may be used for peak identification and correction of RT. The resulting data matrix may be normalized using an internal standard, Ribitol, in 100% methanol (20:1), followed by normalization with the fresh weight of each sample.

In an embodiment, under b) the tolerance of the plant to an abiotic stress may have been improved also when a capacity to accumulate metabolites relevant for the plant stress response and to improve nutrient efficiency in normal conditions and under heat stress has been observed. Examples of such metabolites include sucrose, omithine, urea, arginine, galacturonic acid, glycerol, erythritol, glutamine, maleic acid and beta alanine.

Higher levels of urea can serve as an alternative nitrogen source (Winter et al., 2015). Increase in levels of arginine, a precursor for synthesis of polyamines (e.g. putrescine), or beata-alanine in plants is associated with an improved defence mechanisms against stresses (Winter et al., 2015; Parthasaranthy et al., 2019). Surprisingly, metabolic analysis of the plant of the present invention, which expresses the BRL3 pathway in the phloem (or overexpresses the BRL3 pathway in the phloem or specifically expresses the BRL3 pathway in the phloem), showed that BRL3 signaling can induce metabolic rearrangements (FIG. 44). In ine with this observation, transcriptome analysis of plant deficient in BRL3 expression failed to activate genes involved in redox and abiotic stress responses (FIGS. 29 and 38). Accordingly, the invention relates to a method or a plant wherein expression of the BRL3 pathway in the phloem (or phloem overexpression in the phloem or phloem specific expression of the BRL3 pathway) improves nutrient efficiency in said plant by differentially accumulating the levels of metabolites relevant for the plant stress response and nutrient efficiency, such as sucrose, omithine, arginine, urea, galacturonic acid, glycerol, erythritol, glutamine, beta alanine and/or maleic acid.

The amount of at least one metabolite mentioned herein and relevant for the plant stress response and nutrient efficiency (such as sucrose, omithine, arginine, urea, galacturonic acid, glycerol, erythritol, glutamine, beta alanine and/or maleic acid) may have been increased by at least 5%, 10%, 15%, 20%, 25%, 30%, 40%, 50%, 60%, 70%, 80%, 90%, or more when compared to the amount present in a control plant under the same conditions (time and heat). In an embodiment, the amount of at least two, at least three metabolites may have been increased. In an embodiment, the amount of all metabolites mentioned herein may have been increased. The presence of each of these metabolites may be assessed using techniques known to the skilled person such as those described in the experimental part. For example, gas chromatography associated with mass spectrometry may be used.

In an embodiment, under b) the tolerance of the plant to an abiotic stress (preferably heat stress) has been improved by exhibiting improved carbon utilization and/or a reduced or repressed photorespiration pathway signaling in normal conditions and under heat stress.

As defined herein, “carbon utilization” encompasses the many different ways that carbon oxides, principally carbon dioxide (CO2) can be recycled for further usage in the plant. Further as defined herein, “photorespiration” refers to a respiratory process in which the CO2 fixation machinery of plants, that is the RuBisCO enzyme uses oxygen instead of CO2, wasting part of the energy produced in photosynthesis. Photorespiration avoids the fixation of CO2 by RuBisCO enzyme therefore reducing both the amount of carbon fixed and the energy produced by photosynthesis. Both processes are closely intertwined and are crucial for modulating plant energy status, either in normal or in stress conditions (Kangasjärvi, S. et al, 2012; Timm S. et al, 2021). In this application, data is presented which demonstrates that the photorespiration machinery is repressed in plants of the invention that express, specifically express or that overexpress the BRL3 pathway in the phloem (FIG. 31). Consequently, this surprising observation may suggest that said plant of the invention may display better carbon utilization under stress conditions compared to wildtype plants, thereby reducing or repressing photorespiration pathway (Cui L. L. et al., 2016; Cavanagh A. P. et al., 2021). Accordingly, in an embodiment, the invention relates to a method or a plant wherein expression of the BRL3 pathway in the phloem (or phloem overexpression in the phloem or phloem specific expression of the BRL3 pathway) leads to a plant wherein carbon utilization and/or photorespiration pathway signaling in said plant are affected. In a preferred embodiment of the invention, said plant of the invention exhibits a reduced or repressed photorespiration pathway signaling and an improved carbon utilization.

The improvement of carbon utilization may be of at least 5%, 10%, 15%, 20%, 25%, 30%, 40%, 50%, 60%, 70%, 80%, 90%, or more when compared to the corresponding carbon utilization in a control plant under the same conditions (time and heat).

The reduction or repression of the photorespiration pathway signaling may be of at least 5%, 10%, 15%, 20%, 25%, 30%, 40%, 50%, 60%, 70%, 80%, 90%, or more when compared to the corresponding photorespiration pathway signaling in a control plant under the same conditions (time and heat).

The carbon utilization and/or photorespiration pathway signaling may be assessed using techniques known to the skilled person such as those described in the experimental part. An example of a technique for the assessment of carbon utilization includes the quantification of plant growth under elevated CO2 levels. The improvement of carbon utilization may be translated by at least 5%, 10%, 15%, 20%, 25%, 30%, 40%, 50%, 60%, 70%, 80%, 90%, or more of better plant growth under elevated CO2 levels when compared to the corresponding plant growth in a control plant under the same conditions (time and heat and elevated CO2 levels).

An example of a technique for the assessment of photorespiration pathway signaling includes transcriptomics analysis looking at the expression of genes involved in photorespiration machinery. The reduction or repression of the photorespiration pathway signaling may be synonymous with the reduction or repression of the expression of at least one, two, three, four or more of genes involved in photorespiration machinery. This reduction or repression of the expression level may be of at least 5%, 10%, 15%, 20%, 25%, 30%, 40%, 50%, 60%, 70%, 80%, 90%, or more when compared to the corresponding expression level of the same gene in a control plant under the same conditions (time and heat and elevated CO2 levels).

Within the context of this invention under b), a plant with an improved root hydrotropism is defined as a plant with an increase in average or mean root curvature distribution, or an increase in percentage of roots with a curvature close to 180 degrees. Preferably, a plant with improved root hydrotropism contains a root curvature distribution where the percentage of roots with an angle between 160 and 180 degrees is increased by at least 5%, 10%, 15%, 20%, 25%, 30%, 40%, 50%, 60%, 70%, 80%, 90% or higher when compared to the corresponding percentage of a control or reference plant under the same conditions. Alternatively, a plant with improved root hydrotropism contains a root curvature distribution where the percentage of roots with an angle between 120 and 150 degrees is decreased by 5%, 10%, 15%, 20%, 25%, 30%, 40%, 50%, 60%, 70%, 80%, 90% or higher when compared to the corresponding distribution of a control or reference plant under the same conditions. Root curvature can be measured as described in the experimental part. It may be done by growing seedlings vertically on MS standard medium or 1% MS standard medium and treated during 24-48 h or during 30 min, 1, 2, 3, 4, 5, 6, 7, 8, 9, 10, 11, 12, 13, 14, 15, 16, 17, 18, 19, 20, 21, 22, 23, 24 hours in a gradient concentration of sorbitol, subsequently measuring five-day-old seedling root curvature (or six-day-old or seven-day-old or eight-day-old or nine-day-old or ten-day-old) and analyzing the angle in Image J software as earlier described herein. Period of time and drought have already been defined herein. ½ MS standard medium is MS standard medium, which has been, diluted ½.

In an embodiment, under b) the tolerance of the plant to an abiotic stress may also have been improved when an enhanced programmed cell death in root meristems under osmotic stress has been observed. Such programmed cell death may be enhanced by at least 5%, 10%, 15%, 20%, 25%, 30%, 40%, 50%, 60%, 70%, 80%, 90%, or more when compared to the cell death in root meristems of a control plant under the same conditions (time and heat). Such an effect in the root meristems may be assessed using techniques known to the skilled person such as those used in the examples. A dye may be used that is specific for cell walls of alive cells. An example of such a dye is propidium iodine. When a cell is dead or in process of dying, the dye can enter and stain the whole cell. The amount of cell death may be quantified by assessing the area of the root that have been stained on microscopy pictures (relative to a control, untreated plant). Microscopy may be used.

### Vascular Transport (c)

In an embodiment, within the context of the invention, under c) the vascular transport properties of the plant has been improved when the plant exhibits an increased accumulation of metabolites in the roots and/or a modulation of a marker gene for vascular transporter.

In an embodiment, within the context of the invention, under c) the vascular transport properties of the plant has been improved when the plant exhibits an increased accumulation of metabolites in the roots, a restricted, affected or decreased phloem unloading in the roots and/or a modulation of a marker gene for vascular transporter.

In a preferred embodiment, a plant of the invention exhibits a restricted, affected or decreased phloem unloading in the roots.

A plant will exhibit an increased accumulation of metabolites in the roots when the amount of exogenous sugar in the roots is increased compared to the amount of exogenous sugar present in the roots of control plants (under the conditions). Within the context of this invention, a plant exhibiting an increased accumulation of metabolites in the roots may be a plant, wherein the length of the root is an indication of the status of the accumulation of metabolites, an increase of the length of the root usually correlating with an increased accumulation of metabolites. In an embodiment, the increase is an increase of at least 5%, 10%, 15%, 20%, 25%, 30%, 40%, 50%, 60%, 70%, 80%, 90%, 95% or more when compared to the length of the root in a control plant under the same conditions (time and heat). The presence and therefore the transport of exogenous sugar may be assessed as described in the experimental part. In short, five-days-old uniformly grown seedlings may be moved to a double layer 0.5MS− media set-up in which the top layer media may be supplemented with or without 3% (w/v) sucrose, whereas the bottom media may be devoid of any sugar. The changes in primary root elongation and lateral root count may be measured 3 days after transfer. MyROOT software (Betegón-Putzé et al., 2019) may be used to compare root growth of plants. Lateral roots were manually counted under a steriozoom microscope.

As defined herein, “phloem unloading” may represent a series of bi-directional cell-to-cell transport steps: on one hand transferring phloem-mobile constituents from the phloem to other plant organs/tissues in order to fuel their developmental or resource storage and on the other hand retaining nutrients in shoot tissues (to keep the vascular/phloem solute concentration high, keeping a higher osmotic pressure in shoot tissues while maintaining the water flowing through the plant vascular tissues). This process of maintaining the water flowing through the plant vascular tissues especially under stress conditions is critical as the water availability is low. Phloem unloading is an important physiological process and plays a key role in regulating the growth and development of plants (Ma S. et al. (2019). Therefore, changes in the phloem unloading process may drastically impact nutrient and energy partitioning, and plant growth (Ham and Lucas, 2014). Surprisingly, it is demonstrated in this application that the specific BRL3 pathway expression in the phloem can directly affect phloem unloading (FIGS. 52-53).

Accordingly, the invention relates to a method or a plant wherein expression of the BRL3 pathway in the phloem (or phloem overexpression in the phloem or phloem specific expression of the BRL3 pathway) restricts phloem unloading in the roots. In one embodiment, a plant of the invention wherein phloem unloading is restricted, affected, decreased in the roots.

Accordingly, in a preferred embodiment, a plant of the invention exhibits a restricted, affected or decreased phloem unloading in the roots. Preferably, the decrease is by at least 5%, 10%, 15%, 20%, 25%, 30%, 40%, 50%, 60%, 70%, 80%, 90% or higher when compared to the phloem unloading in a control or reference plant roots under the same conditions. Phloem unloading may be assessed using techniques known to the skilled person. Confocal microscopy of the roots may be carried out; the root having been provided with a fluorescent marker to that is exclusively transported from shoot tissues downward the roots through the phloem. In an embodiment, the technique used in the experimental part is used:

A plant may also exhibit an increased vascular transport when a modulation of a marker gene for vascular transporter is being observed. Within the context of this invention, a plant wherein a vascular transporter gene has been modulated (increased or decreased), wherein the expression of a vascular transporter gene is modulated by at least 5%, 10%, 15%, 20%, 25%, 30%, 40%, 50%, 60%, 70%, 80%, 90%, 95% or more when compared to the expression of the corresponding marker gene in a control plant under the same conditions (time and drought). A vascular transporter marker gene for the purpose of the invention is defined as a gene, which is modulated (up or down regulated) under heat conditions in a control plant. Up regulation and down-regulation can be determined by gene arrays, qPCR techniques or Western blot analysis. A gene and/or protein is said to be up regulated when the amount of mRNA transcribed from said gene and/or the amount of protein translated from said mRNA is increased by at least 5%, 10%, 15%, 20%, 25%, 30%, 40%, 50%, 60%, 70%, 80%, 90, or more when compared to the expression of a corresponding marker in a control plant under the same conditions (time and heat). Accordingly, a gene and/or protein is said to be down regulated when the amount of mRNA transcribed from said gene and/or the amount of protein translated from said mRNA is decreased by at least 5%, 10%, 15%, 20%, 25%, 30%, 40%, 50%, 60%, 70%, 80%, 90, or more when compared to the expression of a corresponding marker in a control plant under the same conditions (time and heat). A preferred vascular transporter marker gene is a gene that encodes a protein that comprises a transporter domain. A transporter domain may be identified using the InterPro database (https://www.ebi.ac.uk/interpro/; Blum et al., 2020). The InterPro unique identifiers may be used to this end (IPR006121, IPR036259, IPR013057, PTHR45824:SF20, IPR017871, IPR005829, IPR000109). Examples of transporter genes in this context are ESL1 (Yamada et al 2010) that provides stress tolerance and ABCB21 (Ienness et al 2019) which can transport auxins.

### Defense Response (d)

Within the context of the invention, under d) the defense response of the plant may have been modulated when the plant exhibits a modulation of a set of defense response genes.

A plant may exhibit an increased defense response when a modulation of a marker gene for defense response is being observed. Within the context of this invention, a plant wherein a defense response gene has been modulated, is defined as a plant wherein expression of a defense response gene is modulated by at least 5%, 10%, 15%, 20%, 25%, 30%, 40%, 50%, 60%, 70%, 80%, 90%, 95% or more when compared to the expression of the corresponding marker gene in a control plant under the same conditions (time and heat). A defense response marker gene for the purpose of the invention is defined as a gene, which is up or down regulated under heat conditions in a control plant. Up regulation and down-regulation can be determined by gene arrays, qPCR techniques or Western blot analysis. A gene and/or protein is said to be up regulated when the amount of mRNA transcribed from said gene and/or the amount of protein translated from said mRNA is increased by at least 5%, 10%, 15%, 20%, 25%, 30%, 40%, 50%, 60%, 70%, 80%, 90, or more when compared to the expression of a corresponding marker in a control plant under the same conditions (time and heat). Accordingly, a gene and/or protein is said to be down regulated when the amount of mRNA transcribed from said gene and/or the amount of protein translated from said mRNA is decreased by at least 5%, 10%, 15%, 20%, 25%, 30%, 40%, 50%, 60%, 70%, 80%, 90, or more when compared to the expression of a corresponding marker in a control plant under the same conditions (time and heat). A preferred defense response marker gene is selected from genes of the JAZ family (preferably JAZ1, 5 and/or 7) (Guo Q., et al (2018)), the FLS2 gene (Chinchilla D et al 2007), the HSFs (preferably HSFB2B, HSFA4A) (Ikeda M et al 2011 and Perez-Salamo et al 2014), YUC9 and IAA29.

In a preferred embodiment, a method has been provided for modulating the growth physiology (feature a)) of the plant and/or for modulating the tolerance of the plant to an abiotic stress (feature b)), the method comprising expressing the BRL3 pathway (preferably specifically expressing or preferably overexpressing) in the phloem of said plant, preferably wherein the gene BRL3 is expressed (preferably specifically expressed or preferably overexpressed) in the phloem.

In a preferred embodiment, a method has been provided for modulating the growth physiology of a plant (feature a)) and/or improving the tolerance of a plant to an abiotic stress (feature b)) by increasing the hypocotyl growth of said plant under heat stress, wherein said method comprises expressing the BRL3 pathway (preferably specifically expressing or preferably overexpressing) in the phloem of said plant. In a preferred embodiment, the gene BRL3 is expressed (preferably specifically expressed or preferably overexpressed) in the phloem.

In another preferred embodiment, a method has been provided for modulating the growth physiology (feature a)) of the plant, the method comprising expressing the BRL3 pathway (preferably specifically expressing or preferably overexpressing) in the phloem of said plant, preferably wherein the gene BRL3 is expressed (preferably specifically expressed or preferably overexpressed) in the phloem.

In a preferred embodiment, a method has been provided for modulating the growth physiology of a plant (feature a)) by increasing the hypocotyl growth of said plant, wherein said method comprises expressing the BRL3 pathway (preferably specifically expressing or preferably overexpressing) in the phloem of said plant. In a preferred embodiment, the gene BRL3 is expressed (preferably specifically expressed or preferably overexpressed) in the phloem.

In another preferred embodiment, a method has been provided for modulating the tolerance of the plant to an abiotic stress (feature b)), the method comprising expressing the BRL3 pathway (preferably specifically expressing or preferably overexpressing) in the phloem of said plant, preferably wherein the gene BRL3 is expressed (preferably specifically expressed or preferably overexpressed) in the phloem.

In a preferred embodiment, a method has been provided for improving the tolerance of a plant to an abiotic stress (feature b)) by increasing the hypocotyl growth of said plant under heat stress, wherein said method comprises expressing the BRL3 pathway (preferably specifically expressing or preferably overexpressing) in the phloem of said plant. In a preferred embodiment, the gene BRL3 is expressed (preferably specifically expressed or preferably overexpressed) in the phloem.

In a preferred embodiment, a method has been provided for improving the tolerance of a plant to an abiotic stress (feature b)) by improving the carbon utilization and/or reducing or repressing the photorespiration pathway signaling of said plant, wherein said method comprises expressing the BRL3 pathway (preferably specifically expressing or preferably overexpressing) in the phloem of said plant. In a preferred embodiment, the gene BRL3 is expressed (preferably specifically expressed or preferably overexpressed) in the phloem.

In a preferred embodiment, a method has been provided for improving the tolerance of a plant to an abiotic stress (feature b)) by improving the capacity of the plan to accumulate metabolites in normal conditions and under heat stress, wherein said method comprises expressing the BRL3 pathway (preferably specifically expressing or preferably overexpressing) in the phloem of said plant. In a preferred embodiment, the gene BRL3 is expressed (preferably specifically expressed or preferably overexpressed) in the phloem.

In a preferred embodiment, a method has been provided for improving the vascular transport properties of the plant (feature c)) by increasing the accumulation of metabolites in the roots, wherein said method comprises expressing the BRL3 pathway (preferably specifically expressing or preferably overexpressing) in the phloem of said plant. In a preferred embodiment, the gene BRL3 is expressed (preferably specifically expressed or preferably overexpressed) in the phloem.

In a preferred embodiment, a method has been provided for improving the vascular transport properties of the plant (feature c)) by decreasing phloem unloading in the roots, wherein said method comprises expressing the BRL3 pathway (preferably specifically expressing or preferably overexpressing) in the phloem of said plant. In a preferred embodiment, the gene BRL3 is expressed (preferably specifically expressed or preferably overexpressed) in the phloem.

Each feature of these preferred methods has been earlier defined herein.

In a preferred embodiment, the method of expression, preferably specific expression or preferably overexpression of the BRL3 pathway according to the invention is achieved at the gene and/or at the protein level and wherein the expression of at least one gene and/or protein of the BRL3 pathway has been expressed, preferably specifically expressed or preferably overexpressed. The expression (preferably specific expression or preferably overexpression) is preferably carried out in the phloem of said plant.

In a preferred embodiment, the method of expression, preferably specific expression or preferably overexpression concerns the BRL3 gene that is expressed, preferably specifically expressed or preferably overexpressed at the gene and/or at the protein level. The expression (preferably specific expression or preferably overexpression) of BRL3 is preferably carried out in the phloem of said plant.

Expression, preferably overexpression results in a change in expression of said gene and/or encoded protein. However, a change of activity of a given gene and/or protein is also encompassed.

Specific expression of a gene results in a change in expression of said gene and/or encoded protein in a specific part, organ, tissue of a plant. In this embodiment, it may be possible to detect the expression of said gene in other part, tissue, organ of said plant. Usually the number of other parts, organs, tissues in which expression is still detectable is limited. Limited may mean one or two or three other part, tissue or organ of same plant. A definition for “specific expression in the phloem of a plant” has already been given earlier herein. In a preferred embodiment, specific expression in the phloem means that the expression of a gene is only detected in the phloem (and therefore the expression of this gene is not detectable in other tissues than the phloem).

The BRL3 pathway is defined as each and every gene involved in the downstream signalling of BRL3. Such genes include BRL3, KIN7, ERD14, BES1, BZR1, BIN2. Therefore in one embodiment, the method of the invention comprises the expression, preferably the overexpression (or preferably the specific expression) of at least one of the following genes: BRL3, KIN7, ERD14, BES1, BZR1 and BIN2, or at least two or at least three or at least four or all five.

In an embodiment, the following gene (or genes) is (or are) expressed (or overexpressed) (or specifically expressed) in the phloem:
BRL3,
BRL3 and KIN7
BRL3 and ERD14
BRL3 and BES1
BRL3 and BZR1
BRL3 and BIN2
KIN7
ERD14
BES1 and BZR1
BIN2
BRL3, KIN7, ERD14, BES1, BZR1 and BIN2.

Therefore in an embodiment, the method is such that one, two, three, four or five genes of the BRL3 pathways is expressed (preferably specifically expressed or preferably overexpressed). In an embodiment, the expression (preferably specific expression or preferably overexpression) is in the phloem of said plant.

Such expression (preferably specific expression or preferably overexpression) may result in up regulation of the BRL3 pathway by a modulation of expression at the gene and/or protein level resulting in: a modulation at the gene and/or protein level leading to an increase in synthesis of a steroid hormone, an increase in BRL3 perception, an increase in signalling associated with BRL3 and/or an increase in gene transcription. The same holds for other genes of the BRL3 pathway whose expression may be upregulated.

In an embodiment of the invention, synthesis of BRL3 is upregulated (or increased) to at least 0.5%, 1%, 1.5%, 2%, 3%, 4%, 5%, 10%, 15%, 20%, 25%, 30%, 40%, 50%, 60%, 70%, 80% or more, preferably over an extended period of time of 1, 2, 3, 4, 5, 6, 7, 8, 9, 10, 11, 12, 13 days or 2, 3, 4, 5, 6, 7, 8, 9, 10, 11, 12, 13, 14, 15, 16 weeks or longer, when compared to the synthesis of BRL3 in a control plant over the same period of time and under the same conditions. The same holds for other genes of the BRL3 pathway whose expression may be upregulated.

An upregulation of synthesis can be determined by measuring the amount of BRL3 available in the phloem, or in an organ or tissue comprising some phloem and comparing that amount to the amount synthesized in a comparable organ or tissue of a related control plant. The phloem may be considered as a tissue, preferably a vascular tissue. Phloem companion cells are cells that belong to the phloem and therefore the BRL3 pathway may be expressed, overexpressed or specifically expressed in such cells. Accordingly, such expression, overexpression or specific expression may be assessed in such cells. Preferred organs are roots, stems, stalks, leaves, petals, fruits, seeds, tubers, pollen, meristems, callus, sepals, bulbs and flowers. The person skilled in the art will be aware that BRL3 can be quantified using radioactivity labeling (Wang et al 2001, Caño-Delgado et al. 2009). The same holds for other genes of the BRL3 pathway whose expression may be upregulated. A marker gene for the phloem includes SUC2.

In an embodiment, BRL3 signalling is upregulated (or increased) to at least 0.5%, 1%, 1.5%, 2%, 3%, 4%, 5%, 10%, 15%, 20%, 25%, 30%, 40%, 50%, 60%, 70%, 80% or more, preferably over an extended period of time of 1, 2, 3, 4, 5, 6, 7, 8, 9, 10, 11, 12, 13 days or 2, 3, 4, 5, 6, 7, 8, 9, 10, 11, 12, 13, 14, 15, 16 weeks or longer, when compared to a control plant over the same period of time. The same holds for other genes of the BRL3 pathway whose expression may be upregulated.

A change in BRL3 signalling can encompass a change in any of the signalling components of such pathway, such BRL3 signalling may refer to a kinase or enzyme activity, phosphorylation state, protein-protein interactions, cellular localization, regulation of transcription, DNA binding or RNA binding. The person skilled in the art is aware of assays to assess signalling, such as measuring a kinase activity, phosphorylation of a key signalling component or gene transcription of a regulated gene. Examples of polypeptides whose signalling may be upregulated include at least one of BRL3, KIN7, ERD14, BES1, BZR1 and BIN2.

In a preferred embodiment, the method comprises expressing, preferably overexpressing BRL3 signalling according to the invention. The expression (preferably specific expression or preferably overexpression) may be in the phloem of said plant or in an organ or tissue comprising some phloem tissue. Said expression, preferably overexpression (or preferably specific expression) is achieved at the gene and/or protein level, and wherein at least one, preferably two, more preferably three, most preferably four genes and/or proteins of the BRL3 pathway has been overexpressed, the genes and/or proteins being selected from the group consisting of: BRL3, KIN7, ERD14, BES1, BZR1 and BIN2.

Overexpression and upregulation (or specific expression) are considered synonymous and could be interchangeably used within the context of the application. Both terms are defined in the general part dedicated to the definition.

The expression of BRL3 or of a gene of the BRL3 pathway may mean that the plant already expresses an endogenous BRL3 or an endogenous gene of the BRL3 pathway or all genes of the BRL3 pathway. The invention therefore contemplates the expression (preferably the specific expression or preferably the overexpression) of BRL3 or of a gene of the BRL3 pathway in this genetic context: there are two BRL3 and possible two BRL3 pathways coexisting (the endogenous and the exogenous). In this embodiment, the expression (preferably specific expression or preferably overexpression) is preferably in the phloem or in an organ or tissue comprising some phloem tissue of said plant. The expression (preferably specific express or preferably overexpression) may be detectable in phloem companion cells.

In another embodiment, the endogenous expression of BRL3 (or an endogenous gene of the BRL3 pathway or all genes of the BRL3 pathway) has been inactivated or reduced. The invention therefore contemplates the expression (preferably specific expression or preferably the overexpression) of BRL3 or of a gene of the BRL3 pathway in this genetic context: there is one BRL3 and one BRL3 pathway. In this embodiment, the expression (preferably specific expression or preferably overexpression) is preferably in the phloem or in an organ or tissue comprising some phloem tissue of said plant.

Expression (preferably specific expression or preferably overexpression) in the phloem may be achieved by any means known to the skilled person. In an embodiment, the nucleic acid sequence encoding BRL3 or encoding a gene of the BRL3 pathway is under the control of a phloem specific promoter. Suitable phloem specific promoters include the promoter of the SUC2, WOL, PIN1, CVP2, APL gene. A preferred phloem promoter is the SUC2 promoter. A SUC2 promoter sequence is represented by SEQ ID NO:13.

Therefore in an embodiment, the expression (preferably specific expression or preferably overexpression) is carried out by the use of a phloem specific promoter, preferably of the SUC2, WOL, PIN1, CVP2, PEAR1, SAPL, CALS7 or CALS8 gene. In another embodiment, the expression (preferably specific expression or preferably overexpression) is carried out by the use of a phloem specific promoter, preferably of the SUC2, WOL, PIN1, CVP2, PEAR1, SAPL, CALS7, CALS8 gene or NAC86 gene. A WOL promoter sequence is represented by SEQ ID NO:14. A PIN1 promoter sequence is represented by SEQ ID NO:17. A SAPL promoter sequence is represented by SEQ ID NO:18. A CVP2 promoter sequence is represented by SEQ ID NO:19. A PEAR1 promoter sequence is represented by SEQ ID NO:20. A CALS7 promoter sequence is represented by SEQ ID NO:21. A CALS8 promoter sequence is represented by SEQ ID NO:22. A NAC86 promoter is represented by SEQ ID NO: 23. Each of the promoter sequences represented by SEQ ID NO:13, 14, 17, 18, 19, 20, 21, 22, 23 originates from Arabidopsis thaliana. As defined herein, “a phloem specific promoter” is a promoter that is active or functional in the phloem or phloem tissue of a plant and therefore allows phloem-specific expression or phloem expression or phloem overexpression of a gene or coding sequence operably inked to it. A phloem specific promoter regulates the expression of a gene (or coding sequence) primarily in the phloem, but can allow detectable level (“leaky”) expression in other plant organs or tissues as well. This leaky expression in other plant tissues may be at a lower detectable expression as compared to the phloem-specific expression, as evaluated on the level of the mRNA or the protein by standard assays known to a person of skill in the art (e.g. Western blot analysis). The maximum number of organs or tissues where leaky expression may be detected is one, two or three. Accordingly, in one embodiment, a phloem specific promoter regulates the expression of the BRL3 pathway in the phloem and in one other plant tissue. In another embodiment, a phloem specific promoter regulates the expression of the BRL3 pathway in the phloem and in two other plant tissues. In another embodiment, a phloem specific promoter regulates the expression of the BRL3 pathway in the phloem and in three other plant tissues. In a preferred embodiment, a phloem specific promoter allows or induces the expression of the BRL3 pathway to the phloem only.

In an embodiment, the method of the invention comprises modulating a plant adaptation trait, the method comprising expressing the BRL3 pathway in the phloem of said plant by a grafting technique (also called a micrografting technique). In an embodiment, the BRL3 gene is expressed (or specifically expressed or overexpressed) in the phloem of said plant.

Each of the other features relating to the method and/or plant of the invention such as those earlier defined herein relating to the expression (or overexpression or specific expression) of the BRL3 pathway in the phloem, each of the traits, features of the plant resulting of the expression of the BRL3 pathway in the phloem and the expression vector and the promoter used also apply for the method and/or plant obtained using a grafting technique as way of expressing the BRL3 pathway in the phloem of a plant

As defined herein, “a grafting technique”, “grafting” or “a grafting procedure” may be considered as transplanting tissues or organs from one plant to another, wherein said tissues or organs are at very early developmental stages (Bartusch K., 2020). Since this grafting technique is applied to very young plants (such as 3, 4, 5, 6, 7 or 8 day-old-seedlings), it may be called micrografting.

Grafting or micrografting may be carried out as described in the experimental part. A person skilled in the art would understand that said (micro)grafting technique allows for the introduction of different traits between genetically distinct plant organs, as described in Reeves G. et al 2021. Plant organs or tissues to be transplanted may comprise tissues of the shoot system (including leaves, buds, stems, flowers, and fruits) or tissues from the root system (including roots, tubers and rhizomes).

In an embodiment, the grafting is applied to the root. In an embodiment, the transplanted tissue is the shoot. In an embodiment, the grafting is carried out at the root-shoot junction.

In an embodiment, the method of expression concerns the grafting of a shoot (the scion) from one plant to a root system (the rootstock) of another plant.

In a preferred embodiment, the method of expression concerns the grafting or micrografting of a shoot scion expressing the BRL3 pathway to a rootstock of a plant.

In another embodiment, the grafting is applied to the shoot. In an embodiment, the transplanted tissue is the root (or rootstock or root system). In an embodiment, the grafting is carried out at the root-shoot junction.

In an embodiment, the method of expression concerns the grafting of a root (or rootstock or root system) from one plant to a shoot of another plant.

In a preferred embodiment, the method of expression concerns the grafting or micrografting of a root (or rootstock or root system) expressing the BRL3 pathway to a shoot of a plant.

Accordingly a plant obtainable by such a method is encompassed by the invention.

In a method and a plant according to the invention, the BRL3 pathway is upregulated at the gene and/or protein level. Such a transgenic plant with an upregulated BRL3 pathway was prepared in the experimental part. Such a plant may be advantageously used in a method as later defined herein. The identity of genes belonging to the BRL3 pathway has been disclosed earlier herein. In a method and accordingly a plant according to the invention, BRL3 is upregulated at the gene and/or protein level. Such a transgenic plant with an upregulated BRL3 was prepared in the experimental part. Such a plant may be advantageously used in a method as later defined herein In another method and accordingly a plant according to the invention, BRL3, KIN7, ERD14, BES1, BZR1 and/or BIN2 is upregulated at the gene and/or protein level. Such a plant may be advantageously used in a method as later defined herein.

In another method and accordingly a plant according to the invention, BRL3, KIN7, ERD14, BES1, BZR1 and BIN2 are upregulated at the gene and/or protein level. Such a plant may be advantageously used in a method as later defined herein.

Accordingly, a BRL3 gene in the context of the invention is a gene or part of a gene represented by a nucleotide sequence with at least 50%, 60%, 70%, 80%, 90%, 95%, 99% or 100% sequence identity with SEQ ID NO:1 preferably over its entire length. A BRL3 protein is represented by an amino acid sequence with at least 50%, 60%, 70%, 80%, 90%, 95%, 99% or 100% sequence identity or similarity with SEQ ID NO:2 preferably over its entire length, said protein being encoded by said gene, and said protein being capable of binding a brassinosteroid and capable of dimerising with BAK1. BRL3 The person skilled in the art will be aware of assays capable of assays for determining BRL3 activity, such as antibodies recognizing BRL3-BAK1 protein-protein interaction or transient interaction assays in protoplasts or Nicotiana benthamiana leaves (Caño-Delgado et al. 2004, Kinoshita et al. 2005, Fãbregas et al. Plant Cell 2013).

Accordingly, a KIN7 gene in the context of the invention is a gene or part of a gene represented by a nucleotide sequence with at least 50%, 60%, 70%, 80%, 90%, 95%, 99% or 100% sequence identity with SEQ ID NO:3 preferably over its entire length. A KIN7 protein is represented by an amino acid sequence with at least 50%, 60%, 70%, 80%, 90%, 95%, 99% or 100% sequence identity or similarity with SEQ ID NO:4 preferably over its entire length, said protein being encoded by said gene, and said protein has been identified as being the LRR (Leucine Rich Repeat) receptor kinase involved in the BRL3 pathway (see FIG. 20, Table 1) that shows the best affinity for BRL3. It has been identified as a BRL3 interactor in our previously identified BRL3 protein complex (Fàbregas et al., 2013) and was also found to be upregulated upon abiotic stress such as drought stress and ABA treatments (Vialaret et al., 2014, Fàbregas et al., 2018, Grison et al., 2019).

The person skilled in the art will be aware of assays capable of assays for determining KIN7 activity, such as antibodies recognizing BRL3-KIN7 protein-protein interaction.

Accordingly, a ERD14 gene in the context of the invention is a gene or part of a gene represented by a nucleotide sequence with at least 50%, 60%, 70%, 80%, 90%, 95%, 99% or 100% sequence identity with SEQ ID NO:5 preferably over its entire length. A ERD14 protein is represented by an amino acid sequence with at least 50%, 60%, 70%, 80%, 90%, 95%, 99% or 100% sequence identity or similarity with SEQ ID NO:6 preferably over its entire length, said protein being encoded by said gene, and said protein has been identified as being involved in the BRL3 pathway (Fabregas et al 2013 and also be referred to FIG. 23). In an embodiment, an assay for determining an ERD14 activity is an assay as the one depicted in the experimental part and illustrated in FIG. 23 wherein hypocotyl elongation is assessed.

Accordingly, a BES1gene in the context of the invention is a gene or part of a gene represented by a nucleotide sequence with at least 50%, 60%, 70%, 80%, 90%, 95%, 99% or 100% sequence identity with SEQ ID NO:7 preferably over its entire length. A BES1 protein is represented by an amino acid sequence with at least 50%, 60%, 70%, 80%, 90%, 95%, 99% or 100% sequence identity or similarity with SEQ ID NO:8 preferably over its entire length, said protein being encoded by said gene, and said protein being encoded by the gene, being a downstream transcription factors of the BRL3 pathway. The BES1 gene product regulates BL-response gene expression (Nolan et al 2020). Their activity may be assessed as described in Nolan et al.

Accordingly, a BZR1 gene in the context of the invention is a gene or part of a gene represented by a nucleotide sequence with at least 50%, 60%, 70%, 80%, 90%, 95%, 99% or 100% sequence identity with SEQ ID NO:9 preferably over its entire length. A BZR1 protein is represented by an amino acid sequence with at least 50%, 60%, 70%, 80%, 90%, 95%, 99% or 100% sequence identity or similarity with SEQ ID NO:10 preferably over its entire length, said protein being encoded by said gene, and said protein being encoded by the gene, being a downstream transcription factors of the BRL3 pathway. The BZR1 gene product regulates BL-response gene expression (Nolan et al 2020).Their activity may be assessed as described in Nolan et al.

Accordingly, a BIN2 gene in the context of the invention is a gene or part of a gene represented by a nucleotide sequence with at least 50%, 60%, 70%, 80%, 90%, 95%, 99% or 100% sequence identity with SEQ ID NO:11 preferably over its entire length. A BIN2 protein is represented by an amino acid sequence with at least 50%, 60%, 70%, 80%, 90%, 95%, 99% or 100% sequence identity or similarity with SEQ ID NO:12 preferably over its entire length, said protein being encoded by said gene, and said protein being a negative regulator of the BRL3 pathway The chemical inhibitor Bikinin may be used to block its activity. A BIN2 gene encodes a member of the glycogen synthase kinase-3 (GSK3)-like kinases. BIN2 protein functions as a negative regulator of brassinosteroid signaling by phosphorylating and inactivating BES1 and BZR1 transcription factors (Li et al., 2001; He et al., 2002; U and Nam, 2002; Yin et al., 2002; Vert and Chory, 2006; Gampala et al., 2007; Ryu et al., 2007). The person skilled in the art will be aware of assays capable of assays for determining BIN2 activity, such as antibodies recognizing the phosphorylation of BES1 and BZR1 or by assessing their activity.

Each of SEQ ID NO:1, 2, 3, 4, 5, 6, 7, 8, 9, 10, 11 and 12 originates from Arabidopsis thaliana. For each of SEQ ID NO:1, 3, 5, 7, 9, 11 the identity of the sequence used may be at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with each of SEQ ID NO: 1, 3, 5, 7, 9 and 11 respectively.

For each of SEQ ID NO:2, 4, 6, 8, 10 and 12 the identity or similarity of the sequence used may be at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with each of SEQ ID NO: 2, 4, 6, 8, 10 and 12 respectively.

Within the context of the invention, when the BRL3 pathway is increased or upregulated via the increase or upregulation of the gene and/or protein and/or activity level as earlier defined herein, this may be reached by overexpressing the gene as earlier defined using techniques known to the skilled person. The upregulation of the expression of a gene has been further defined in detail in the general part entitled “definitions”, which is present at the end of the description. The generation of transgenic plant expressing or overexpressing a gene may be done using the DNA clone reported in (Caño-Delgado et al., 2004). A construct may be used comprising a gene to be expressed or overexpressed in the transgenic plant. This gene may be cloned using a recombination Gateway Multisite Cloning system (Invitrogen, 12536-017). A promoter, preferably a phloem promoter is used as earlier defined. Both constructs are available in http://gateway.psb.ugent.be (Karimi, M., Bleys, A., Vanderhaeghen, R. and Hilson, P. (2007) Building blocks for plant gene assembly. Plant Physiol. 145, 1183-119). A recombination LR reaction is preferably performed using the three sequenced pENTRY® vectors containing promoter, gene and tag sequences in a three-component pDEST® vector, pB7m34GW. The LR clonase enzyme may facilitate this LR reaction (11791-100; Invitrogen™) and transferred to plants by floral dipping (Clough, and Bent, 1998). In the experimental part of the invention, a preferred method is disclosed for obtaining transgenic plants.

Within the context of the invention, when the BRL3 pathway is increased or upregulated via the increase or upregulation of the gene and/or protein and/or activity level as earlier defined herein, this may be reached by expressing, overexpressing or specifically expressing the gene as earlier defined using grafting techniques known to the skilled person.

In one embodiment, a plant of the invention comprises a plant organ expressing, preferably overexpressing or preferably specifically expressing, the BRL3 pathway, wherein said plant organ=has been grafted to another plant organ=of said plant. More preferably, the transplanted organ is shoot and/or the organ at which the transplantation is being carried out is the root system of a plant. In a preferred embodiment, a plant of the invention comprises a shoot scion expressing the BRL3 pathway, wherein said shoot scion has been grafted onto a rootstock of said plant.

In a further aspect, the invention provides a shoot scion of a plant expressing the BRL3 pathway (or expressing the BRL3 pathway in the phloem of said shoot scion, or overexpressing the BRL3 pathway in the phloem of said shoot scion or has a BRL3 pathway which is specifically expressed in the phloem of said shoot scion).

In an embodiment, the invention provides a shoot scion of a plant expressing the BRL3 gene (or expressing the BRL3 gene in the phloem of said shoot scion, or overexpressing the BRL3 gene in the phloem of said shoot scion or has the BRL3 gene which is specifically expressed in the phloem of said shoot scion). Each feature of this aspect has been earlier defined herein.

In a further aspect, the invention provides a root (or rootstock or root system) of a plant expressing the BRL3 pathway (or expressing the BRL3 pathway in the phloem of said root (or rootstock or root system), or overexpressing the BRL3 pathway in the phloem of said root (or rootstock or root system) or has a BRL3 pathway which is specifically expressed in the phloem of said root (or rootstock or root system).

In an embodiment, the invention provides a root (or rootstock or root system) of a plant expressing the BRL3 gene (or expressing the BRL3 gene in the phloem of said root (or rootstock or root system), or overexpressing the BRL3 gene in the phloem of said root (or rootstock or root system) or has the BRL3 gene which is specifically expressed in the phloem of said root (or rootstock or root system). Each feature of this aspect has been earlier defined herein.

Accordingly in a preferred method of the invention and plant hence obtainable by this method:
exhibits an adaptation trait that has been modified such as exhibiting a modulation of its physiology, an improvement of its tolerance to an abiotic stress (preferably heat stress), an improvement of its vascular transport and/or a modulation of its defense response and
has an overexpressed gene of the BRL3 pathway in the phloem (or has a BRL3 pathway which is specifically expressed in the phloem), such as BRL3.

A plant obtainable by the present method may also be called a modified plant or an engineered plant of the invention.

In an embodiment, a plant of the invention:
exhibits a modulation of its physiology (preferably has an increased hypocotyl growth) and
has an overexpressed gene of the BRL3 pathway in the phloem (or has a BRL3 pathway which is specifically expressed in the phloem), such as BRL3.

In another embodiment, a plant of the invention:
exhibits an improvement of its tolerance to an abiotic stress (preferably heat stress) and preferably such improvement is an increased hypocotyl growth, and
has an overexpressed gene of the BRL3 pathway in the phloem, (or has a BRL3 pathway which is specifically expressed in the phloem), such as BRL3.

In another embodiment, a plant of the invention:
exhibits an improvement of its tolerance to an abiotic stress (preferably heat stress) and preferably such improvement is an improvement of the carbon utilization and/or a reduction or repression of the photorespiration pathway signaling of said plant,
has an overexpressed gene of the BRL3 pathway in the phloem, (or has a BRL3 pathway which is specifically expressed in the phloem), such as BRL3.

In another embodiment, a plant of the invention:
exhibits an improvement of its tolerance to an abiotic stress (preferably heat stress) and preferably such improvement is an improvement of the capacity of the plan to accumulate metabolites in normal conditions and under heat stress,
has an overexpressed gene of the BRL3 pathway in the phloem, (or has a BRL3 pathway which is specifically expressed in the phloem), such as BRL3.

In another embodiment, a plant of the invention:
exhibits an improvement of its vascular transport properties and
has an overexpressed gene of the BRL3 pathway in the phloem (or has a BRL3 pathway which is specifically expressed in the phloem), such as BRL3.

In another embodiment, a plant of the invention:
exhibits an improvement of its vascular transport properties and preferably such improvement is an increase of the accumulation of metabolites in the roots,
has an overexpressed gene of the BRL3 pathway in the phloem (or has a BRL3 pathway which is specifically expressed in the phloem), such as BRL3.

In another embodiment, a plant of the invention:
exhibits an improvement of its vascular transport properties and preferably such improvement is a decrease of phloem unloading in the roots,
has an overexpressed gene of the BRL3 pathway in the phloem (or has a BRL3 pathway which is specifically expressed in the phloem), such as BRL3.

In another embodiment, a plant of the invention:
exhibits a modulation of its defense response and
has an overexpressed gene of the BRL3 pathway in the phloem (or has a BRL3 pathway which is specifically expressed in the phloem), such as BRL3.

The amino acid sequence of BRL3 may have at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity or similarity with SEQ ID NO: 2.

The nucleotide sequence of BRL3 used may have at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 1.

In an embodiment, a plant comprises an expression vector comprising a nucleic acid sequence encoding a polypeptide of the BRL3 pathway, such as BRL3, KIN7, ERD14, BES1, BZR1 and/or BIN2, wherein said expression vector is suitable for expression in the phloem. In a preferred embodiment, the expression in the phloem is achieved by the presence of a phloem specific promoter, such as a SUC2, WOL, PIN1, CVP2, PEAR1, CALS7, CALS8 or an SAPL promoter or such as a SUC2, WOL, PIN1, CVP2, PEAR1, CALS7, CALS8, NAC86 or an SAPL promoter. The SUC2 promoter is preferred in this context.

In a preferred embodiment, a plant comprises an expression vector comprising a nucleic acid sequence which has at least 50% identity with SEQ ID NO:1 and which is operably inked to a phloem specific promoter, preferably the SUC2 promoter (SEQ ID NO:13).

In a preferred embodiment, a plant comprises an expression vector comprising a nucleic acid sequence which has at least 50% identity with SEQ ID NO:1, 3, 5, 7, 9 and/or 11 and which is operably inked to a phloem specific promoter, preferably the SUC2 promoter (SEQ ID NO:13).

In a preferred embodiment, a plant comprises an expression vector comprising a nucleic acid sequence which has at least 50% identity with SEQ ID NO:1 and 3, 5, 7, 9 and/or 11 and which is operably linked to a phloem specific promoter, preferably the SUC2 promoter (SEQ ID NO:13).

In a preferred embodiment, a plant comprises an expression vector comprising a nucleic acid sequence which has at least 50% identity with SEQ ID NO:1, 3, 5, 7, 9 and 11 and which is operably inked to a phloem specific promoter, preferably the SUC2 promoter (SEQ ID NO:13).

Here also the nucleotide sequence used may have at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: 1, 3, 5, 7, 9 or 11.

Each feature of said plant has been defined earlier herein or is defined in the general part dedicated to the definitions.

The invention (methods or plant) may be applied to any higher or vascular plant. A preferred plant is a monocotyledonous or a dicotyledonous as later defined herein. A more preferred plant is selected from Arabidopsis, Solanum, Glycine, Zea, Cucumis, Phaseolus, Succharum, Vicia, Oryza, Triticum, Persea Americana, Vits Vinifera and Sorghum. Preferred Solanum species include Solanum lycopersicum and Solanum pimpinellifollium.

The invention (methods or plant) could be applied to a cell, tissue, organ, part, seed, fruit, product, progeny, or propagation material of the plant as defined herein. A preferred part of the plant for the purpose of the invention is the root or the root system or the rootstock. Another preferred part of the plant is a shoot scion.

### General Part Dedicated to Definitions

### Definitions

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and nonphosphodiester internucleoside linkages (e.g., peptide nucleic acid (PNA) or thiodiester linkages). In particular, nucleic acids can include, without imitation, DNA, RNA, cDNA, gDNA, ssDNA, dsDNA, ssRNA, dsRNA, non-coding RNAs, hnRNA, premRNA, matured mRNA or any combination thereof. A nucleic acid molecule is represented by a nucleic acid sequence. The terms “nucleic acid sequence” and “nucleotide sequence” as used herein are interchangeable, and have their usual meaning in the art. The term refers to a DNA or RNA molecule in single or double stranded form. An “isolated nucleic acid sequence” refers to a nucleic acid sequence which is no longer in the natural environment from which it was isolated.

The term “gene” means a DNA sequence comprising a region (transcribed region), which is transcribed into an RNA molecule (e.g. an mRNA) in a cell, operably inked to suitable transcription regulating regions (e.g. a promoter, and/or a translation enhancing element of the present invention). A gene may thus comprise several operably inked sequences, such as a promoter, a 5′ non-translated sequence (also referred to as 5′UTR, which corresponds to the transcribed mRNA sequence upstream of the translation start codon) comprising, for example, sequences involved in translation initiation, a (protein) coding region (cDNA or genomic DNA) and a 3′ non translated sequence (also referred to as 3′ untranslated region, or 3′UTR) comprising e.g. transcription termination sites and polyadenylation site (such as e.g. AAUAAA or variants thereof).

It is further understood that, when referring to “sequences” herein, generally the actual physical molecules with a certain sequence of units or subunits (e.g. nucleic acids or amino acids) are referred to.

The sequence information as provided herein should not be so narrowly construed as to require inclusion of erroneously identified bases. The skilled person is capable of identifying such erroneously identified bases and knows how to correct for such errors. In case of sequence errors, the sequence of the polypeptide obtainable by expression of the gene present in Arabidopsis thaliana containing the nucleic acid sequence coding for the polypeptide should prevail.

### Protein

A “wild type” protein can refer to a protein that is naturally occurring and encoded by a germline genome. A species can have one wild type sequence, or two or more wild type sequences (for example, with one canonical wild type sequence and one or more non-canonical wild type sequences). A wild type protein can be a mature form of a protein that has been processed to remove N-terminal and/or C-terminal residues, for example, to remove a signal peptide.

### Amino Acid Sequence

A protein is represented by an amino acid sequence. An amino acid sequence that is “derived from” a wild type sequence or other amino acid sequence disclosed herein can refer to an amino acid sequence that differs by one or more amino acids compared to the reference amino acid sequence, for example, containing one or more amino acid insertions, deletions, or substitutions as disclosed herein.

Within the context of the application a protein is represented by an amino acid sequence and correspondingly a nucleic acid molecule or a polynucleotide is represented by a nucleic acid sequence. Identity and similarity between sequences. Throughout this application, each time one refers to a specific amino acid sequence SEQ ID NO (take SEQ ID NO: Y as example), one may replace it by: a polypeptide represented by an amino acid sequence comprising a sequence that has at least 50% sequence identity or similarity with amino acid sequence SEQ ID NO: Y. Another preferred level of sequence identity or similarity is 60%. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each amino acid sequence described herein by virtue of its identity or similarity percentage with a given amino acid sequence respectively has in a further preferred embodiment an identity or a similarity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the Ike are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose., such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an embodiment, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an embodiment, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used) “Similarity” between two amino acid sequences is determined by comparing the amino acid sequence and its conserved amino acid substitutes of one polypeptide to the sequence of a second polypeptide. Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, P |
| formation | and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gin or His; Asp to Glu; Cys to Ser or Ala; Gin to Asn; Glu to Asp; Gly to Pro; His to Asn or Gin; lie to Lou or Val; Lou to lie or Val; Lys to Arg; Gin or Glu; Met to Leu or Ile; Phe to Met, Leu or Tyr; Ser to Thr; Thr to Ser; Trp to Tyr; Tyr to Trp or Phe; and, Val to Ile or Leu.

A “chimeric gene” (or recombinant gene) refers to any gene, which is not normally found in nature in a species, in particular a gene in which one or more parts of the nucleic acid sequence are present that are not associated with each other in nature. For example, the promoter is not associated in nature with part or all of the transcribed region or with another regulating region.

The term “chimeric gene” is understood to include expression constructs in which a promoter or transcription regulating sequence is operably inked to one or more sense sequences (e.g. coding sequences) or to an antisense (reverse complement of the sense strand) or inverted repeat sequence (sense and antisense, whereby the RNA transcript forms double stranded RNA upon transcription).

A “nucleic acid construct” or “vector” or “plasmid” is herein understood to mean a man-made (usually circular) nucleic acid molecule resulting from the use of recombinant DNA technology and which is used to deliver exogenous DNA into a host cell. Vectors usually comprise further genetic elements to facilitate their use in molecular cloning, such as e.g. selectable markers, multiple cloning sites and the Ike (see below). A nucleic acid construct may also be part of a recombinant viral vector for expression of a protein in a plant or plant cell (e.g. a vector derived from cauliflower mosaic virus, CaMV, or tobacco mosaic virus, TMV).

A “host cell” or a “recombinant host cell” or “transformed cell” are terms referring to a new individual cell (or organism), arising as a result of the introduction into said cell of at least one nucleic acid molecule, especially comprising a gene encoding a desired protein or a nucleic acid sequence which upon transcription yields an antisense RNA for silencing of a target gene/gene family. The host cell may be a plant cell. The host cell may contain the nucleic acid construct as an extra-chromosomally (episomal) replicating molecule, as a non-replicating molecule or comprises the chimeric gene integrated in the nuclear or organellar DNA of the host cell. The term “organism” as used herein, encompasses all organisms consisting of more than one cell, i.e., multicellular organisms as plant.

“Transformation” and “transformed” refers to the transfer of a nucleic acid sequence, generally a nucleic acid sequence comprising a chimeric gene of interest 30 (GOI), into the nuclear genome of a cell to create a “transgenic” cell or organism comprising a transgene. The introduced nucleic acid sequence is generally, but not always, integrated in the host genome. When the introduced nucleic acid sequence is not integrated in the host genome, one may speak of “transfection”, “transiently transfected”, and “transfected”. For the purposes of the present patent specification, the terms “transformation”, “transiently transfected”, and “transfection” are used interchangeably, and refer to stable or transient presence of a nucleic acid sequence into a cell or organism.

As used herein, the term “operably linked” refers to two or more nucleic acid sequence elements that are physically linked and are in a functional relationship with each other. For instance, a promoter is operably inked to a coding sequence if the promoter is able to initiate or regulate the transcription or expression of a coding sequence, in which case the coding sequence should be understood as being “under the control of” the promoter. Generally, when two nucleic acid sequences are operably inked, they will be in the same orientation and usually also in the same reading frame. They usually will be essentially contiguous, although this may not be required.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one skilled in the art to act directly or indirectly to regulate the amount of transcription from the promoter. The promoter does not include the transcription start site (TSS) but rather ends at nucleotide-1 of the transcription site, and does not include nucleotide sequences that become untranslated regions in the transcribed mRNA such as the 5′UTR. The “5′UTR” is the sequence starting with nucleotide 1 of the mRNA and ending with nucleotide −1 of the start codon. It is possible that a regulating part of the promoter is comprised within the nucleotide sequence becoming a 5′UTR; however, in such case, the 5′UTR is still not part of the promoter as herein defined.

As used throughout, “modulation” is meant to refer to an increase (or up regulation) or a decrease (or down regulation) in the indicated phenomenon (e.g., modulation of a biological activity refers to an increase in a biological activity or a decrease in a biological activity) or in a gene and/or protein expression or activity level. Accordingly, a “modulator” in reference to a receptor, may refer to a compound that facilitates an increase or decrease in activity of the recited receptor.

Within the context of the invention, “Expression” refers to the process by which a gene's coded information is converted into the molecules that support the structures and functions of a cell, such as a protein, transfer RNA, or ribosomal RNA. Expressed genes include those that are transcribed into mRNA and then translated into protein and those that are transcribed into RNA but not translated into protein (for example, transfer and ribosomal RNAs). Expression can also refer to expression at the protein level. Expression at the protein level relates to proteins translated from mRNA.

Within the context of the invention, “Up regulation” or “overexpression” refers to any state in which a gene is caused to be transcribed at an elevated rate as compared to the endogenous transcription rate for that gene. In some examples, up regulation additionally includes an elevated rate of translation of the gene compared to the endogenous translation rate for that gene and/or an elevated rate of secretion of the encoded protein compared to the endogenous secretion rate for that protein.

Methods of testing for up regulation are well known in the art, for example transcribed RNA levels can be assessed using reverse transcriptase polymerase chain reaction (RT-PCR) and protein levels can be assessed using sodium dodecyl sulfate polyacrylamide gel electrophoresis (SDS-PAGE) analysis.

Furthermore, a gene is considered to be up regulated when it exhibits elevated activity compared to its endogenous activity, which may occur, for example, through reduction in concentration or activity of its inhibitor or via expression of mutant version with elevated activity. In preferred embodiments, when the host cell encodes an endogenous gene with a desired biochemical activity, it is useful to up regulate an exogenous gene, which allows for more explicit regulatory control in the bioprocessing and a means to potentially mitigate the effects of central metabolism regulation, which is focused around the native genes explicitly.

Furthermore, up regulation can also refer to expression at the protein level, wherein an up regulated protein refers to any state in which a protein is present at an elevated amount as compared to the endogenous amount for that protein. An elevated amount of protein can result from an increase rate of translation or a decreased rate of degradation or a decreased rate of secretion.

An up regulation of a gene and/or protein expression and/or gene and/or protein activity level may be an up regulation of at least 1%, 5%, 10%, 20%, 30%, 40%, 50%, 60%, 70%, 80%, 90%, 100% or more compared to the corresponding level of a control plant assessed under the same conditions using methods known to the skilled person.

Down regulation: Down regulation refers to any state in which a gene is caused to be transcribed at a reduced rate compared to the endogenous gene transcription rate for that gene. In certain embodiments, gene expression is down regulated via expression of nucleic acids, such as antisense oligonucleotides, double-stranded RNA, small interfering RNA, small hairpin RNA, microRNAs, ribozymes, and the like. In some examples, down regulation additionally includes a reduced level of translation of the gene compared to the endogenous translation rate for that gene. Furthermore, a gene is considered to be down regulated when it exhibits decreased activity compared to its endogenous activity, which may occur, for example, through an increase in concentration or activity of its inhibitor, or via expression of mutant version with reduced activity. Methods of testing for down regulation are well known to those in the art, for example the transcribed RNA levels can be assessed using RT-PCR and proteins levels can be assessed using SDSPAGE analysis.

A down regulation of a gene and/or protein expression and/or gene and/or protein activity level may be a down regulation of at least 1%, 5%, 10%, 20%, 30%, 40%, 50%, 60%, 70%, 80%, 90%, 100% compared to the corresponding level of a control plant assessed under the same conditions using methods known to the skilled person.

Furthermore, down regulation can also refer to expression at the protein level, wherein an down regulated protein refers to any state in which a protein is present at an decreased amount as compared to the endogenous amount for that protein. An decreased amount of protein can result from an decreased rate of translation or an elevated rate of degradation or an elevated rate of secretion.

Modulation of expression according to the invention comprises mutation of a gene, deletion of the gene, knock out of a gene, expression of an exogenous gene construct, expression of an antisense nucleotide targeting a gene, targeting a protein with an antibody.

A mutation according to the invention comprises a deletion, insertion or nucleotide substitution within the coding sequence of a gene, an intron or the promoter region.

“Antisense nucleic acid” as used herein refers to a RNA, DNA or PNA molecule that is complementary to all or part of a target primary transcript or mRNA and that blocks the translation of a target nucleotide sequence. An antisense nucleotide may be a microRNA, a siRNA.

Knock-out: A gene whose level of expression or activity has been reduced to a level not detectable by PCR. In some examples, a gene is knocked-out via deletion or replacement of some or all of its coding sequence. In other examples, a gene is knocked-out via introduction or removal of one or more nucleotides into its open-reading frame, which results in translation of a nonsense or otherwise non-functional protein product.

As used herein, the term “plant” refers to either a whole plant, including in general the class of higher plants amenable to transformation techniques, including both monocotyledonous and dicotyledonous plants or a part of a plant such as e.g. roots, stems, stalks, leaves, petals, fruits, seeds, tubers, pollen, meristems, callus, sepals, bulbs and flowers. The term plant as used herein further refers, without limitations, to plant cells in seeds, suspension cultures, embryos, meristematic regions, callus tissue, leaves, roots, shoots, gametophytic and sporophytic tissue, pollen, protoplasts and microspores. Furthermore, all plant tissues in all organs are included in the definition of the term plant as used herein. Plant tissues include, but is not limited to, differentiated and undifferentiated tissues of a plant, including pollen, pollen tubes, pollen grains, roots, shoots, shoot meristems, coleoptilar nodes, tassels, leaves, cotyledonous petals, ovules, tubers, seeds, kernels. Tissues of plants may be in planta, or in organ, tissue or cell culture. As used herein, monocotyledonous plant refers to a plant whose seeds have only one cotyledon, or organ of the embryo that stores and absorbs food. As used herein, dicotyledonous plant refers to a plant whose seeds have two cotyledons. Plants included in the invention are all plants amenable to transformation. The term “plant” as used herein includes algae and micro-algae.

Endogenous: As used herein with reference to a nucleic acid molecule and a particular cell or microorganism refers to a nucleic acid sequence or peptide that is in the cell and was not introduced into the cell using recombinant engineering techniques. For example, a gene that was present in the cell when the cell was originally isolated from nature is considered to be endogenous. A gene is no longer considered endogenous if the control sequences, such as a promoter or enhancer sequences that activate transcription or translation have been altered through recombinant techniques. Endogenous nucleic acid molecule or gene is synonymous of wild type or reference in the context of the invention when one refers to a control plant or a wild type plant or a reference plant.

Exogenous: As used herein with reference to a nucleic acid molecule and a particular cell or microorganism refers to a nucleic acid sequence or peptide that was not present in the cell when the cell was originally isolated from nature. For example, a nucleic acid that originated in a different microorganism and was engineered into an alternate cell using recombinant DNA techniques or other methods for delivering said nucleic acid is considered to be exogenous. A gene is also considered exogenous if the control sequences, such as a promoter or enhancer sequences that activate transcription or translation have been altered through recombinant techniques.

Exogenous nucleic acid molecule or gene is synonymous of modified in the context of the invention when one refers to a plant that has been modified according to a method of the invention.

The indefinite article “a” or “an” thus usually means “at least one”. In this document and in its claims, the verb “to comprise” and its conjugations is used in its non-limiting sense to mean that items following the word are included, but items not specifically mentioned are not excluded. The word “consist” may be replaced by the expression “essentially consist of” indicating that the main components of the invention are present but that additional trivial elements may be present.

The word “about” or “approximately” when used in association with a numerical value (e.g. about 10) preferably means that the value may be the given value (of 10) more or less 0.1% of the value.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

A single nucleotide polymorphism or SNP occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). SNPs are most frequently diallelic. A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

The term “reporter” (or reporter gene or protein) is mainly used to refer to nucleotide sequence encoding visible marker proteins, such as green fluorescent protein (GFP), eGFP, other fluorescent proteins, luciferase, secreted alkaline phosphatase (SEAP), GUS and the like, as well as nptII markers and the Ike.

Any nucleotide sequences capable of hybridising to the nucleotide sequences of the invention are defined as being part of the invention.

Stringent hybridisation conditions are herein defined as conditions that allow a nucleic acid sequence of at least 25, preferably 50, 75 or 100, and most preferably 150 or more nucleotides, to hybridise at a temperature of about 65° C. or of 65° C. in a solution comprising about 1 M salt or 1 M salt, preferably 6×SSC or any other solution having 10 a comparable ionic strength, and washing at 65° C. in a solution comprising about 0.1M salt, or 0.1 M salt or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the 15 specific hybridisation of sequences having about 90% or more sequence identity or at least 90% sequence identity. Moderate hybridization conditions are herein defined as conditions that allow a nucleic acid sequence of at least 50, preferably 150 or more nucleotides, to hybridise at a temperature of about 45° C. or of 45° C. in a solution comprising about 1 M salt or 1 M salt, preferably 6×SSC or any other solution having a comparable ionic strength, and washing at room temperature in a solution comprising about 1 M salt, or 1 M salt preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically be operably linked to another promoter sequence or, if applicable, another secretory signal sequence and/or terminator sequence than in its natural environment.

When used to indicate the relatedness of two nucleic acid sequences the term “homologous” means that one single-stranded nucleic acid sequence may hybridise to a complementary single-stranded nucleic acid sequence. The degree of hybridisation may depend on a number of factors including the extent of identity between the sequences and the hybridisation conditions such as temperature and salt concentration as discussed later. Preferably the region of identity is greater than 5 bp, more preferably the region of identity is greater than 10 bp.

The term “heterologous” when used with respect to a nucleic acid or polypeptide molecule refers to a nucleic acid or polypeptide from a foreign cell which does not occur naturally as part of the organism, cell, genome or DNA or RNA sequence in which it is present, or which is found in a cell or location or locations in the genome or DNA or RNA sequence that differ from that in which it is found in nature. Heterologous nucleic acids or proteins are not endogenous to the cell into which they are introduced, but have been obtained from another cell or synthetically or recombinantly produced. Generally, though not necessarily, such nucleic acids encode proteins that are not normally produced by the cell in which the DNA is transcribed or expressed, similarly exogenous RNA codes for proteins not normally expressed in the cell in which the exogenous RNA is present. Furthermore, it is known that a heterologous protein or polypeptide can be composed of homologous elements arranged in an order and/or orientation not normally found in the host organism, tissue or cell thereof in which it is transferred, i.e. the nucleotide sequence encoding said protein or polypeptide originates from the same species but is substantially modified from its native form in composition and/or genomic locus by deliberate human intervention. Heterologous nucleic acids and proteins may also be referred to as foreign nucleic acids or proteins. Any nucleic acid or protein that one of skill in the art would recognise as heterologous or foreign to the cell in which it is expressed is herein encompassed by the term heterologous nucleic acid or protein. The term heterologous also applies to non-natural combinations of nucleic acid or amino acid sequences, i.e. combinations where at least two of the combined sequences are foreign with respect to each other.

All patent and literature references cited in the present specification are hereby incorporated by reference in their entirety. The following examples are offered for illustrative purposes only, and are not intended to limit the scope of the present invention in any way.

### FIGURE LEGENDS

FIG. 1: Expression of BRL3 receptor under pSUC2 promoter causes overexpression of BRL3 just in phloem cells.

(A) Confocal images showing BRL3 expression pattern and vascular localization in Arabidopsis pBRL3:BRL3-GFP; Col-O and pSUC2:BRL3-GFP; bd3-2 transgenic lines. (B) Relative BRL3 transcript levels in WT (Col-0), the brl3-2 loss of function mutant and pSUC2:BRL3-GFP; brl3-2 plants

FIG. 2: Expression of BRL3 receptor just in phloem cells enhances hypocotyl growth.

(A) Pictures and (B) measurement of hypocotyl elongation growth of WT (Col-0), brl3-2 mutant and 3 independent lines of pSUC2:BRL3-GFP; brl3-2 transgenic seedlings in control growth conditions (16:8 light:dark cycle, 22° C.). Boxplots depict the distribution of 6-day-old hypocotyl lengths. Data from four independent biological replicates (n 2100). Different letters represent significant differences (p-value<0.05) in an ANOVA plus Tukey's HSD test.

FIG. 3: Expression of BRL3 receptor just in phloem cells enhances root growth.

(A) Pictures and, measurement of (B) primary root elongation growth and (C) the number of emerged lateral roots from 9-d-old seedlings of WT (Col-0), brl3-2 mutant and pSUC2:BRL3-GFP; brl3-2 transgenic line grown in control conditions (16:8 light:dark cycle, 22° C.). Boxplots depict the distribution of root lengths and lateral root counts. Data from three independent biological replicates (n≥30). Different letters represent significant differences (p-value<0.05) in an ANOVA plus Tukey's HSD test.

FIG. 4: Expression of BRL3 receptor just in phloem cells increases petiole size

A) Rosette phenotypes and (B) quantification of petiole length in 21-d-old WT (Col-0), brl3-2 mutant and pSUC2:BRL3-GFP; brl3-2 transgenic plants grown in controlled conditions (16:8 light:dark cycle, 22° C.). Boxplots depict the distribution of petiole lengths of rosette leaves. Different letters indicate a significant difference (p-value<0.05) in a one-way ANOVA test plus Tukey's HSD test. Data from three independent biological replicates (n≥30).

FIG. 5: Expression of BRL3 receptor just in phloem cells alters flowering time:

(A) Representative picture showing flowering phenotypes of the 35-d-old soil grown plants of WT, brl3-2, and BRL3ox lines. (B) Quantitative flowering time analysis (days to bolting) over a 35-days period in WT, brl3-2, and BRL3ox plants grown under long day conditions in the greenhouse. Data from five independent biological replicates (n≥50). Different letters represent significant differences (p-value<0.05) in an ANOVA plus Tukey's HSD test.

FIG. 6: Auxin related genes are deregulated upon expression of BRL3 receptor just in phloem cells.

(A) Auxin metabolism transcripts (as annotated in Gene Ontology category GO:0009850, including biosynthesis and catabolism) being deregulated due to the expression of BRL3 uniquely in phloem cells. (B) Auxin transport transcripts (as annotated in Gene Ontology category GO:0080918) being deregulated due to the expression of BRL3 uniquely in phloem cells.

FIG. 7: Expression of BRL3 receptor just in phloem cells promotes root hydrotropism

A) Hydrotropic root curvature response in 6-d-old roots of 6-day-old roots of WT (Col-0), brl3-2, and pSUC2:BRL3-GFP; bd3-2 lines after 24 h of sorbitol-induced osmotic stress (270 mM). B) Discrete distribution of root hydrotropic curvature angles in. Lightest grey depicts roots curved between 0° and 10°, light grey between 10° and 20°, dark grey between 20° and 30°, and black depicts roots that have a curvature of more than 30°. (B) Continuous distribution of root curvature angles. Different letters indicate a significant difference (p-value<0.05) in a one-way ANOVA test plus Tukey's HSD test. Data from three independent biological replicates (n≥30).

FIG. 8: Expression of BRL3 receptor just in phloem cells enhances programmed cell death upon osmotic stress

Five-day-old roots of WT (Col-0), brl3-2 mutant and pSUC2:BRL3-GFP; brl3-2 lines, stained with propidium iodide (PI, red) after 8 h in control (top) or 270 mM sorbitol (bottom) media. Green channel (GFP) shows the localization of the BRL3 membrane protein receptor in the vascular tissues in primary roots.

FIG. 9: Expression of BRL3 receptor just in phloem cells promotes hypocotyl response to high temperature (in vitro)

(A) Pictures and (B) measurement of high temperature induced hypocotyl elongation growth of WT (Col-0), bd3-2 mutant and pSUC2:BRL3-GFP; bd3-2 transgenic seedlings grown at 22° C. or 28° C. for 6 days under LD conditions. Boxplots depict the distribution of hypocotyl lengths in control (dark grey) or elevated temperature (light grey) conditions.

FIG. 10: BRL3 expression under another vasculature-specific promoter WOL is able to rescue thermomorphogenesis phenotypes

(A) Pictures and (B) measurement of high temperature induced hypocotyl elongation growth of WT (Col-0), brl3-2 mutant and 2 independent transgenic lines of pWOL:BRL3-GFP; brl3-2 seedlings grown at 22° C. or 28° C. for 6 days under LD conditions. Boxplots depict the distribution of hypocotyl lengths in control (dark grey) or elevated temperature (light grey) conditions.

FIG. 11: The quiescent center and stem-cell niche specific promoter WOX5 is not able to rescue thermomorphogenesis phenotypes.

(A) Pictures and (B) measurement of high temperature induced hypocotyl elongation growth of WT (Col-0), brl3-2 mutant and 2 independent transgenic lines of pWDX5:BRL3-GFP; brl3-2 seedlings grown at 22° C. or 28° C. for 6 days under LD conditions. Boxplots depict the distribution of hypocotyl lengths in control (dark grey) or elevated temperature (light grey) conditions.

FIG. 12: Expression of BRL3 receptor just in phloem cells promotes petiole response to high temperature.

(A) Petiole elongation phenotypes and (B) quantification in WT (Col-0), brl3-2 mutant and pSUC2:BRL3-GFP; brl3-2 transgenic plants grown at LD 22° C. condition for 2 weeks and transferred to either 22° C. or 28° C. for 10-d. Boxplots depict the distribution of petiole lengths of rosette leaves from 25-day-old plants growing in control (dark green) or elevated temperature (light green) conditions. Data from three independent biological replicates (n>50).

FIG. 13. Survival of pSUC2:BRL3-GFP, brl3-2 plants exposed to heat stress

Pictures showing heat stress survival phenotypes of WT (Col-0), brl3-2 mutant and pSUC2:BRL3-GFP; brl-2transgenic plants: (A) 21-d-old plants grown in LD 22° C. condition, (B) stressed plants after exposure to heat (LD 37° C.) for 10-d, and (C) recovered plants at LD 22° C. for 5-d.

FIG. 14. BRL3 utilizes downstream BIN2 and BES1/BZR1 master regulators of BR pathway.

Graph showing (A) Enrichment in BES1/BZR1 target genes in deregulated genes in pSUC2:BRL3-GFP; brl3-2 shoots. (B) Pictures and (C) measurement of hypocotyl elongation growth response of 6-d-old seedlings of WT (Col-0), brl3-2 mutant and pSUC2:BRL3-GFP; brl3-2 transgenic seedlings grown at 22° C. or 28° C. for 6 days under LD conditions in media supplemented without or with Bikinin (50 μM). Boxplots depict the distribution of 6-day-old hypocotyl lengths in untreated (dark grey) or treated (light grey) conditions. Data from two independent biological replicates (n>50). Different letters represent significant differences (p-value<0.05) in an ANOVA plus Tukey's HSD test. (D) Increased levels of dephosphorylated BES1 at elevated temperatures is dependent on BRL3. Five-days-old seedlings of WT (Col-0), brl3-2, and BRL3ox seedlings grown in LD at 22° C. were transferred to wither LD at 22° C. or LD at 22° C. 28° C. for 24 h, BES1 was analyzed by Western blot hybridization with an anti-BES1 antibody. HIS3 was used as a internal control.

FIG. 15. List of BES1/BZR1 target genes in deregulated genes in pSUC2:BRL3-GFP; brl3-2 shoots

FIG. 16. Expression of BRL3 receptor just in phloem cells regulates genes involved in thermomorphogenesis.

Graph showing (A) Enrichment of PIF4 and ARF6 target genes in deregulated genes in pSUC2:BRL3-GFP; brl3-2 shoots. (B) Venn diagram showing distribution of different BES1, BZR1, PIF4 and ARF6 target genes in deregulated genes in pSUC2:BRL3-GFP; brl3-2 shoots.

FIG. 17. Abiotic stress genes are deregulated upon expression of BRL3 receptor just in phloem cells.

Deployment of genes within “Response to abiotic stimulus” (GO:0009628) term that are also annotated as responsive to salt, water, heat, cold, light and oxidative stress. Colors in the heatmap represent the log 2 fold change of pSUC2:BRL3-GFP; bd3-2 vs bd3-2 shoots.

FIG. 18: Differences in metabolite accumulation due to phloem BRL3 expression (Differences between pSUC2:BRL3-GFP; brl3-2 and brl3-2).

(A) Metabolites whose overall plant levels are different between pSUC2:BRL3-GFP; brl3-2 and brl3-2 in control conditions. Alanine and the important osmoprotectant amino acid proline are accumulated due to BRL3 expression only in the phloem. Contrary, levels of other aminoacids as lysine, glutamine and glutamic acid or carboxylic acids were reduced in pSUC2:BRL3-GFP; brl3-2 plants. (B) Metabolites whose overall plant levels are different between pSUC2:BRL3-GFP; brl3-2 and brl3-2 under elevated temperatures (28° C.).

FIG. 19. Biotic stress genes are deregulated upon expression of BRL3 receptor just in phloem cells.

Deployment of genes within “Response to external biotic stimulus” (GO:00043207) term that are also annotated as responsive to fungus, bacterium, insect, nematode, symbiont and virus. Colors in the heatmap represent the log 2 fold change of pSUC2:BRL3-GFP; brl3-2 vs brl3-2 shoots.

FIG. 20. Computational modeling of membrane interactors of BRL3. (A) Network representation of computationally-modelled protein-protein interactions of BRL3 with experimentally identified BRL3 interactors from Fàbregas et al., 2013. Node size is the absolute fold change of the transcript upon drought and edges width represent the estimated affinity for such interaction. (B) Overall energy calculation for the BRL3 interaction models. Bars represent the estimated global interaction energies for BRL3 interactors. Point are the calculated energies for each cluster of models per each interaction. The interactor with the best affinity is the membrane LRR-RLK AT3G02880, known as KIN7. The second best affinity for BRL3 is the canonical brassinosteroid co-receptor BAK1.

FIG. 21. KIN7 interacts with BRL3 at plasma membrane and shows a phloem expression pattern.

(A) KIN7 interacts in planta with BRL3 at plasma membrane. BiFC images reveal fluorescence only if two proteins are in such close proximity that they can be considered to interact. This is the case of KIN7 and BRL3, that show fluorescence with YFP fusion in both directions. BRL3 and BRI1 proteins were used as negative controls, as they are both plasma membrane proteins but they do not show any fluorescence. (B) KIN7 promoter fusion with GUS tag reveal an expression pattern predominantly phloematic, in the whole seedlings but specifically in roots, hypocotyl and leaves.

FIG. 22. kin7 mutants also show defective hypocotyl thermomorphogenesis

(A) Pictures and (B) measurement of high temperature induced hypocotyl elongation growth of WT (Col-0), brl3-2 mutant kin7-1 mutant and the crossing of brl3-2 x kin7-1. Seedlings grown at 22° C. or 28° C. for 6 days under LD conditions. Boxplots depict the distribution of hypocotyl lengths in control (dark grey) or elevated temperature (light grey) conditions.

FIG. 23. Mutants for ERD14, another membrane interactor of BRL3, also show defective hypocotyl thermomorphogenesis.

(A) Pictures and (B) measurement of high temperature induced hypocotyl elongation growth of WT (Col-0), brl3-2 mutant erd14-1 and erd14-3 mutants and the crossing of brl3-2 xerd14-3. Seedlings grown at 22° C. or 28° C. for 6 days under LD conditions. Boxplots depict the distribution of hypocotyl lengths in control (dark grey) or elevated temperature (light grey) conditions.

FIG. 24. Mutants of newly identified transcription factors downstream of BRL3, also show defective hypocotyl thermomorphogenesis.

(A) Pictures and (B) measurement of high temperature induced hypocotyl elongation growth of computationally-identified set of transcription factors, predicted to function downstream of BRL3 and with phloem-specificity. Bioinformatics approaches were taken based on transcriptomics experiments from Fàbregas et al., 2018. Seedlings grown at 22° C. or 28° C. for 6 days under LD conditions. Boxplots depict the distribution of hypocotyl lengths in control (dark grey) or elevated temperature (light grey) conditions

FIG. 25: Expression of BRL3 receptor just in phloem cells promotes mobilization of sucrose signals from shoot-to-root.

(A) Graphical representation of the experimental set-up. (B) Pictures and, measurement of (B) primary root elongation growth and (C) number of emerged lateral roots from WT (Col-0), brl3-2 mutant and pSUC2:BRL3-GFP; bd3-2 transgenic seedlings grown on normal growth media for 5-d and transferred to a double layer media set-up with top layer media was supplemented with or without 3% (w/v) sucrose. Boxplots depict the distribution of root lengths and lateral root counts. Data from three independent biological replicates (n≥30). Different letters represent significant differences (p-value<0.05) in an ANOVA plus Tukey's HSD test.

FIG. 26: Expression of BRL3 receptor just in the phloem provokes the accumulation of metabolites in the root.

Metabolites whose overall levels do not change but show different organs accumulation between pSUC2:BRL3-GFP; brl3-2 and brl3-2 plants. All metabolites showing differences are accumulated in the roots of pSUC2:BRL3-GFP; brl3-2, suggesting increased transport capability by BRL3 expression just in the phloem. Importantly among these metabolites the osmoprotectant sugar myoinositol is found.

FIG. 27: Key genes with transporter like function are deregulated upon expression of BRL3 receptor just in phloem cells.

Plant transporter transcripts being deregulated due to the expression of BRL3 uniquely in phloem cells. Transcripts whose protein sequences are annotated at least with one of the following InterPro domains: amino acid transporter, oligopeptide transporter, transporter from major facilitator superfamily, sugar transporter or ABC-transporter were taken as criteria for this analysis.

FIG. 28. BRL3 receptor cascade controls thermomorphogenesis. (A) Pictures and (B) measurement of elevated temperature induced hypocotyl elongation growth of WT (Col-0), the brl3-1, brl3-2, brl3-3 and brl3-4 mutants, and BRL3ox seedlings grown at 22° C. or 28° C. for 6 days under LD conditions. (C) Pictures and (D) quantification of hypocotyl epidermal cell length from 6-d-old WT (Col-0), the brl3-1 and brl3-2 mutants and BRL3ox seedlings grown at 22° C. or 28° C. for 6 days under LD conditions. (E) Petiole elongation phenotypes and (F) quantification in 15-day-old WT (Col-0), the brl3-1 and brl3-2 mutants and BRL3ox plant grown at LD 22° C. condition and transferred to either 22° C. or 28° C. for 7-days. (G) Pictures and (H) measurement of elevated temperature induced hypocotyl elongation growth of WT (Col-0), brl3-2, bes1-d and brl3-2xbes1-d seedlings. Boxplots depict the distribution of hypocotyl length (B, H), or Hypocotyl epidermal cell length (D) of 6-d-old seedlings, or petiole lengths (F) of rosette leaves from 22-day-old plants growing in control (dark green) or elevated temperature (light green) conditions. Red line depicts relative hypocotyl or petiole elongation upon high temperature (ratio 28° C./22° C.±s.e.m.). Different letters indicate a significant difference (p-value<0.05) in a one-way ANOVA test plus Tukey's HSD test. Boxplot represent the median and interquartile range (IQR). Whiskers depict Q1−1.5*IQR and Q3+1.5*IQR and points experimental observations. Data from four independent biological replicates (n>50). (I) Western blot showing BES1 phosphorylation status in WT (Col-0) and brl3-2 mutant seedlings at 22° C. or 28° C. Five-days-old seedlings grown in LD at 22° C. were transferred to wither LD at 22° C. or LD at 28° C. for 24 h, BES1 was analyzed by Western blot hybridization with an anti-BES1 antibody.

FIG. 29. BRL3 is required for plant adaption to climate stresses. (A) GO Enrichment analysis based on genes affected by the interaction brl3˜temperature. Size of the node represents the number of annotated genes in a particular category and the color indicates its adjusted p-value upon enrichment test. (B) Deployment of genes affected by the interaction that are annotated as responding to abiotic stimulus [GO:0009628] and in its children categories (Responses to heat [GO:0009408], cold [GO:0009409], water deprivation [GO:0009414], osmotic stress [GO:0006970] and oxygen levels [GO:0070482]). Red means more high temperature-activation in brl3 mutant than in WT (putatively repressed by BRL3) and blue the opposite (putatively activated by BRL3). Small red boxes next to heatmap indicates that the gene is among the direct BES1 or BZR1 targets. (C) Pictures and (D) graphs showing plant survival after short-term heat-shock treatment (150 min at 42° C.), (E) Hydrotropic root curvature response in 6-day-old roots after 24 h of sorbitol-induced osmotic stress (270 mM). (F) Discrete distribution of root hydrotropic curvature angles in the different genotypes. (G) Continuous distribution of root curvature angles. Different letters indicate a significant difference (p-value<0.05) in a one-way ANOVA test plus Tukey's HSD test. Boxplot represent the median and interquartile range (IQR). Whiskers depict Q1−1.5*IQR and Q3+1.5*IQR and points experimental observations. Data from five independent biological replicates (n>100). (H) Pictures showing cell death in root meristematic tissues after short term exposure to osmotic stress (8-10 h), in WT, brl3 and BRL3ox seedlings. Green channel (GFP) shows the localization of the BRL3 membrane protein receptor in the vascular tissues in primary roots. (I) Quantification of cell death in sorbitol-treated root tips. Boxplots show the relative PI staining (sorbitol/control) for each genotype. Averages from three independent biological replicates (n>20). Different letters represent significant differences (p-value<0.05) in an ANOVA plus Tukey's HSD test. Boxplots represent the median and interquartile range (IQR). Whiskers depict Q1−1.5*IQR and Q3+1.5*IQR and points experimental observation.

FIG. 30. Local BRL3 receptor functions at the phloem companion cells. Confocal images showing BRL3 expression pattern in different cell/tissue types (A) Vasculature (pWOL:BRL3-GFP; brl3-2); (B-D) phloem companion cells (pSUC2:BRL3-GFP; brl3-2); (E) root SCN (pWDX5:BRL3-GFP; brl3-2); (F) Epidermis (pGL2:BRL3-GFP; brl3-2); and (G) meristematic zone (pRPS5a:BRL3-GFP; brl3-2). (H) Pictures and (I) measurement of high temperature induced hypocotyl elongation growth of WT (Col-0), brl3-2 mutant, and different cell/tissue specific complementation transgenic seedlings grown at 22° C. or 28° C. for 6 days under LD conditions. Boxplots depict the distribution of hypocotyl lengths in control (dark green) or elevated temperature (light green) conditions. Boxplot represent the median and interquartile range (IQR). Whiskers depict Q1−1.5*IQR and Q3+1.5*IQR and points experimental observations. Different letters indicate a significant difference (p-value<0.05) in a one-way ANOVA test plus Tukey's HSD test. Data from three independent biological replicates (n>50). (J) Elevated temperature response of the BRL3 promoter activity. pBRL3::GUS-GFP seedlings were grown at 22° C. or 28° C. for 6 days under LD conditions followed by GUS staining. BRL3 promoter activity was induced in hypocotyls but reduced in roots upon elevated temperature.

FIG. 31. Phloem BRL3 signaling modulates carbon metabolism and energy flux to control adaptive growth. (A) GO Enrichment analysis based on genes affected by the interaction pSUC2:BRL3-GFP; brl3-temperature i.e. genes that are either over-responding or under-responding to temperature in the pSUC2:BRL3-GFP; brl3 line with respect the brl3 mutant. Size of the node represents the number of annotated genes in a particular category and the color indicates its adjusted p-value upon enrichment test. (B) Metabolites differentially accumulated in shoots of 6-d-old seedlings of brl3 vs. WT and pSUC2:BRL3-GFP; brl3 vs. brl3 at ambient temperature (22° C.). Relative levels of (C) pyruvic acid and (D-G) intermediates of the Tricarboxylic Acid (TCA) cycle: Fumaric acid, Succinic acid, and configurational isomers Malic and Maleic acid, and (H) osmoprotectant amino acid proline in WT, brl3 mutant and pSUC2:BRL3-GFP; brl3 seedling shoot tissues at ambient (22° C.) vs elevated (28° C.) temperatures. Boxplots depict the distribution of indicated metabolite in control (dark green) or elevated temperature (light green) conditions. Different letters indicate a significant difference (p-value<0.05) in a one-way ANOVA test plus Tukey's HSD test. Data from six independent biological replicates. (I) Pictures showing hypocotyl elongation growth response to elevated CO2 (800 ppm) in 6-d-old seedlings of WT, brl3, BRL3ox and pSUC2:BRL3-GFP; brl3 lines. (J) Stress traits matrix for all physiological assays performed on the roots and shoots of WT, brl3, BRL3ox and pSUC2:BRL3-GFP; brl3 lines. Color bar depicts values for scaled data. In all the conditions tested, phloem specific BRL3 expression was able to rescue stress responsive defects of the M3 mutant.

FIG. 32. WT appearance of brl3 mutant seedlings in normal growth conditions. (A) Diagram of BRL3 showing the T-DNA insertion sites corresponding to different mutant alleles used in this study. (B) Relative BRL3 transcript levels in different brl3 mutant alleles. Transcript levels of BRL3 were normalized to that of UBIQUITIN and are indicated as relative values, with that of the WT (Col-0) control set to 1. Data are presented as means±SD calculated from three biological and technical replicates. Different letters represent significant differences (p-value<0.05) in an ANOVA plus Tukey's HSD test. (C) Pictures showing 7-d-old seedlings of WT (Col-0), grown vertically in LD conditions at 22° C. temperature.

FIG. 33. Complementation of thermomorphogenesis defect of brl3 mutant with native BRL3 expression. (A) Pictures and (B) measurement of high temperature induced hypocotyl elongation growth of WT (Col-0), brl3-2 mutant and pBRL3:BRL3-GFP; brl3 complementation lines grown at 22° C. or 28° C. for 6 days under LD conditions. Boxplots depict the distribution of hypocotyl lengths in control (dark green) or elevated temperature (light green) conditions. Boxplot represent the median and interquartile range (IQR). Whiskers depict Q1−1.5*IQR and Q3+1.5*IQR and points experimental observations. Data from three independent biological replicates (n>50). Different letters represent significant differences (p-value<0.05) in an ANOVA plus Tukey's HSD test.

FIG. 34. Accelerated flowering-time phenotypes of brl3 mutants. (A) Representative picture showing flowering phenotypes of the 25-d-old soil grown plants of WT, brl3-2, and BRL3ox lines. (B) Quantitative flowering time analysis (days to bolting) over a 35-days period in WT, brl3-2, and BRL3ox plants grown in control and elevated temperature conditions under long days in the greenhouse. Data from five independent biological replicates (n>50).

FIG. 35. Thermo-morphogenesis phenotypes of mutants of BRI1-like receptors and co-receptor BAK1. (A) Pictures and (B) measurement of high temperature induced hypocotyl elongation growth of WT (Col-0), and single and higher order mutants as well as overexpressor lines of different components of BR receptor complex (BRI1, BRL1, BRL3, BAK1) grown at 22° C. or 28° C. for 6 days under LD conditions. (C) measurement of hypocotyl elongation growth response of 6-d-old seedlings of WT (Col-0), brl3-2 mutant and BRL3ox line grown at 22° C. or 28° C. for 6 days under LD conditions in media supplemented without or with Brassinazole (BRZ220, 1 μM). Boxplots depict the distribution of hypocotyl lengths in control (dark green) or elevated temperature (light green) conditions. Boxplot represent the median and interquartile range (IQR). Whiskers depict Q1−1.5*IQR and Q3+1.5*IQR and points experimental observations. Data from three independent biological replicates (n>50). Different letters represent significant differences (p-value<0.05) in an ANOVA plus Tukey's HSD test.

FIG. 36. BIN2 acts downstream to BRL3 during thermos-morphogenesis. (A) Pictures and (B) measurement of hypocotyl elongation growth response of 6-d-old seedlings of WT (Col-0) and brl3-2 mutant grown at 22° C. or 28° C. for 6 days under LD conditions in media supplemented without or with Bikinin (50 μM). Boxplots depict the distribution of hypocotyl lengths in control (dark green) or elevated temperature (light green) conditions in absence or presence of Bikinin. Boxplot represent the median and interquartile range (IQR). Whiskers depict Q1−1.5*IQR and Q3+1.5*IQR and points experimental observations. Data from three independent biological replicates (n>50). Different letters represent significant differences (p-value<0.05) in an ANOVA plus Tukey's HSD test.

FIG. 37. GO enrichment analysis of differentially regulated genes in brl3-2 at 22° C. in the RNAseq. Size of the node represents the number of annotated genes in a particular category and the color indicates its adjusted p-value upon enrichment test.

FIG. 38. Transcriptome analysis of brl3 mutants at high temperature. (A) Dotplot showing the comparison between transcript fold changes in WT (28° C. vs. 22° C.) and in brl3 (28° C. vs. 22° C.). Note that genes following the diagonal of the plot are responding equally. Red or blue dots represent genes that have been identified as affected by the interaction genotype-temperature in the lineal model. (B) Enrichment in targets of core complex of cell elongation i.e. BAP module (BES1/BZR1, PIF4, ARF6) and HBI1 in genes affected by the interaction br13-temperature.

FIG. 39. BRL3 promoter expression analysis and complementation data. (A-E) GUS staining of pBRL3::GFP-GUS; Col-0 seedlings and (F, G) Confocal images of pBRL3:BRL3-GFP; Col-0 seedlings showing native BRL3 expression pattern in Arabidopsis roots. Confocal images showing BRL3 expression pattern and vascular localization in (H) cotyledon and (I) root of pSUC2:BRL3-GFP; brl3-2 transgenic seedlings. (J) Petiole elongation phenotypes and (K) quantification in WT (Col-0), brl3-2 mutant and pSUC2:BRL3-GFP; brl3-2 transgenic plants grown at LD 22° C. condition for 15-d and transferred to either 22° C. or 28° C. for 7-d. Boxplots depict the distribution of petiole lengths of rosette leaves from 22-d-old plants growing in control (dark green) or elevated temperature (light green) conditions. Different letters indicate a significant difference (p-value<0.05) in a one-way ANOVA test plus Tukey's HSD test. Boxplot represent the median and interquartile range (IQR). Whiskers depict Q1−1.5*IQR and Q3+1.5*IQR and points experimental observations. Data from three independent biological replicates (n>30).

FIG. 40. GO enrichment analysis of differentially regulated genes upon phloem specific BRL3 expression. GO enrichment analysis of genes up or downregulated in pSUC2:BRL3-GFP; brl3-2 vs. brl3-2 at ambient temperature (22° C.) conditions. Size of the node represents the number of annotated genes in a particular category and the color indicates its adjusted p-value upon enrichment test.

FIG. 41. Micrografting experiments showing BRL3 effect on phloem function. (A) Graphical representation of the symplastic unloading of free-GFP using micrografting between pSUC2:GFP scion and rootstock from either WT Col-0 or brl3 mutant seedlings. (B) Live confocal imaging of 6-day-post-grafting Arabidopsis root tips. Boxes account for the ROI area where GFP was measured, using the same ROI for all images placed right above the QC. In the labels, upper line indicates upper part of the chimeric plant after grafting (cotyledons and upper part of the hypocotyl), lower line indicates root part of the chimeric plant (upper part of the hypocotyl and primary root). (C) Mean GFP intensity in the selected area from B. Notice an increased unloading in root tip in brl3 root. Letters depict significant differences following ANOVA with Tukey Post-Hoc HSD test. data from three biological replicates, 9<n≤15.

FIG. 42. Metabolomics analysis of Col-0, brl3 and pSUC2BRL3 under elevated temperatures Schematic representation of the specific metabolic response of (A) shoots and (B) roots of 6-d-old seedlings of WT (Col-0), brl3 and pSUC2:BRL3-GFP; brl3 lines exposed to elevated temperature (28° C.) versus optimal temperature (28° C.) under LD conditions. Colored cells represent the log fold-changes. Statistically significant changes (28° C. vs 22° C.) are denoted by thick black borders on the boxes. Asterisks denote metabolites which have a statistically significant 28° C./22° C. ratio between genotypes. Note that most of the metabolites respond similarly to temperature among genotypes.

FIG. 43. BRL3 signals from phloem regulate plant adaptation to multiple climate stress. (A) Graphs showing hypocotyl elongation growth response to elevated CO2 in 6-d-old seedlings of WT, brl3 and pSUC2:BRL3-GFP; brl3 lines. (B) Pictures showing plant survival phenotypes after short-term heat-shock treatment (150 min at 42° C.), (C) Quantification of survival rates upon heat-shock treatments. (D) Root hydrotropism response, (E) Distribution of the root angles upon hydrotropism response. (F) Cell death in root meristematic tissues after short term exposure to osmotic stress (8-10 h), in WT, brl3 and pSUC2:BRL3-GFP; brl3 seedlings. (G) Quantification of cell damage due osmotic stress. As PI-stained ratio between treated and untreated roots. In all the conditions tested, phloem specific BRL3 expression was able to rescue stress responsive defects of the bd3 mutant.

FIG. 44. Exclusive metabolomics hallmarks of phloem-specific BRL3 expression (pSUC2:BRL3-GFP; brl3-2 transgenic lines) versus the ubiquitous BRL3 overexpression (BRL3ox transgenic lines)

(A-I) 9 metabolites were identified as differentially accumulated in the pairwise comparison pSUC2:BRL3-GFP; brl3-2 vs. BRL3ox. This comparison reveals exclusive metabolomics features derived from phloem-specific overexpression of BRL3. Importantly, metabolites more accumulated in the phloem-specific BRL3 overexpression include key compounds in energy partitioning (sucrose) and organic nitrogen storage (glutamine, omithine, arginine, urea). (J) Metabolites identified as differentially accumulated specifically in the roots.

FIG. 45. BRL3 phosphorylates BRL3 kinase domain. In vitro phosphorylation assays between BRL3 and KIN7 kinase domains. The image show and autoradiography revealing the incorporation of 32P into KIN7 kinase when incubated with BRL3 (left, bottom bands). BRL3 seems to strongly auto-phosphorylate itself (upper bands). The phosphorylation in KIN7 kinase domain is lost when incubated with a phospho-dead version of BRL3, the BRL3T1025A (right, bottom bands). KIN7 is not able to phosphorylate BRL3 domains given that the BRL3 phosphorylation pattern is lost when KIN7 is incubated with the phospho-dead version of BRL3 kinase (right, upper bands).

FIG. 46. KIN7 show better interaction affinity for BRL3 than the canonical brassinosteroid co-receptor BAK1. The protein-protein interaction between KIN7 and BRL3 is also confirmed in Arabidopsis protoplast using FRET-FLIM techniques. (A) Images of cells plasma membrane representing the fluorescence lifetimes of negative control (only BRL3, left) and the interactions BRL3-BAK1 (middle) and BRL3-KIN7 (right). (B) The quantification of lifetimes of >20 images reveal a better FLIM (affinity) for the interaction of BRL3 with KIN7 than with the canonical co-receptor BAK1.

FIG. 47. ERD14 interacts in planta with BRL3 at plasma membrane. BiFC images reveal fluorescence only if two proteins are in such close proximity that they can be considered to interact. This is the case of ERD14 and BRL3, that show fluorescence with YFP fusion in both directions. BRL3 and BKI1 proteins were used as negative controls, as they are both plasma membrane proteins but they do not show any fluorescence.

FIG. 48. ERD14 promoter fusion with GUS tag reveal an expression pattern predominantly vascular in roots, hypocotyl and specially, in leaves.

FIG. 49 Expression of BRL3 in just phloem cells promotes hypocotyl response to high temperatures and recapitulates growth defects in brl3-2 mutant. (A) Confocal microscopy pictures of Arabidopsis transgenic roots expressing BRL3 just in the phloem driven by PEAR1, CALS7, CALS8 and NAC86 promoters. (B) Pictures of high temperature induced hypocotyl elongation growth of WT (Col-0), brl3-2 mutant, BRL3ox and phloem-specific BRL3 transgenic seedlings grown at 22° C. or 28° C. for 6 days under LD conditions.

FIG. 50. Specific BRL3 expressions in endodermis and epidermis are not able to rescue thermo-morphogenesis phenotypes in brl3-2 mutants. (A) Confocal microscopy pictures of Arabidopsis transgenic roots expressing BRL3 just in the epidermis, driven by EXP7 promoter or in the endodermis, driven by SCR promoter. (B) Pictures of high temperature induced hypocotyl elongation growth of WT (Col-0), brl3-2 mutant, BRL3ox and phloem-specific BRL3 transgenic seedlings grown at 22° C. or 28° C. for 6 days under LD conditions.

FIG. 51. Expression of activated forms of BES1 or BZR1 transcription factors just in phloem cells promotes hypocotyl response to high temperatures. (A) Confocal microscopy pictures of Arabidopsis transgenic roots expressing the constitutively-active forms BES1-D and BZR1-D (Yin et al., 2002; Wang et al., 2002) just in the phloem driven by SUC2 promoter. (B) Pictures of high temperature induced hypocotyl elongation growth of WT (Col-0), brl3-2 mutant, BRL3ox and phloem-specific BES1-D and BZR1-D transgenic seedlings grown at 22° C. or 28° C. for 6 days under LD conditions.

FIG. 52. Specific BRL3 expression in the phloem restricts phloem unloading. (A) Schematic representation of the grafting experiment setting. Transgenic plants expressing free GFP in the phloem (driven by SUC2 promoter) are used as shoot scions in the grafts, so GFP is transported downwards through the phloem. Rootstocks of the grafts are Col-0 (Control) or either brl3-2 mutant or phloem-specific BRL3 overexpression pSUC2:BRL3-GFP; brl3-2. The degree of GFP diffusion in the root tips are monitored to reveal the degree of phloem unloading (transport outward vasculature to other tissues) due to BRL3. (B) Confocal microscopy pictures showing the different phloem unloading capabilities of the rootstocks. White field+GFP channel (top panel) and GFP+PI channels (bottom panel). White squares are the ROIs used for GFP diffusion (phloem unloading) quantification.

FIG. 53. Specific BRL3 expression in the phloem restricts phloem unloading (bis). (A) Detailed microscopy pictures of root tips of grafted rootstocks (or ungrafted control at left) illustrating the differences in phloem unloading due to the lack of BRL3 or its phloem-specific overexpression. White squares are the ROIs used for GFP diffusion (phloem unloading) quantification. (B) Boxplots representing the GFP quantification of ˜12 different confocal pictures. Different letters represent significative differences in an ANOVA plus a Tukey's post-hoc test. Specific BRL3 expression in the phloem restrict the degree of phloem unloading in grafted rootstocks.

FIG. 54. Exclusive transcriptomics hallmarks of phloem-specific BRL3 expression (pSUC2:BRL3-GFP; brl3-2 transgenic lines) versus the ubiquitous BRL3 overexpression (BRL3ox transgenic lines). A total of 284 genes were identified as deregulated in the pairwise comparison pSUC2:BRL3-GFP; bd13-2 vs. BRL3ox. This comparison reveals exclusive transcriptomic features derived from phloem-specific overexpression of BRL3. From these 284 transcripts, 130 were expressed more (upregulated) in phloem-specific BRL3 overexpression than in the constitutive BRL3 overexpression whereas 154 were less expressed (down regulated). (A) The 75 most upregulated genes. (B) The 75 most downregulated genes.

TABLE 1
List of membrane interactors of BRL3 computationally modelled

| TAIR ID | Name | Interaction with BRL3 modelled? |
|---|---|---|
| AT1G06840 | AT1G06840 | LRR-RLK |
| AT1G48480 | RKL1 | LRR-RLK |
| AT1G51805 | SIF3 | LRR-RLK |
| AT1G51850 | SIF2 | LRR-RLK |
| AT1G53430 | NILR2 | LRR-RLK |
| AT1G53440 | AT1G53440 | LRR-RLK |
| AT1G63500 | BSK7 | Intracellular kinase |
| AT2G34680 | AIR9 | Other |
| AT2G37050 | AT2G37050 | LRR-RLK |
| AT3G02880 | KIN7 | LRR-RLK |
| AT3G13380 | BRL3 | LRR-RLK |
| AT3G13530 | MAPKKK7 | Intracellular kinase |
| AT3G14840 | LIK1 | LRR-RLK |
| AT3G17410 | CARK1 | Intracellular kinase |
| AT3G23310 | AT3G23310 | Intracellular kinase |
| AT3G24550 | PERK1 | Other RLKs |
| AT3G28450 | BIR2 | LRR-RLK |
| AT3G46290 | HERK1 | Other RLKs |
| AT3G51550 | FER | Other RLKs |
| AT3G57530 | CPK32 | Intracellular kinase |
| AT4G00710 | BSK3 | Intracellular kinase |
| AT4G04720 | CPK21 | Intracellular kinase |
| AT4G08850 | MIK2 | LRR-RLK |
| AT4G23180 | CRK10 | Other RLKs |
| AT4G23250 | EMB1290 | Other RLKs |
| AT4G23650 | CDPK3 | Intracellular kinase |
| AT4G33430 | BAK1 | LRR-RLK |
| AT4G35230 | BSK1 | Intracellular kinase |
| AT5G16590 | LRR1 | LRR-RLK |
| AT5G45500 | AT5G45500 | LRR-RLK |
| AT5G45510 | AT5G45510 | LRR-RLK |
| AT5G49760 | AT5G49760 | LRR-RLK |

TABLE 2
List of transcription factors identified to act downstream BRL3

| TAIR ID | Name | NASC ID of the mutant used here |
|---|---|---|
| AT2G01430 | ATHB17 | N657998 |
| AT5G62470 | MYB96 | N570557 |
| AT5G05790 | AT5G05790 | N670503 |
| AT3G10910 | DALF1 | N624824 |
| AT3G04060 | NAC46 | N867711 |
| AT3G09290 | TAC1 | N660714 |
| AT2G20350 | AT2G20350 | N562649 |
| AT3G24310 | MYB305 | N668337 |
| AT1G31050 | AT1G31050 | N675016 |
| AT2G22800 | HAT9 | N583476 |
| AT1G74370 | AT1G74370 | N545398 |

### EXAMPLES

### Materials and Methods

### Plasmid Construction and Plant Transformation

The pSUC2:BRL3-GFP, pWOL:BRL3-GFP, pWDX5:BRL3-GFP, pRPS5A:BRL3-GFP, pGL2-BRL3-GFP, pCALS7:BRL3-GFP, pCALS8:BRL3-GFP, pNAC86:BRL3-GFP, pPEAR1:BRL3-GFP, pSUC2:BES1-GFP, pSUC2:BZR1-GFP, pSUC2:BES1-D-GFP, pSUC2:BZR1-D-GFP and pSUC1:GFP constructs was generated using a recombination Gateway Multisite Cloning system (Invitrogen). DNA sequences were amplified from respective BAC clones with a proofreading DNA polymerase (Platinum pfx polymerase, 11708-013; Invitrogen™). The purified BRL3 gene PCR product was placed by directional cloning into the Gateway pDONR221® donor vector (12536-017; Invitrogen™) by a recombination BP reaction mixing both, the amplified PCR product and the pDONR221 vector, in a 3:1 ratio. The BP clonase enzyme facilitates this BP reaction (11789-020; Invitrogen™). Gateway P4P1R vector containing cell specific promoters pSUC2, pWOL, pWOX5, pEXP7, pSCR, pRPS5A and pGL2 were used as described in (Marques Bueno et al., 2016) (SEQ ID NOs: 13, 14, 15, 25, 60, 61). All the entry clones for cell/tissue specific promoters are available at http://www.ens-lyon.fr/DRP/SICE/Swelline.html. For promoters pCALS7, pCALS8, pNAC86 and pPEAR1 the constructs described in Xie et al., 2010; Ross-Elliott et al., 2017; Furuta et al., 2014 and Miyashima et al., 2019 were used. For BES1 and BZR1 genes and their constitutively active forms, BES1-D and BZR1-D, the sequences described in Yin et al., 2002, Wang et al., 2002 were used. A recombination LR reaction was performed using the two sequenced pENTRY® vectors containing specific promoter and the BRL3 coding sequence were subcloned into the modified pDEST® gateway vector R4pGWB604 which contains a GFP tag and NPT gene for BASTA resistance. The LR clonase enzyme facilitates this LR reaction (11791-100; Invitrogen™). All inserts were fully sequenced to verify that no cloning errors occurred. The constructs were transformed into WT (Col-0) and brl3-2 mutant plants using Agrobacterium tumefaciens (GV3101)-mediated DNA transfer plants by floral dipping (Clough, and Bent, 1998). Wild-type Col-0 seedlings were used as a control (N1092: NASC (Nottingham Arabidopsis Stock Center number)). The brl3-2 TDNA lines is available in the ABRC (SALK_006024C, Arabidopsis Biological Resource Centre, Ohio State University) webpage. DNA rapid extraction protocol was used for all the plant genotyping experiments. Primers used for cloning, genotyping and sequencing are listed in Table 3.

### Genotyping

DNA rapid extraction protocol (Aljanabi S. M. et al 1997) was used for all the plant genotyping experiments. In short, standard PCR conditions were used to genotype all the mutant plants reported in this study. Standard PCR conditions: 55° C. during 30 seconds for the annealing of the primers and 72° C. during 1 minute for the polymerase extension step. GoTaq® Green Mastermix DNA polymerase (M712, Promega) was used. Design of WT primers and knock out primers for mapping the presence or absence of the T-DNA insertion was done by using the http-J/signal.salk.edu/cgi10 bin/tdnaexpress webpage. Primers used are described in Table 3.

### Plant Materials and Growth Conditions

Wild-type Col-0 seedlings were used as a control (N1092: NASC (Nottingham Arabidopsis Stock Center number)). Four different T-DNA insertion mutant alleles of BRL3 were used brl3-1 (Caño-Delgado A. et al., 2004), brl3-2 (SALK_006024C), brl3-3 (SALK_079612) and brl3-4 (SALK_111696) that are available in the ABRC (Arabidopsis Biological Resource Centre, Ohio State University) webpage. The brl3-2 (SALK_006024C) loss-of-function allele was used more for all the experiments described in this study. The seeds of brl1-301, bak1-3, brl1, brl1brl3, brlbrl3bak1, bri1brl1brl3bak1, BRI1ox, BRL1ox, BRL3ox and bes1-d were obtained as described (Fabregas N. et al., 2018). Double mutants bri1-301xbrl3-2 and brl3-2xbes1-d were created by making genetic cross between respective mutant alleles. Complementation line was created by making genetic cross between homozygous pBRL3:BRL3-GFP transgenic line (Fabragas N. et al., 2013) and br13 mutant. Primers used for genotyping are listed in Table 3.

A. thaliana seeds were sterilized with 35% NaClO for 5 min and washed five times for 5 min with sterile dH2O. Sterilized seeds were stratified at 4° C. in the dark for 2-3 d. Seeds were placed on half strength Murashige and Skoog agar medium with vitamins and without sucrose (0.5MS−) solidified with 0.8% agar. Plates were transferred to chambers with controlled growth conditions (16 h of light/8 h of dark; 22° C. temperature, 60% relative humidity and 50-70 μmol m⁻² s⁻¹ of cool-white fluorescent light) and kept vertically.

Pharmacological treatments were performed in plates adding hormones/drugs in the media.

Chemicals used include; 1 μM BRZ220 (Gift from T. Nakano, RIKEN, Japan) or 50 μM Bikinin (200980, Sigma-Aldrich) for 6-d in control (long days, 22° C.) or high temperature (long days, 28° C.) conditions. Hypocotyl length were measured using the Image J software (v.1.48v) (https-/imagej.nih.gov/ij/).

### Hypocotyl and Root Growth Measurements:

For hypocotyl elongation analysis, seedlings were grown in horizontally placed 0.5MS− media plates in controlled growth conditions (Aralab 600; long days 16:8h day/night cycle 60% relative humidity and 50-70 μmol m⁻² s⁻¹ of cool-white fluorescent light) at 22° C. or 28° C. temperature for 5-7d. Plates were photographed and hypocotyl length were measured using the ImageJ software (v.1.48v) (https:/imagej.nih.gov/ij/). For root growth analysis, seedlings were grown in vertically placed 0.5MS− media plates in controlled growth conditions (Aralab 600; long days 16:8h day/night cycle; 22° C.). MyROOT software (Betegón-Putzé et al., 2019) was used to compare root growth of plants. Lateral roots were manually counted under a stereozoom microscope in 9-d-old seedlings.

### Osmotic Stress Induced PCD Analysis

Five-day-old roots grown in control conditions or in 8-10 h of sorbitol were stained with propidium iodide (10 μg/ml, PI, Sigma). PI stains the cell wall (control) and DNA in the nuclei upon cell death (sorbitol). Images were acquired with a confocal microscope (FV1000 Olympus). Cell death damage in primary roots was measured in a window of 500 μm from QC in the middle root longitudinal section (Image J software v.1.48 v) (https:/Imagej.nih.gov/ij/). As an arbitrary setting to measure the stained area, a color threshold ranging from 160 to 255 in brightness was selected.

### Root Hydrotropism

For hydrotropism analysis, the lower part of the agar was removed from the 0.5MS− agar plates at 45° angle to avoid gravitropic effect, and replaced with 0.5MS− agar with 270 mM sorbitol to simulate a situation of reduced water availability in this section of media. 5 d old seedlings grown vertically in 0.5MS− agar were then placed on the hydrotropism media set-up in a manner that their root tips remained at a 5 mm distance from the −/+ sorbitol media junction for 24 h. Root curvature angles were measured and analyzed using the Image J software (v.1.48v) (https://imagej.nih.gov/ij/).

### Phenotype Analysis in Adult Plants

Two-week-old seedlings grown in 0.5MS− agar plates were transferred individually to pots containing 30±1 g of substrate (plus 1:8v/v vermiculite and 1:8v/v perlite) in normal growth conditions (long days, 22° C.). For flowering time analysis, plants were photographed and number of plants with >1 cm bolt were manually counted every day until all plants were bolted and had flowers. For petiole elongation response to high temperatures, 2-week-old seedlings grown in MS1/2 agar plates were transferred individually to pots containing 30±1 g of substrate (plus 1:8v/v vermiculite and 1:8v/v perlite). The plants were let to acclimatize for 2 days in normal growth conditions (long days, 22° C.) before moving to either control (long days, 22° C.) or high temperature (long days, 28° C.) conditions. Petiole elongation growth in the longest rosette leaf was measured in 25d old plants using Image J software (v.1.48v) (https://imaget.nih.gov/ij/). For heat stress survival, 3-week-old plants grown in pots containing 30±1 g of substrate (plus 1:8 v/v vermiculite and 1:8v/v perlite) under normal growth conditions (long days, 22° C.) were photographed and then exposed to heat stress (long days, 37° C.) for 10-days under optimal watering regime. Plants were photographed after stress period finished and transferred back to normal growth conditions (long days, 22° C.) for recovery. Surviving plants were manually counted 5-7 after recovery.

### Phenotype Analysis in Seedlings

For root growth analysis, seedlings were grown in vertically placed 0.5MS− media plates (half strength MS media, with vitamins, without sugar solidified with 0.8% agar) in controlled growth conditions (Aralab 600; long days 16:8h day/night cycle; 22° C.). MyROOT software (Betegón-Putzé et al., 2019) was used to compare root growth of plants. For thermomorphogenic hypocotyl elongation analysis, seedlings were grown in horizontally placed 0.5MS− media plates in controlled growth conditions (Aralab 600; long days 16:8h day/night cycle 60% relative humidity and 70 μmol m−2 s−1 of cool-white LEDs) at control (22° C.) or elevated (28° C.) temperature for 6-d. For Heat-shock assay, 7-d-old, homogenously grown seedlings were subjected to a 150 min heat shock at 42° C. Plants were moved back to control conditions for 5 days to check survival and recovery after heat-shock. For root hydrotropism, 5-d-old homogeneously grown seedlings were placed on 0.5MS− media plates in which lower part of the media was replaced with 0.5MS− media supplemented with 270 mM sorbitol to simulate a situation of reduced water availability and at 45° angle to avoid gravitropism effect. Root curvature angles were measured and analyzed 24 h after using the Image J software For cell death upon osmotic stress, 5-d-old seedlings grown on 0.5MS− media were shifted to either control or 270 mM sorbitol containing media for 8h, roots were stained with propidium iodide (10 μg/ml, PI, Sigma). PI stains the cell wall (control) and DNA in the nuclei upon cell death (sorbitol). Images were acquired with a confocal microscope (FV1000 Olympus). Cell death damage in primary roots was measured in a window of 500 μm from QC in the middle root longitudinal section (Image J). As an arbitrary setting to measure the stained area, a color threshold ranging from 160 to 255 in brightness was selected. To study the effect of elevated CO2 conditions on seedling growth, seedlings were grown in horizontally placed 0.5MS− media plates in controlled growth conditions (Aralab 600; long days 16:8h day/night cycle 60% relative humidity and 70 μmol m−2 s−1 of cool-white LEDs) at ambient CO2 (400 ppm) or elevated CO2 (800 ppm) conditions for 6-d. Hypocotyl length was measured using the ImageJ software (v.1.48 v) (https://imagej.nih.gov/ij/).

### Bikinin Sensitivity Assay

Seedlings were grown in horizontally placed 0.5MS− media supplemented with or without 50 μM Bikinin (200980, Sigma-Aldrich) for 6-d in control (long days, 22° C.) or high temperature (long days, 28° C.) conditions. Hypocotyl length were measured using the Image J software (v.1.48v) (https://imagej.nih.gov/ii/).

### Computational Approaches for Identified New BRL3 Signaling Components

For the modelling of protein-protein interactions involving BRL3 receptor and proteins that co-immuno precipitated with them (Fábregas et al., 2013), a fully automatized pipeline we used in a high-throughput manner. The protein sequences were used to perform homologues searches for a protein pair (query) within a complete set of PPI resolved structures extracted form PDB and 3DiD databases. Sequence redundancy was eliminated. Alignments of both sequences (BRL3 and interactor) were performed separately against the databased using BLAST. Hit ere filtered for high homology and structures modelled using Modeller and the database hits as a template. Interaction affinity prediction, the interface analysed of Rosetta was used. Global interaction affinities for a pair was calculated according the Boltzmann distribution.

For the identification of transcription factors acting downstream BRL3, transcripts levels measured in a BRL3 overexpressor plants were used, both in control conditions and exposed to drought (Fabregas et al., 2018). Candidates showing a combination of the following features were selected: (I) very strong deregulation by BRL3, (ii) Differential response to drought conditions in BRL3ox plants respect Col-0 and (iii) predicted preferential expression in phloem and vascular root tissues (Brady et al., 2007).

### Bimolecular Fluorescence Complementation Assay (BIFC)

To check the interaction of KIN7 with BRL3 and the interaction of ERD14 with BRL3, the full-length DNA sequences of these proteins were fused to both halves of the YFP, already incorporated in the Gateway-compatible destination vectors (pGTQL1211-YN and pGTQL1221-YC). These constructs were transformed in Agrobacterium tumefaciens strain GV3101. Liquid cultures of Agrobacterium (OD-0.7) were co-infiltrated in 3-weeks-old Nicotiana benthamiana leaves. Leaf samples of the plants were observed under the confocal microscopy 48h after the infiltration, seeking for green fluorescent signal.

### Förster Resonance Energy Transfer by Fluorescence Lifetime Imaging (FRET-FLIM) Protein-Protein Interaction Analysis

The coding sequences of BRL3, KIN7 and BAK1 used for FRET-FLIM experiments in N. benthamiana were cloned into Gateway vectors containing the P-estradiol inducible promoter and a C-terminal mVenus or mCherry by LR-reaction (Invitrogen). Agrobacterium tumefaciens strain harbouring p19 silencing repressor plasmid was transformed with above-described plasmids. Transient transformation of N. benthamiana epidermal leaf cells and induction of mVenus or mCherry tagged fusion proteins with β-estradiol was carried out as described before (Wiedtkamp-Peters & Stahl, 2017). FLIM measurements were carried out with a confocal laser scanning microscope (inverted LSM780, ZEISS) equipped with an additional time-correlated single photon counting device with a picosecond time resolution (Hydra Harp 400, PicoQuant). Excitation of mVenus was performed with a pulsed (32 MHz) diode laser at 485 nm and 1 μW at the objective (40× water immersion, C-Apochromat, NA 1.2 Zeiss). Emission was detected at the same objective and detected with SPAD detectors (PicoQuant) using a narrow range bandpass filter (534/35, AHF). A series of 40 frames was acquired of which each image was taken at 12.5 μs pixel dwell time and a resolution of 138 nm/pixel in a 256×256 pixel image. For analysis, 40 frames were merged into one image and analysed using the SymPhoTime software analysis tool ‘GROUPED FLIM’ (PicoQuant). Prior to analysis, a ROI with a threshold of 130 counts was set to exclude background signal from chloroplasts. If necessary, chloroplasts were excluded manually from the RO. A histogram of the fluorescence decay was built from all photons of the RO. For donor only samples, a mono-exponential fit model was used to calculate the fluorescence lifetime of all photons of the ROI, while for FRET samples containing mVenus and mCherry, a bi-exponential fit model was utilized. For reconvolution in the fitting process, potassium iodide-quenched erythrosine was used to measure the instrument response function. FLIM images were created by analysing the fluorescence lifetime of photons from each individual pixel of a merged image with the SymPhoTime software (PicoQuant). Individual pixels are color-coded according to their fluorescence lifetime (Wiedtkamp-Peters & Stahl, 2017).

### In Vitro Phosphorylation Assays

For the in vitro phosphorylation assays, the kinase domains of BRL3 and KIN7 were fused to GST and MBP tags and subcloned into bacterial expression vectors (pDEST-565 and pDEST-566 vectors, Addgene Plasmid #11517 and Plasmid #11520). These were transformed in E. coli expression strain BL21 and grown in large scale volumes (500 ml). Protein expression was induced using 300 uM IPTG at 16° C. overnight. Bacterial cultures were spun down and lysated with lysis buffer (0.5 mg/ml lisozyme+1 mM Pefabloc protease inhibitor+10 mM beta-mercaptoethanol), then sonicated two times. Lysates were centrifuged and the supernatants were transfer to new tubes. For protein purification, 250 uL of amylose beads (NEB, E8012S) were added to the lysates and incubated a 4° C. for 1 h. Then the beads were washed four times and finally the beads-bound proteins were eluted with 10 mM Maltose for 10 min at RT. The eluate was dialyzed in 2 L of PBS two times, 2h each dyalisis at 4° C. The concentration of purified proteins was measured by Bradford assay, checked in PAGE and stored at −80° C. in 10% glycerol. For the phosphorylation the assay performed as described previously (Yin et al., 2002). Briefly, the combination of purified kinase domains were incubated in 20 uL of kinase buffer (20 mm Tris pH 7.5, 100 mm NaCl and 12 mm MgCl2) and 10 uCi 32P-ATP at 37° C. for 1 h. Proteins were resolved by SDS-PAGE and phosphorylation was detected by exposing the dried gel to the phosphor film.

### GUS Staining

For the analysis of the expression pattern of KIN7 and ERD14, the promoter of KIN7 (2kb upstream the start codon SEQ ID NO: 16) and the promoter of ERD 14 (2.6kb upstream the start codon SEQ ID NO: 26) was fused to GFP and GUS, using the Gateway-compatible destination vector R4L1pGWB632. For the analysis of the expression pattern of BRL3, the pBRL3:GFP-GUS reporter was used. The staining protocol was as follows: 6-day-old seedlings were submerged in ice-cold acetone 90% (v/v) and incubated for 20 min on ice. Then acetone was rinsed twice with 1×PBS. Seedlings were incubated with GUS solution [100 mM sodium phosphate buffer pH-7.2, 10 mM sodium EDTA, 0.1% Triton X-100, 1 m/ml 1,5-bromo-chloro-3-indolyl-beta-D-glucorince (Xglu; Duchefa, Harrlem, NL), 10 mM potassium ferrocyanide and potassium ferricyanide] and incubated at 37° C. overnight. Seedlings were rinsed with 1×PBS twice and let overnight in ethanol 70% (v/v). Seedlings were then mounted on slides and coverslides with chloral hydrate. Samples were observed at the following day under a stereomicroscope.

### Root-Ward Sucrose Transport Assay

Five-days-old uniformly grown seedlings were moved to a double layer 0.5MS− media set-up in which the top layer media was supplemented with or without 3% (w/v) sucrose, whereas the bottom media was devoid of any sugar. The seedlings were placed carefully on to the media plates in a way that only the shoot tissue (Cotyledons and hypocotyls) were in contact with the top layer and the root were entirely in the bottom layer media. The changes in primary root elongation and lateral root count were measured 3-d after transfer. MyROOT software (Betegón-Putzé et al., 2019) was used to compare root growth of plants. Lateral roots were manually counted under a steriozoom microscope.

### RT-qPCR

RNA was extracted from the 15-d-old plants of different genotypes using the Plant Easy Mini Kit (Qiagen). The cDNA was obtained from RNA samples by using the Transcriptor First Strand cDNA Synthesis Kit (Roche) with oligo dT primers. The real-time qPCR reactions were performed from 10 ng of cDNA using LightCycler 480 SYBR Green I master mix (Roche) in 96-well plates according to the manufacturers recommendations. Ubiquitin (AT5G56150) was used as housekeeping gene for relativizing expression. Primers used are described in Table 3.

### Transcriptomic Profiling Analysis

For RNAseq analysis, shoot tissue were collected from 6-d-old seedlings grown under control (long days, 22° C.) or high temperature (long days, 28° C.) conditions. Two or three biological replicates were collected per genotype per experimental conditions. RNA was extracted with the Plant Easy Mini Kit (Qiagen) and quality checked using the Bioanalyser. Stranded cDNA libraries were prepared with TruSeq Stranded mRNA kit (Illumina). Paired-end sequencing, with 75-bp reads, was performed in an Illumina HiSeq500 sequencer, at a minimum depth of 32 M. Reads were trimmed 9 bp at their 3′ end and quality filtered using Trimmomatic. Remaining reads were mapped against the TAIR10 genome with “HISAT2”. Mapped reads were quantified at the gene level with “HtSeq” using Araport11 annotation file. Multimapping reads were discared. Counts on chloroplast genes were eliminated prior analysis due to an identified artefact. For differential expression, samples were TMM normalized and statistical values calculated with the “EdgeR” package in R. To obtain genes with a different temperature response between genotypes, a lineal model accounting for the interaction temperature-genotype was applied. Values for the interaction term was evaluated in such comparisons. For pairwise comparisons between pSUC2:BRL3-GFP; brl3-2 and brl3-2 genotypes and between pSUC2:BRL3-GFP; brl3-2 and BRL3ox genotypes, results were filtered for adjusted p-value (FDR)<0.05 and FC>|1.5|.

### Western Blot Analysis

Five-day-old seedlings grown on 0.5MS− plates were collected directly or treated with 10 nM BL for 1 h, or kept in high temperature for 24 h. Forty entire seedlings of 5- or 6-day-old Arabidopsis seedlings were collected from each genotype and each treatment conditions, and ground into powder in liquid nitrogen. Protein extraction buffer (50 mM Tris-Cl, pH 6.8, SD, 5% glycerol) supplemented with 5% v/v beta mercaptoethanol and 1× protease inhibitor cocktail (Roche) was added to each samples followed by centrifugation at 18,000×g at 4° C. for 10 min. The resulting supernatants were transferred to a new microfuge tube. Eight μl of 6× Laemmli's buffer was added to the protein samples to be loaded (40 μl) and boiled at 95□C for 3 min. SDS/PAGE was performed to resolve the protein extracts. Loading of equal amounts of proteins was controlled by Ponceau-staining of the membranes. After electrophoresis, proteins were transferred to a PVDF (polyvinylidene difluoride) membrane (Millipore) using Wet transference method (Bio-Rad) and immune-detected with antibodies recognizing BES1 (Yu et al., 2011), Histone (NEB).

### Metabolite Profiling Analyses

Seedlings were germinated in vertically placed 0.5MS− media plates for 6 days at control (22° C.) or high (28° C.) temperatures under long day growth conditions. Shoot and root tissue from six biological replicates were collected separately for both control and high temperature conditions, and for each genotype (WT, brl3-2, and pSUC2:BRL3-GFP; brl3-2). Approximately 500 independent seedlings were bulked in each biological replicate. Frozen plant material was grinded in the Tissue Lyser Mixer-Mill (Qiagen) and were aliquoted into 50 mg samples for both shoot and root tissue samples (the exact weight was annotated for data normalization). Primary metabolite extraction was carried as follows (Lisec et al., 2006). One zirconia and 500 μl of 100% methanol premixed with ribitol (20:1) were added and samples were subsequently homogenized in the Tissue Lyser (Qiagen) 3 min at 25 Hz. Samples were centrifuged 10 min at 14,000 rpm (10° C.) and resulting supernatant was transferred into fresh tubes. Addition of 200 μl of CHCl₃ and vortex ensuring one single phase followed by the addition of 600 μl of H₂O and vortex 15 s. Samples were centrifuged 10 min at 14,000 rpm (10° C.). 100 μl from the upper phase (polar phase) were transferred into fresh eppendorf tubes (1.5 m) and dried in the speed vacuum for at least 3 h without heating. 40 μl of derivatization agent (methoxyaminhydrochloride in pyridine) were added to each sample (20 mg/ml). Samples were shaken for 3 h at 900 rpm at 37° C. Drops on the cover were shortly spun down. One sample vial with 1 ml MSTFA+20 μl FAME mix was prepared. Addition of 70 μl MSTFA+FAMEs in each sample was done followed by shaking 30 min at 37° C. Drops on the cover were shortly spun down. Samples were transferred into glass vials specific for injection in GC-TOF-MS. The GC-TOF-MS system comprised of a CTC CombiPAL autosampler, an Agilent 6890N gas chromatograph, and a LECO Pegasus III TOF-MS running in EI+ mode. Metabolites were identified by comparing to database entries of authentic standards (Kopka et al., 2005). Chromatograms were evaluated using Chroma TOF 1.0 (Leco) Pegasus software was used for peak identification and correction of RT. Mass spectra were evaluated using the TagFinder 4.0 software (Luedemann et al., 2008) for metabolite annotation and quantification (peak area measurements). The resulting data matrix was normalized using an internal standard, Ribitol, in 100% methanol (20:1), followed by normalization with the fresh weight of each sample. Overall metabolite levels were calculated summing up MS signals (non-normalized by fresh weight) in root and shoots, and normalizing by the sum of sample weights of shoot and root of the same replicate (overall sample weight). Pairwise comparisons between pSUC2:BRL3-GFP; brl3-2 and brl3-2 genotypes and between pSUC2:BRL3-GFP; bd3-2 and BRL3ox genotypes were performed through a two-tailed t-test, p-value<0.05 (no multiple testing correction). For inferring metabolite transport between root and shoots, metabolite normalized measure in shoots was divided by overall metabolite level, yielding a matrix within the range [0-1], in which a value of 1 would mean all metabolite is accumulated in the shoot and 0 that all metabolite is accumulated in the roots. A value of 0.5 means equal distribution between shoot and roots. From metabolites whose overall levels are not changing (t-test, p-value>0.05), pairwise comparisons between pSUC2:BRL3-GFP; brl3-2 and brl3-2 shoot/root ratio were performed to reveal differences in metabolite transport and/or accumulation (t-test, p-value<0.05). All data transformation and calculations were performed in R.

### Grafting Experiments and Phloem Unloading Analysis

For grafting experiments, 6 day-old-seedlings were grown under short day conditions (8:16 light:dark cycle) at 22° C. Then, square plates for the grafting were prepared following this order: Two 10 cm*10 cm squares of Whatman® filter paper were soaked with liquid half strength MS media supplemented with 10 g/L sucrose, and then a strip of Hybond blotting paper was placed on it. Excess of liquid MS was poured off the plate. Then, seedlings were placed over the Hybond membrane and grafted with the help of a pair of tweezers and a microblade, as described in Melnyk 2016. Then, the plates were sealed with Parafilm and kept in vertical to recover at 24° in LD conditions. Six days after grafting, seedlings were observed by confocal microscopy using a FV1000 Olympus microscope and a 20× objective. The root was stained with Propidium Iodine (SigmaAldrich) and excited with a 488 nm (GFP) and a 559 nm (PI) laser beams. Pictures for measuring unloading or GFP intensity were taken in the medial plane of the root tip of healthy Arabidopsis seedlings. For measuring the degree of GFP diffusion in the root tips, ImageJ software was used. A ROI (Region of Interest) of 30*50 micrometers was placed at the start of the vascular stem cells of each image, right above the QC, and then the GFP mean intensity of the area was measured in the GFP channel of the .oib file obtained from the confocal microscope. Then, GFP intensity was normalized to Col-0 WT.

TABLE 3
List of Primers used in the application (SEQ ID NO: 53-59, 62-78)

| | | Number of | |
|---|---|---|---|
| Primer name | Sequence (5′ → 3′) | nucleotides | Purpose |
| LBb1.3 | ATTTTGCCGATTTCGGAAC | 19 | Genotyping |
| SEQ ID NO: 53 | | | SALK lines |
| | | | (Universal T- |
| | | | DNA left |
| | | | border |
| | | | primer) |
| SALK_006024_LP | CCAGTGAACTCGTTTGAGCTC | 21 | Forward |
| SEQ ID NO: 54 | | | primer for |
| | | | genotyping |
| | | | brl3-2 mutant |
| | | | allele |
| SALK_006024_RP | TTTATCGAACACTTTGTGGGC | 21 | Reverse |
| SEQ ID NO: 55 | | | primer for |
| | | | genotyping |
| | | | brl3-2 mutant |
| | | | allele |
| RT-BRL3_fwd_2 | ATGAGTGCGGGGATTGTGTT | 20 | Forward |
| SEQ ID NO: 56 | | | primer for |
| | | | checking |
| | | | BRL3 |
| | | | transcript |
| | | | level via RT- |
| | | | qPCR |
| RT-BRL3_rev_2 | CCAGATGTCGGAAGGCTCTC | 20 | Reverse |
| SEQ ID NO: 57 | | | primer for |
| | | | checking |
| | | | BRL3 |
| | | | transcript |
| | | | level via RT- |
| | | | qPCR |
| RT-UBQ30 F | GGTCCGGAAGGCAACCTTT | 19 | Forward |
| SEQ ID NO: 58 | | | primer for |
| | | | UBQ30 |
| | | | housekeeping |
| | | | gene as a |
| | | | endogenous |
| | | | control for |
| | | | RT-qPCR |
| RT-UBQ30 R | CATGGGTCCAGCAGATAGCC | 20 | Reverse |
| SEQ ID NO: 59 | | | primer for |
| | | | UBQ30 |
| | | | housekeeping |
| | | | gene as a |
| | | | endogenous |
| | | | control for |
| | | | RT-qPCR |
| bri4.2 | TTTAGGGTGAGCATGAGATCTCGTGGGCC | 30 | Genotyping |
| SEQ ID NO: 62 | G | | brl3-1 mutation |
| bri4.3 | GAAATCCCTGTAGGAATCGGAAAGCTTGAG | 30 | Genotyping |
| SEQ ID NO: 63 | | | brl3-1 mutation |
| JMRB | GCTCATGATCAGATTGTCGTTTCCCGCCTT | 30 | Genotyping |
| SEQ ID NO: 64 | | | brl3-1 mutation |
| SALK_005982_LP | ATATGGATGTTGCCGAATCTG | 21 | Genotyping |
| SEQ ID NO: 65 | | | brl1-2 mutation |
| SALK_005982_RP | CTGTAAAGCGCCATGACTAGC | 21 | Genotyping |
| SEQ ID NO: 66 | | | brl1-2 mutation |
| SALK_079612_LP | TCCCTAATTGCCAATCTTGAG | 21 | Genotyping |
| SEQ ID NO: 67 | | | brl3-3 mutation |
| SALK_079612_RP | CTGCACGAAAAGACCAAGAAG | 21 | Genotyping |
| SEQ ID NO: 68 | | | brl3-3 mutation |
| SALK_111696_LP | GCTTTACGCGAGTGTCTTGTC | 21 | Genotyping |
| SEQ ID NO: 69 | | | brl3-4 mutation |
| SALK_111696_RP | AGACAACAACCTTGTGGGATG | 21 | Genotyping |
| SEQ ID NO: 70 | | | brl3-4 mutation |
| bri1-301 F | GGAAACCATTGGGAAGATCA | 20 | Genotyping |
| SEQ ID NO: 71 | | | bri1-301 |
| | | | mutation |
| bri1-301 R | GCTGTTTCACCCATCCAA | 18 | Genotyping |
| SEQ ID NO: 72 | | | bri1-301 |
| | | | mutation |
| bes1-D F | TCGACGTCAGCTGCAGCT | 18 | Genotyping |
| SEQ ID NO: 73 | | | bes1-d mutation |
| bes1-D R | ATGGCTTAACTTGGCTGTTCT | 21 | Genotyping |
| SEQ ID NO: 74 | | | bes1-d mutation |
| M13 F | GTAAAACGACGGCCAG | 16 | Sequencing to |
| SEQ ID NO: 75 | | | check M13F |
| | | | region in a |
| | | | plasmid |
| AttR2/AttB2_Fw | GCTTTCTTGTACAAAGTGGT | 20 | Sequencing of |
| SEQ ID NO: 76 | | | the reporter in |
| | | | a plasmid |
| AttR2/AttB2_Rv | ACCACTTTGTACAAGAAAGC | 20 | Sequencing of |
| SEQ ID NO: 77 | | | the CDS insert |
| | | | in a plasmid |
| AtBRL3_gen_Rev | TCCGGAGGAGAAGTTGTTTC | 20 | Genotyping of |
| SEQ ID NO: 78 | | | transgenic |
| | | | plants |

### Results

### BRL3 Receptor Driven by the SUC2 Promoter Results in Plants Expressing Higher Levels of BRL3 Specifically in the Phloem Companion Cells

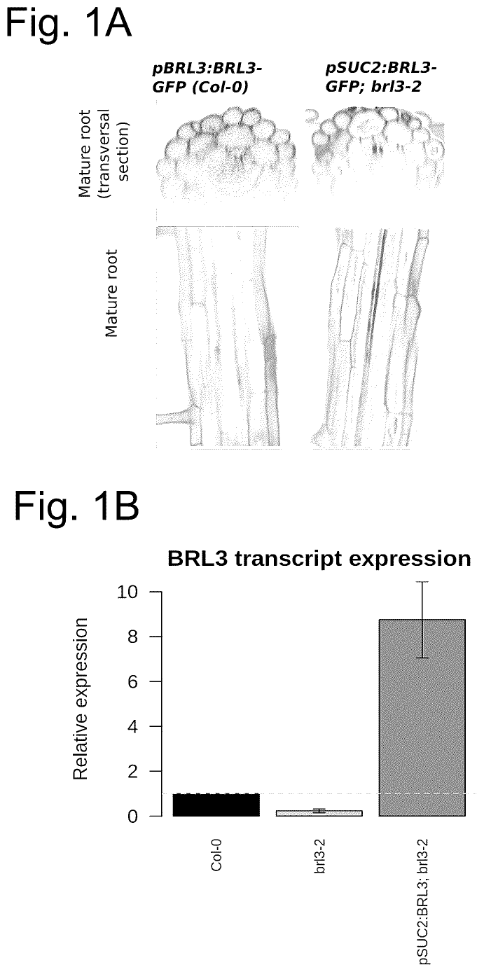

To investigate the function of BRL3 in phloem cells, we expressed BRL3 (SEQ ID NO 1, under the phloem companion cell specific promoter SUC2 (SEQ ID NO 13) in a loss-of-function mutant background (M3-2), resulting in plants expressing BRL3 receptor uniquely in the phloem companion cells (pSUC2:BRL3-GFP; brl3-2 lines, FIG. 1A). RT-qPCR analysis revealed that this strategy actually causes a tissue-specific BRL3 overexpression (FIG. 1B)

### Phloem-Specific Expression of BRL3 Enhances Plant Growth Traits

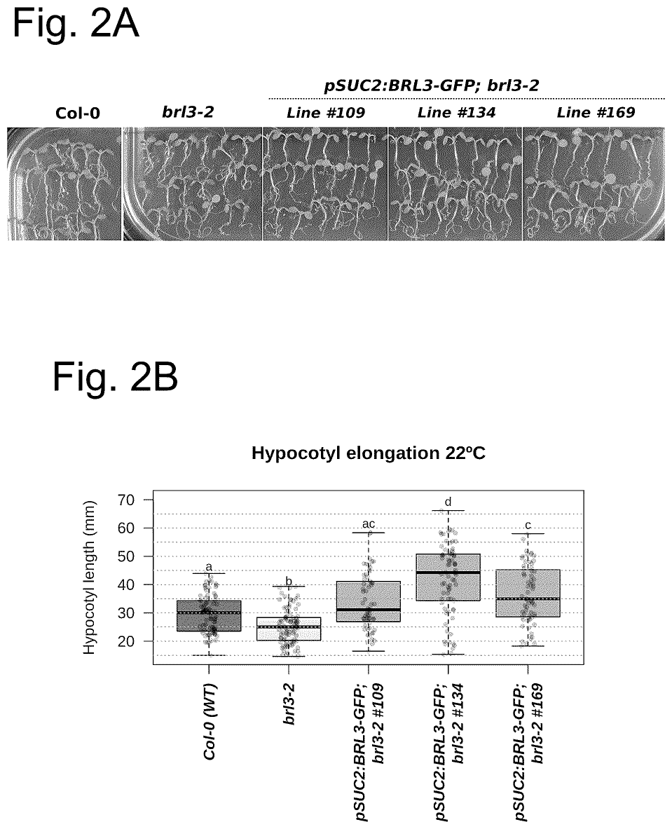

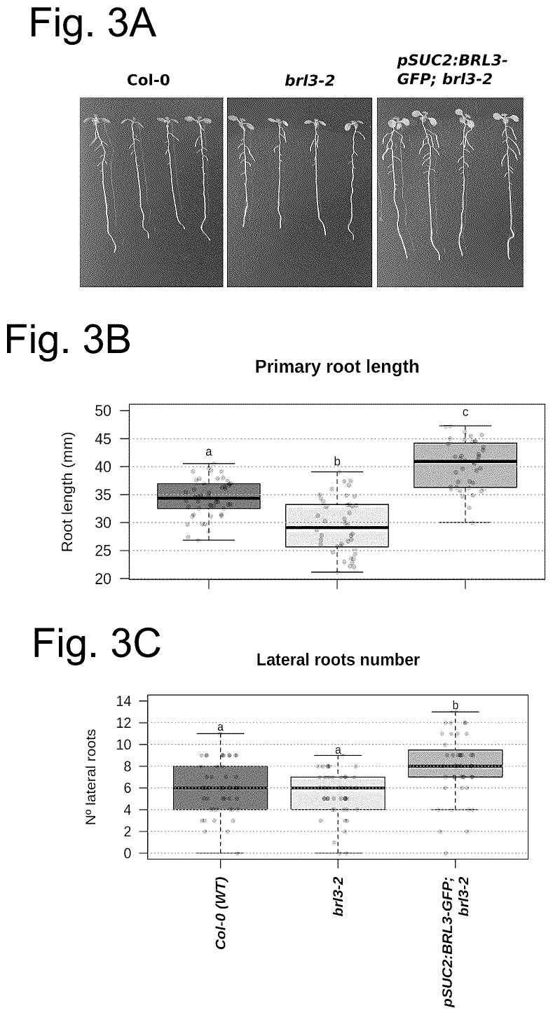

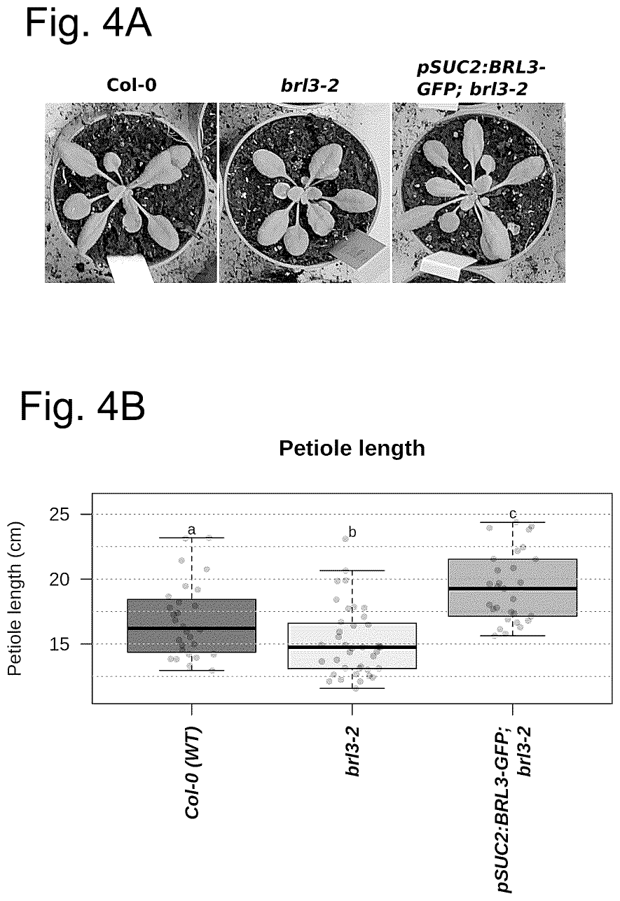

The analysis of pSUC2:BRL3-GFP; brl3-2 seedlings show total recovery of brl3-2 hypocotyl growth defects and even its enhancement. These effects were observed in three independent transgenic lines (FIG. 2A, B). Hereinafter all experiments refer to pSUC2:BRL3-GFP; brl3-2 #134 line. The same vascular-driven growth recovery and enhancement effects were recapitulated in terms of primary root length (FIG. 3A, B), lateral root numbers (FIG. 3C) and bigger rosettes and elongated petioles in adult stage (FIG. 4A, B).

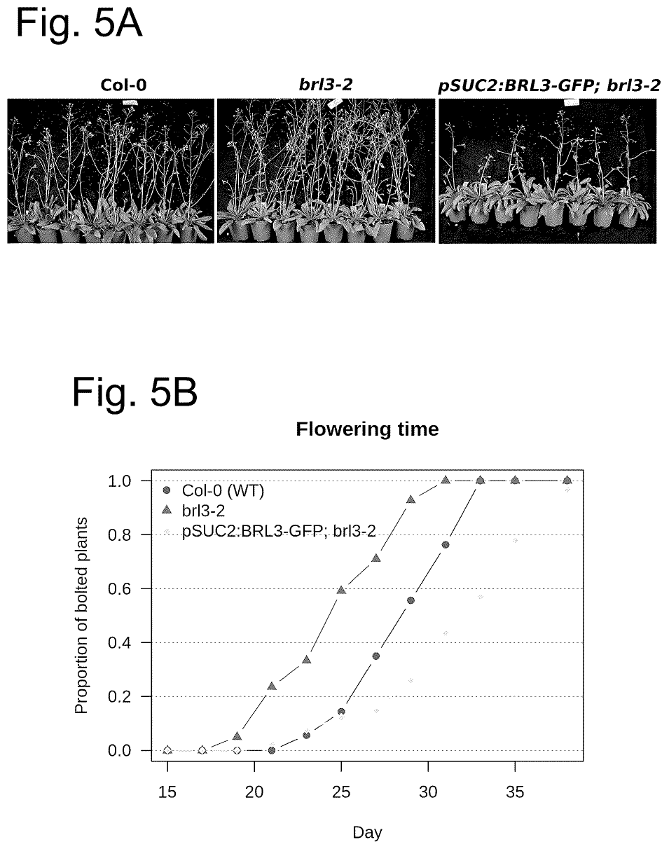

In addition, the pSUC2:BRL3-GFP; brl3-2 lines show significantly delayed transition from vegetative to reproductive phase as reflected by the flowering time analysis, contrary to the loss-of-function mutant brl3-2, which has an accelerated flowering transition (FIG. 5). This observation reveals that the action of BRL3 in the phloem is enough to affect plant flowering process.

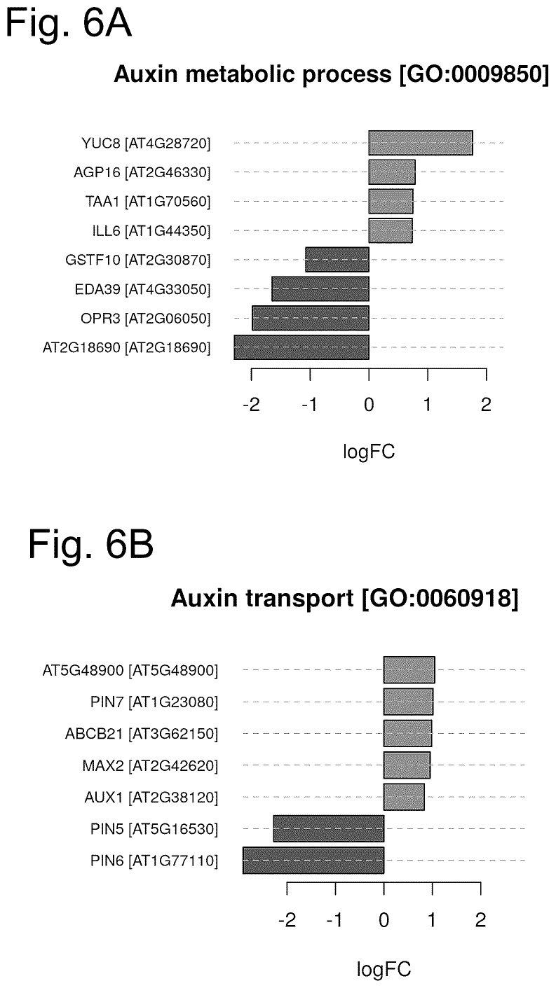

Transcriptome comparison (RNAseq analysis) of pSUC2:BRL3-GFP; brl3-2 hypocotyls versus brl3-2 hypocotyls revealed 644 genes being affected due to the BRL3 expression specifically in the phloem. Among these, a significant part was involved in responses to the phytohormone auxin, which is well-known to drive growth and developmental responses (Mroue et al., 2018; Zhao, 2018). This observation is in accordance with the growth effects observed in pSUC2:BRL3-GFP; brl3-2 plants. Particularly, many genes annotated in subcategories involving in the synthesis (catabolism) and transport of auxin appeared deregulated, suggesting the capacity of BRL3 signaling in the phloem to affect synthesis and transport of auxin. These genes are deployed in FIG. 6.

### Specific BRL3 Expression in the Phloem Affects Plant Universal Stress Responses

### Specific BRL3 Expression in the Phloem Drive Root Hydrotropic Response

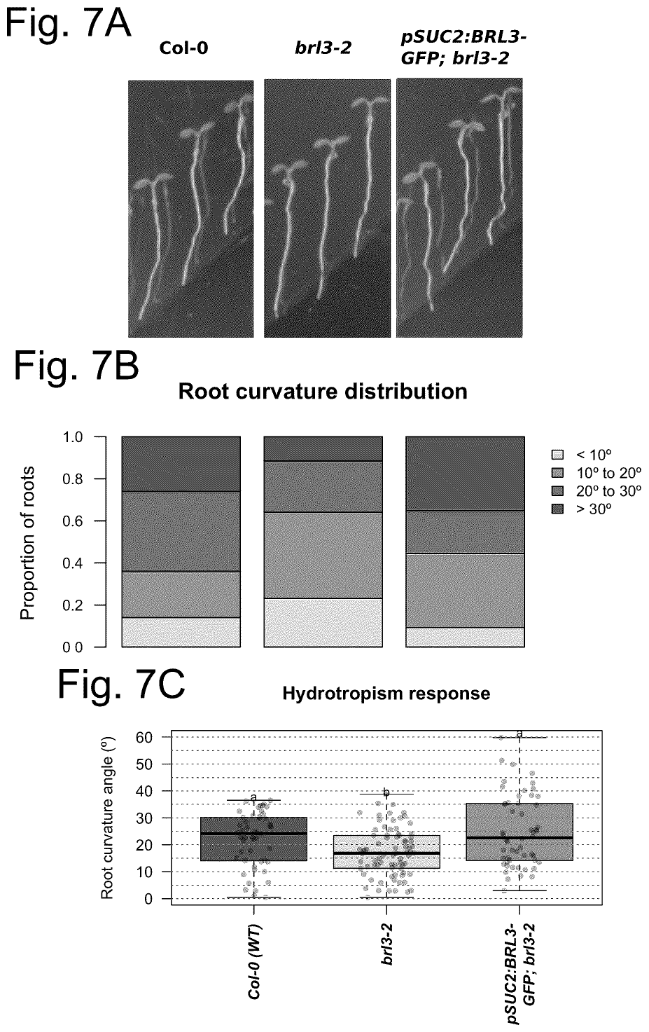

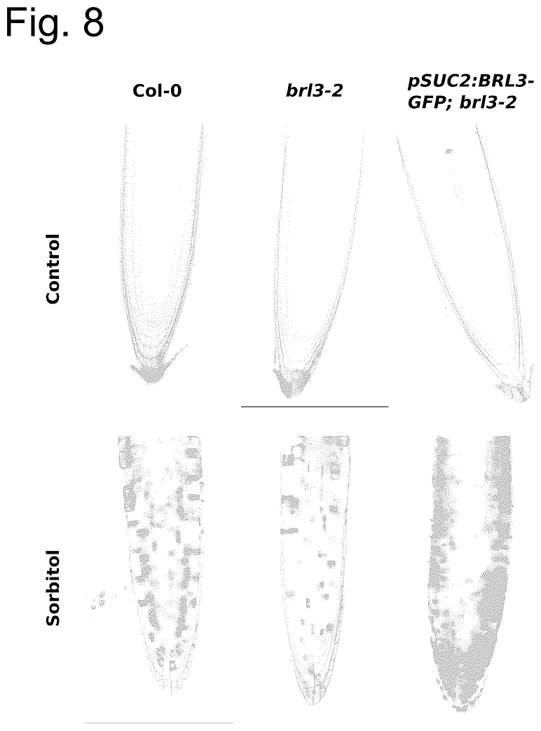

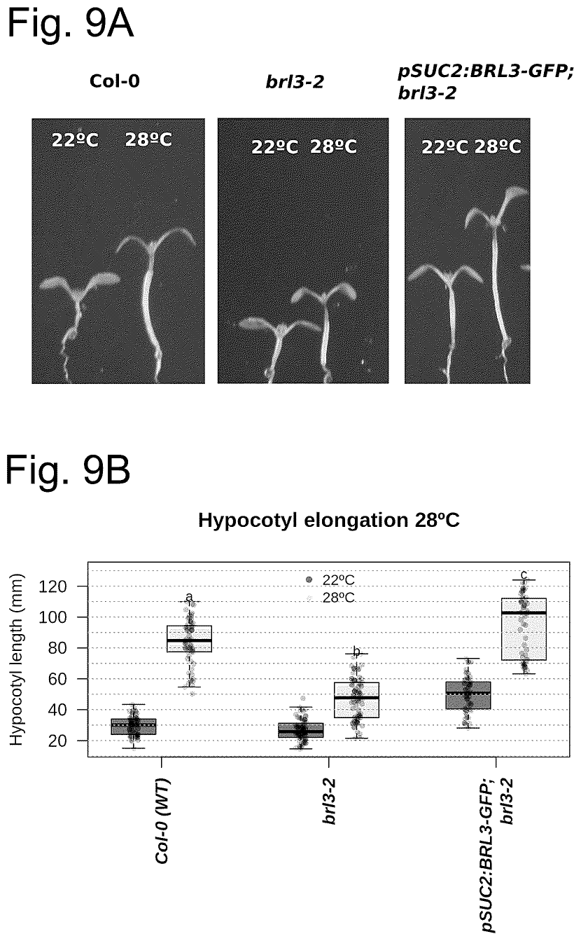

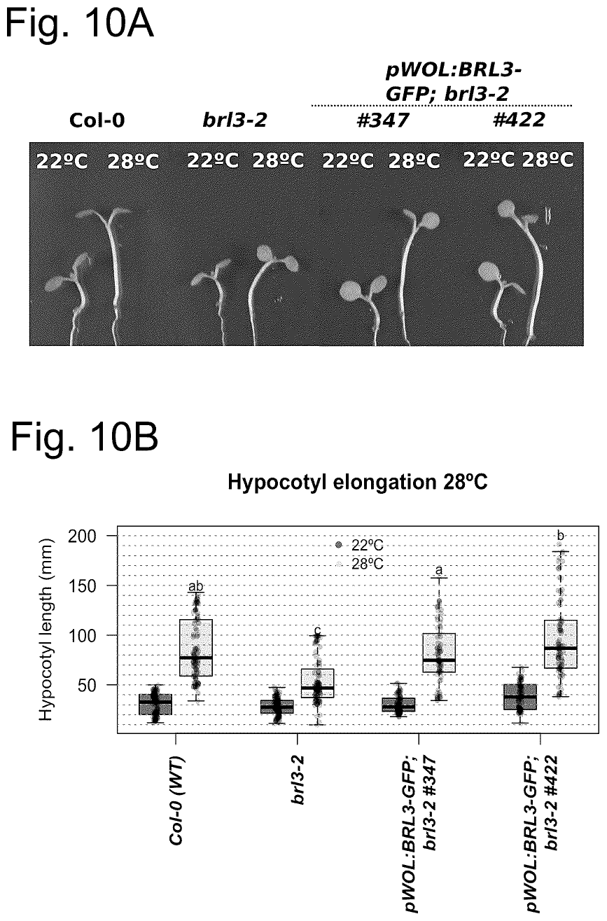

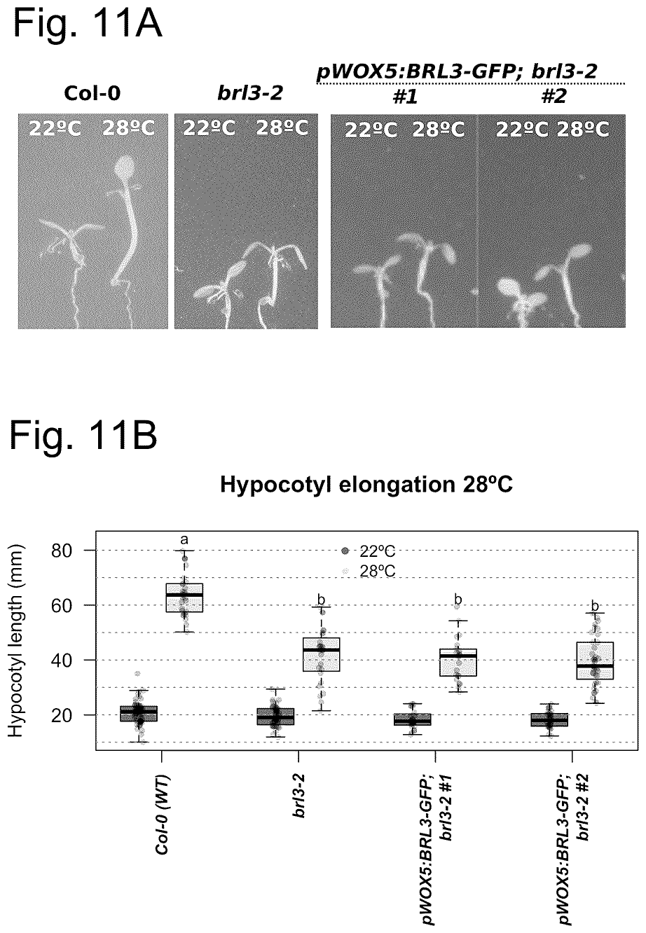

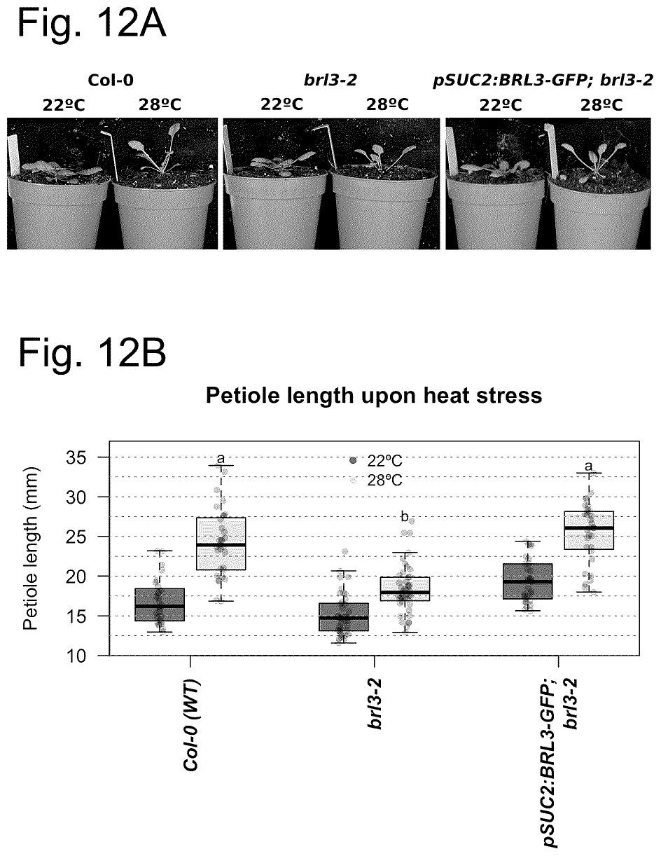

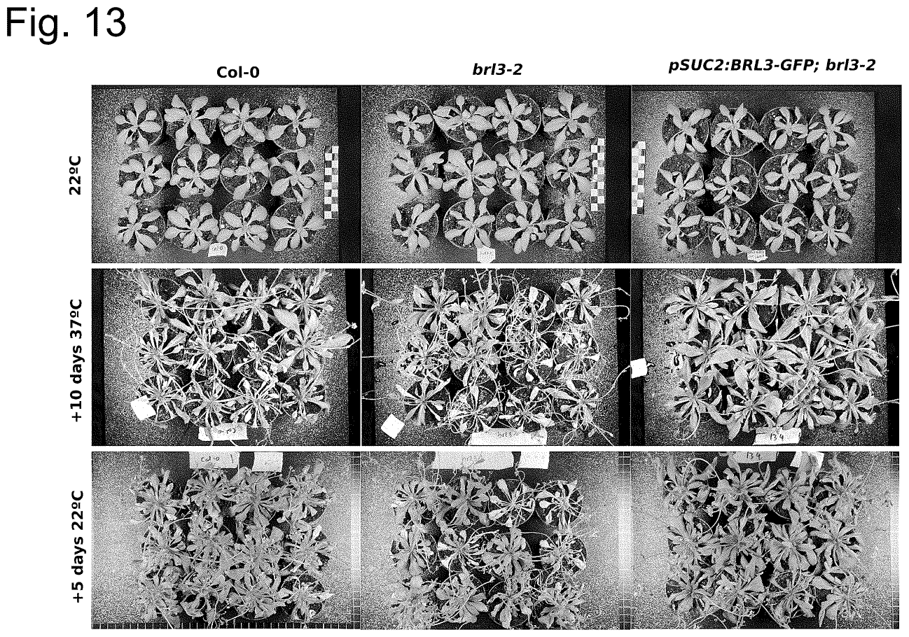

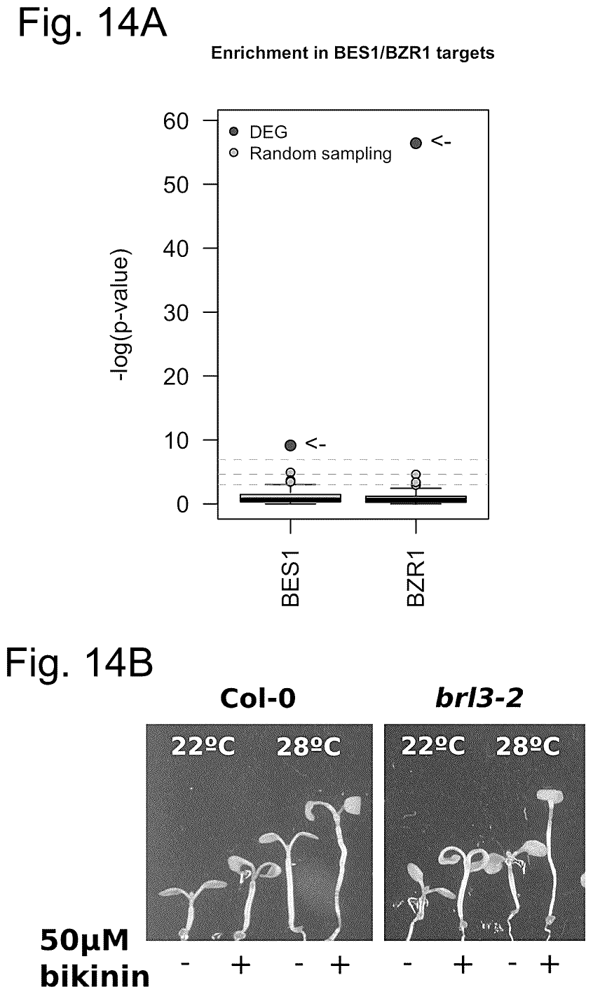

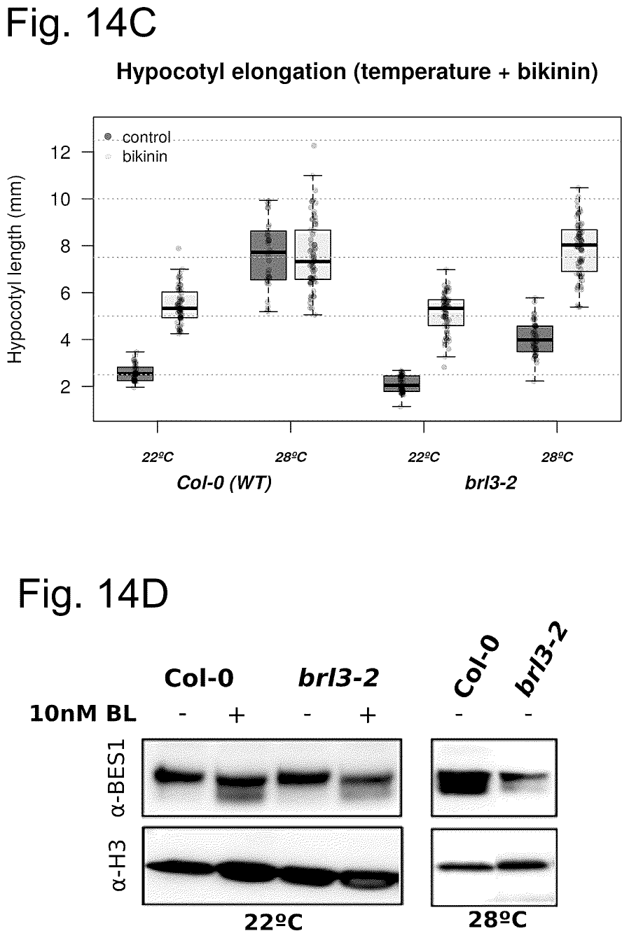

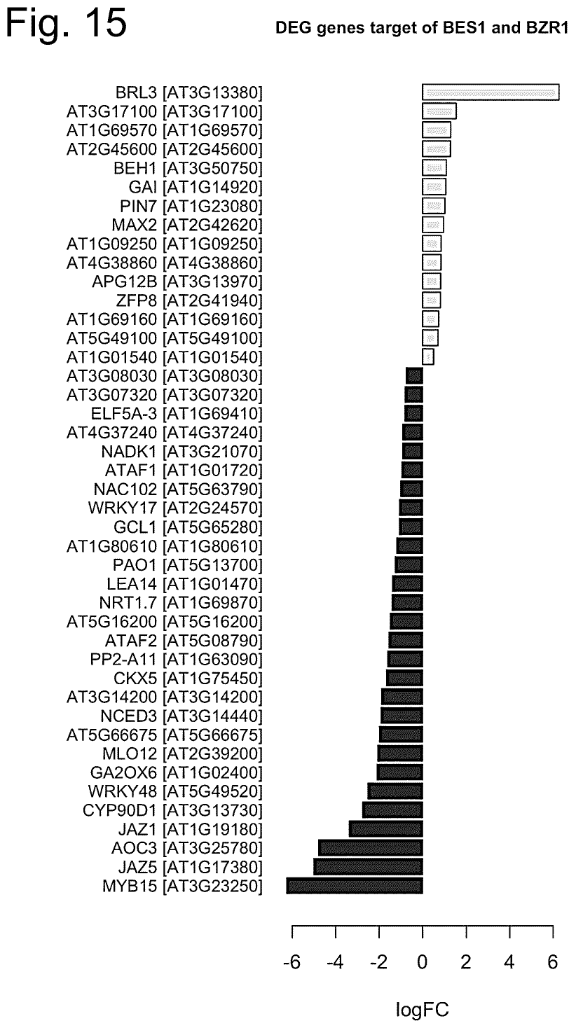

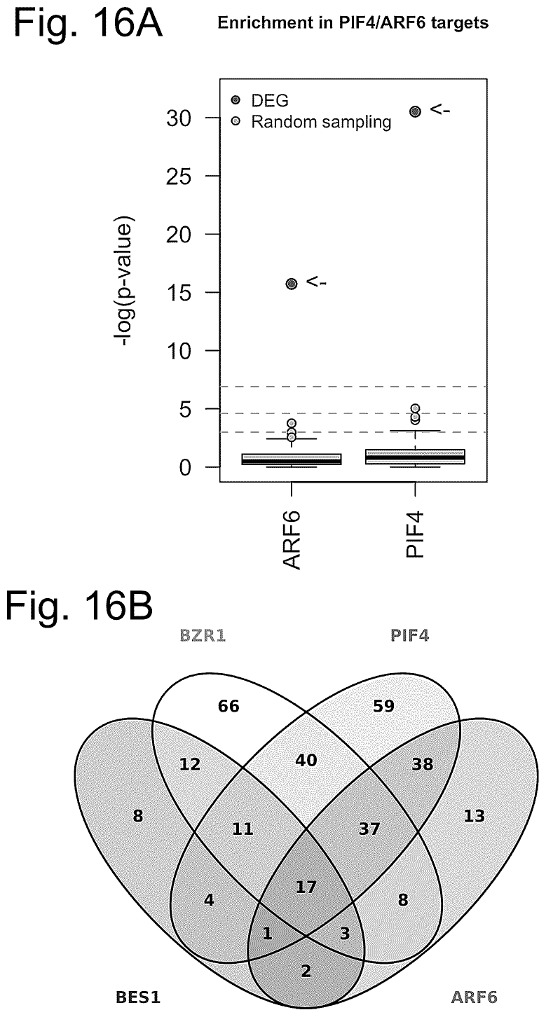

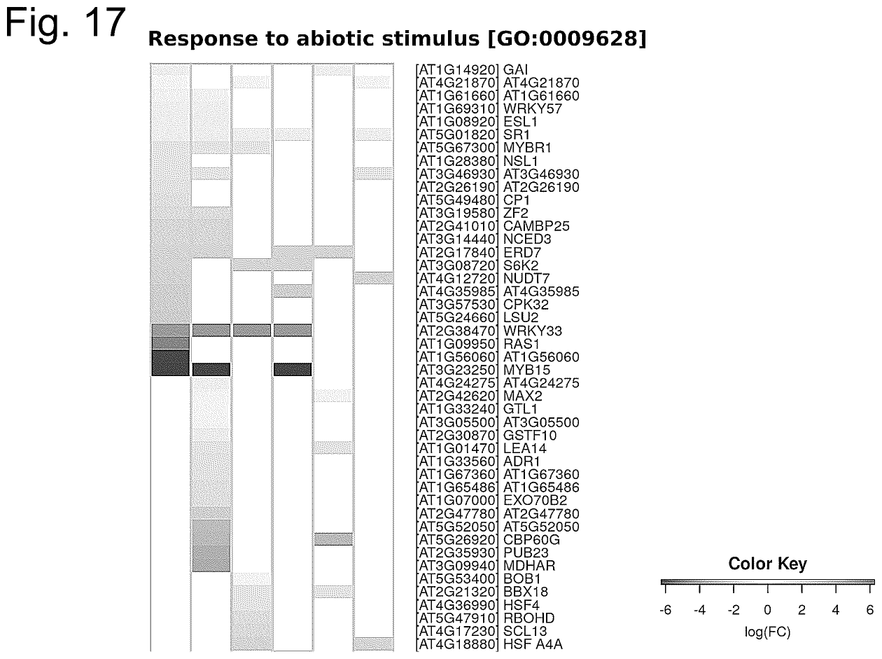

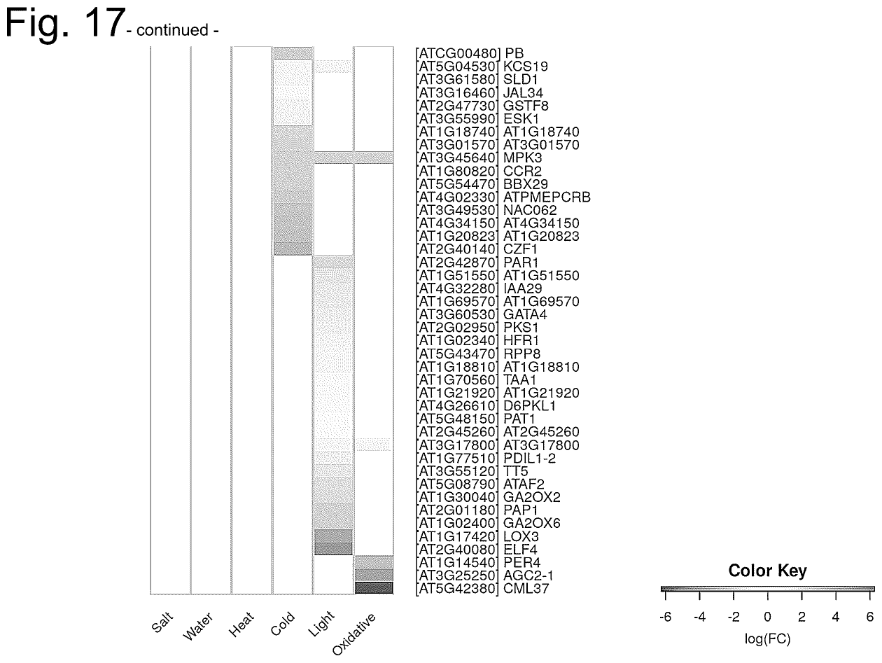

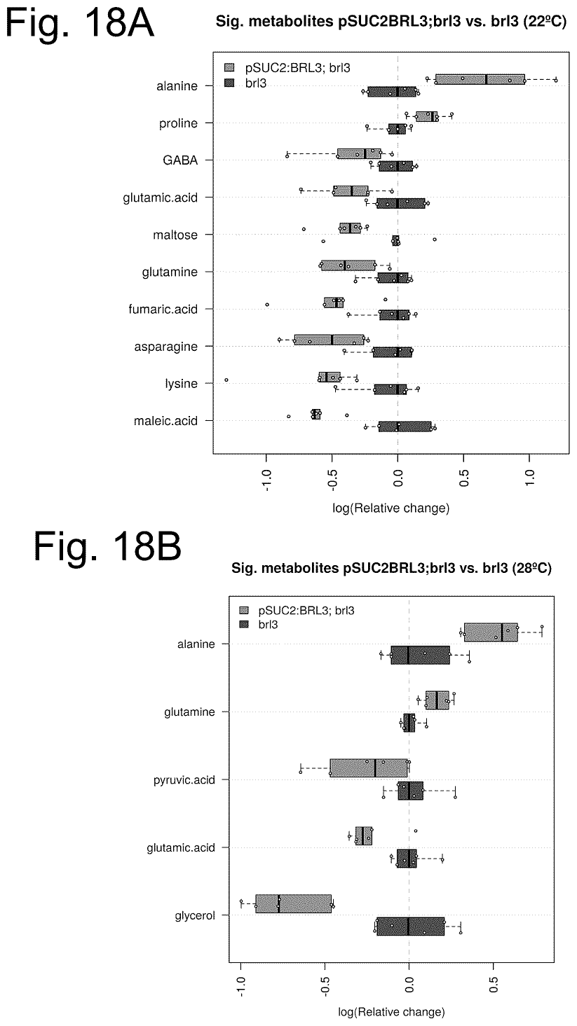

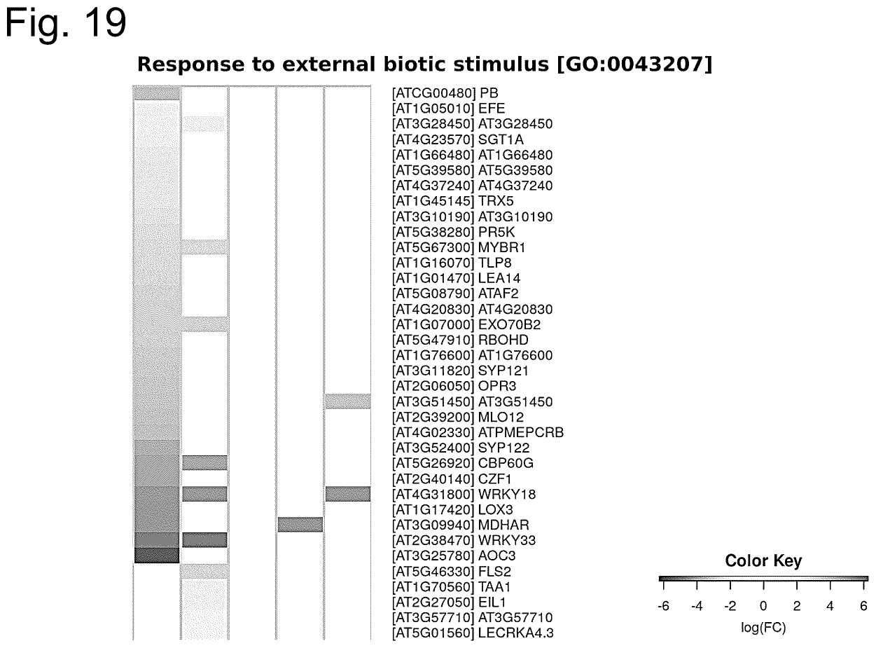

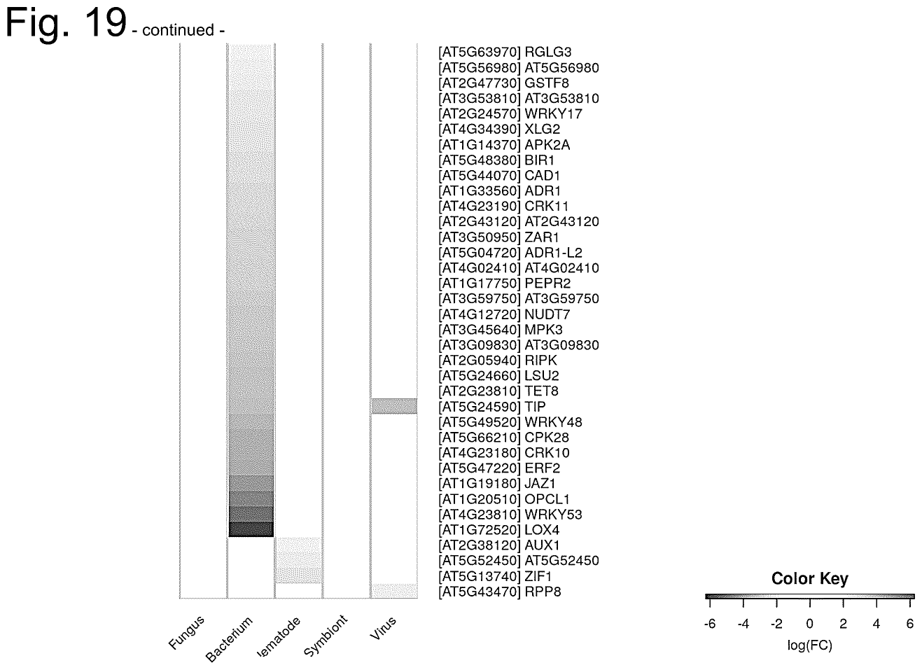

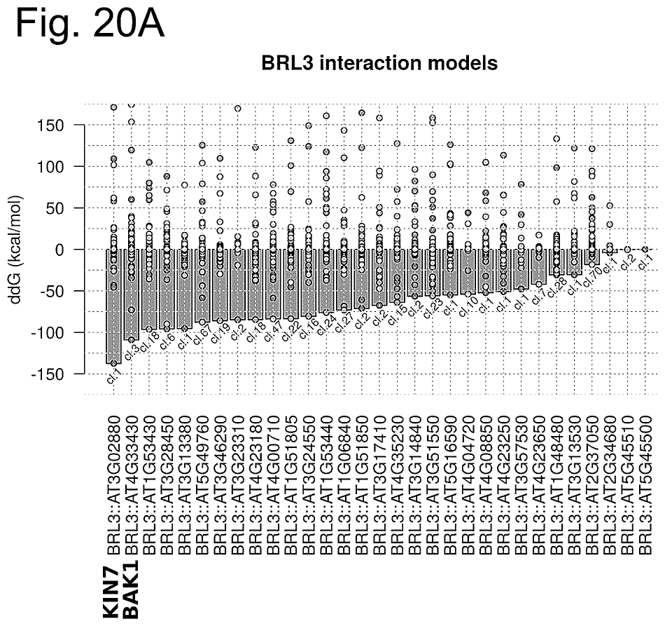

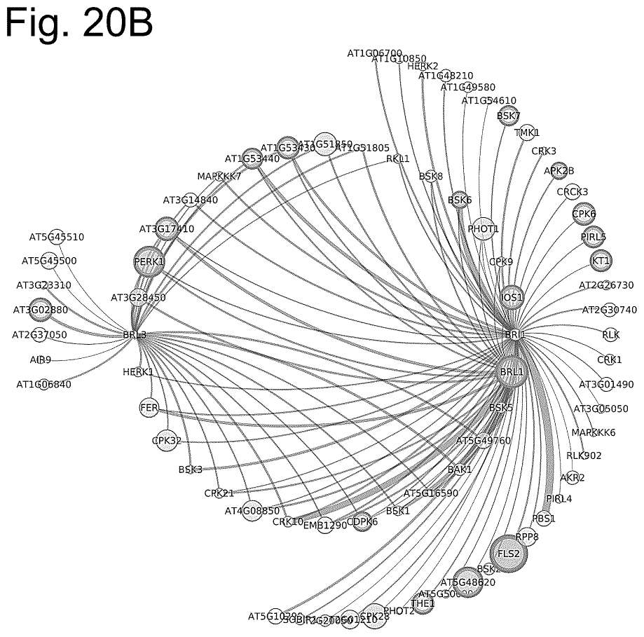

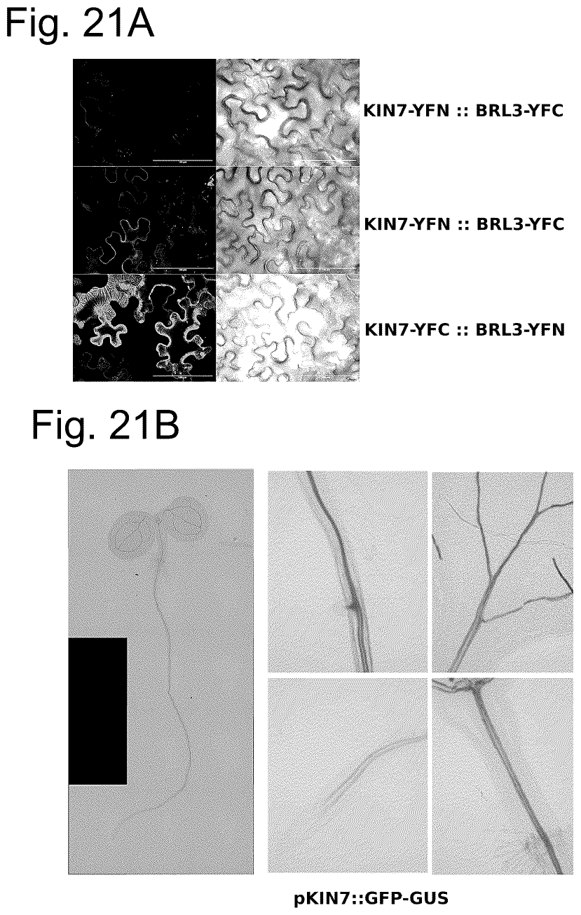

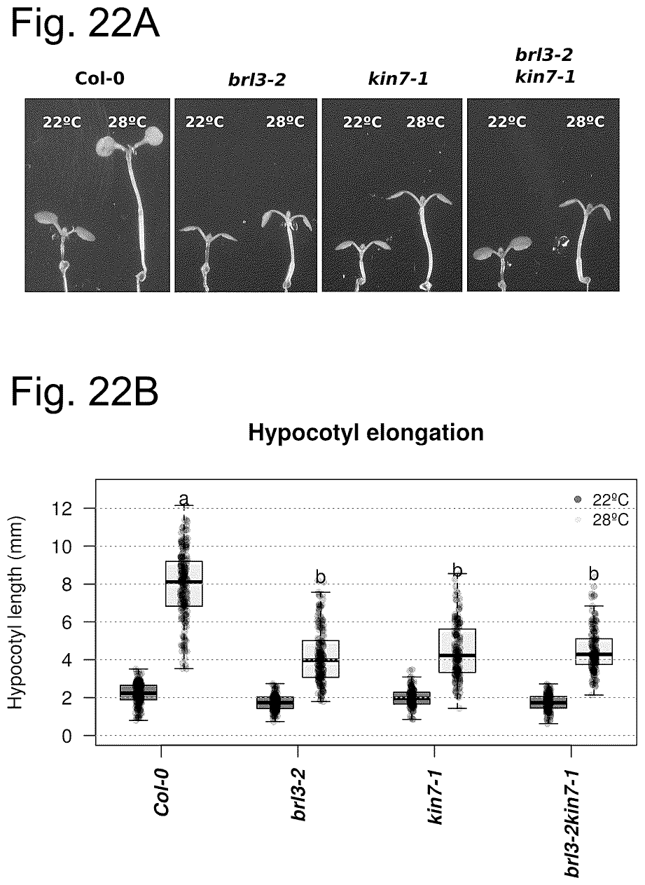

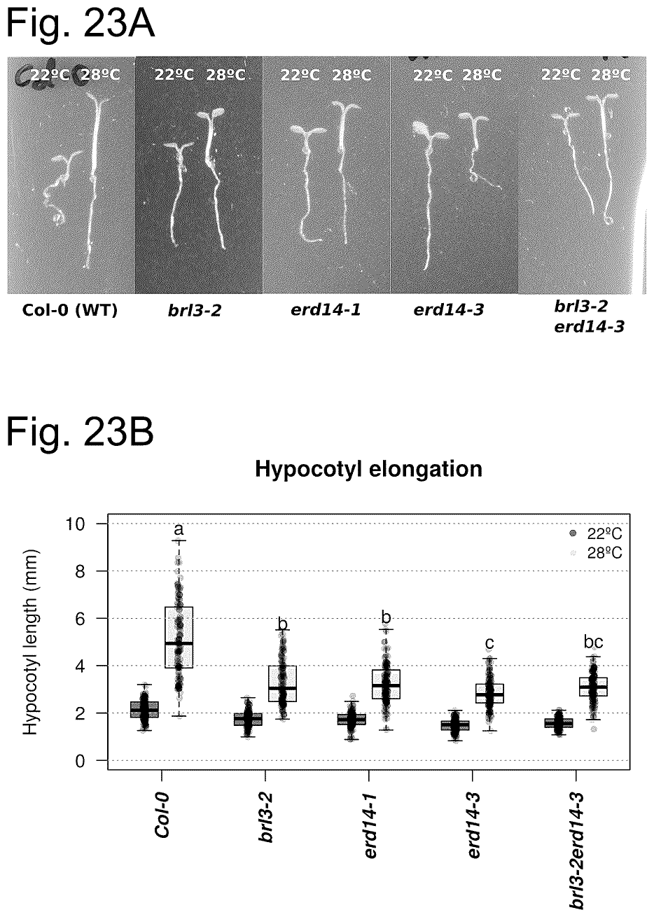

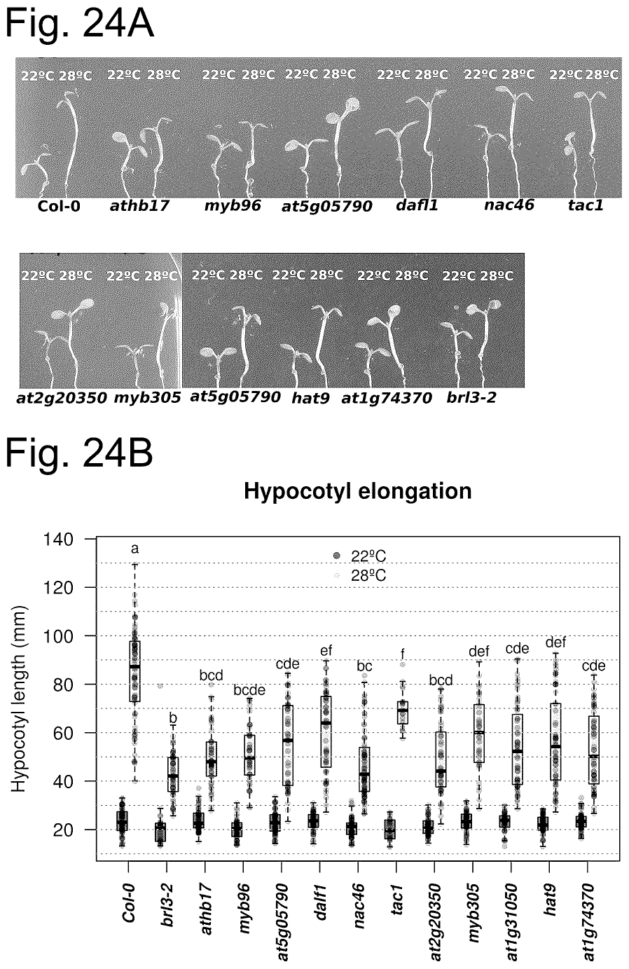

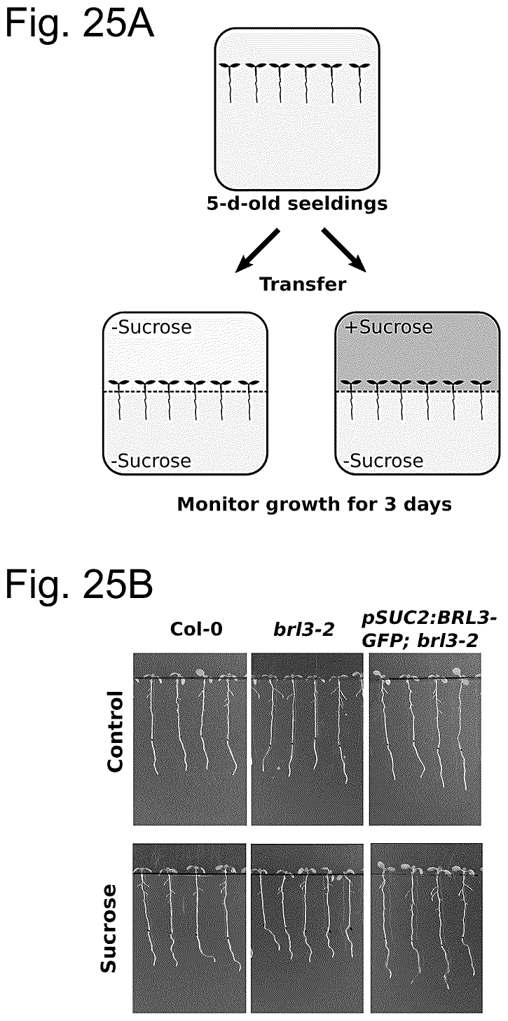

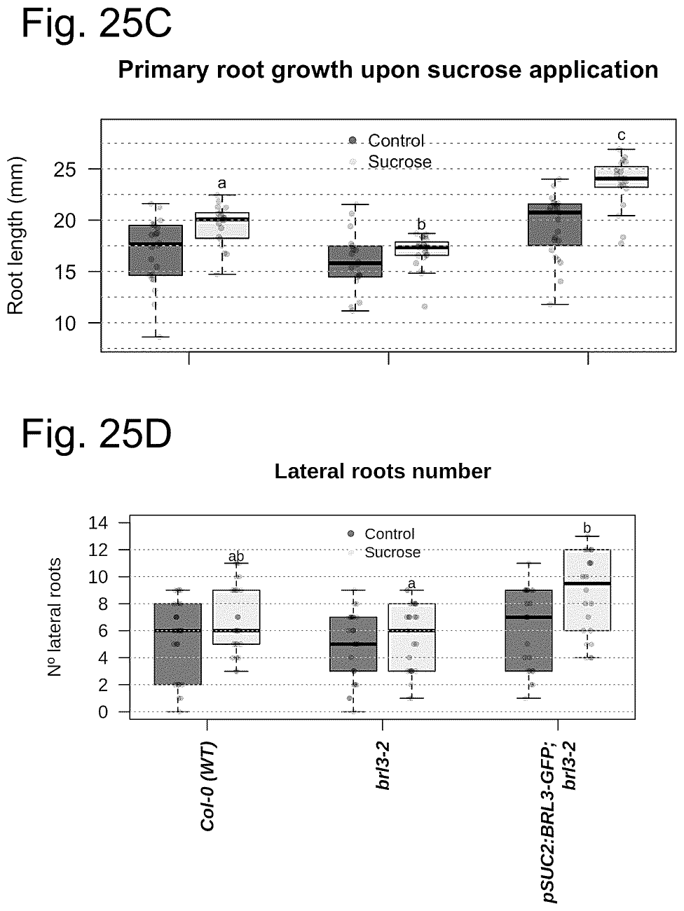

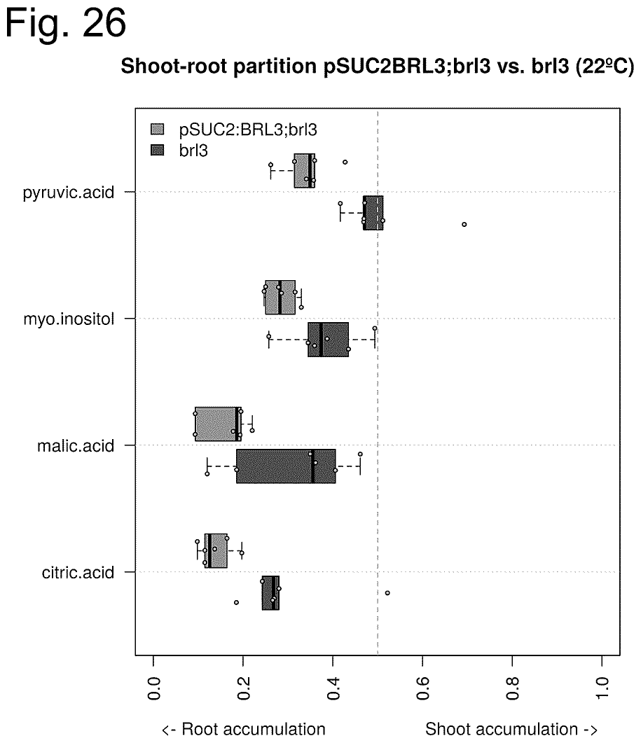

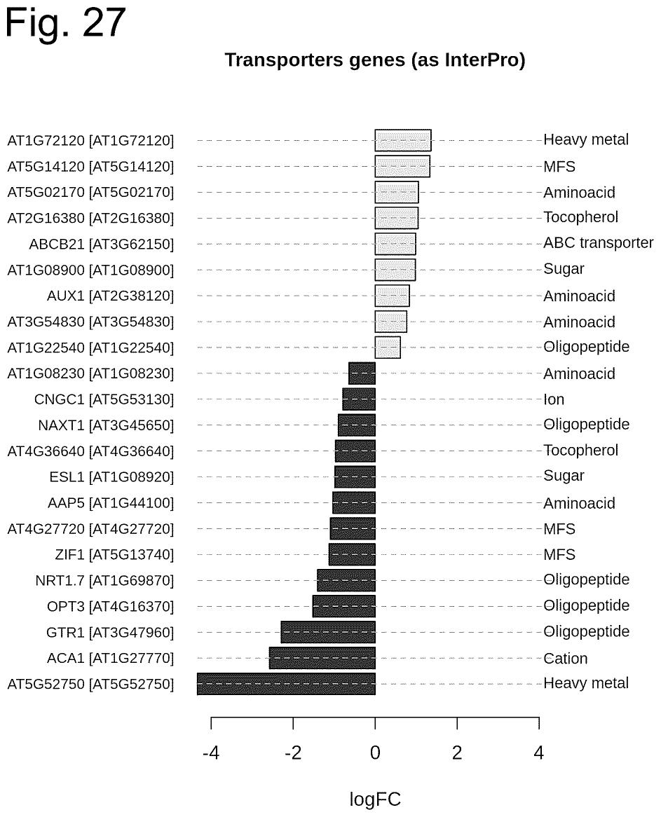

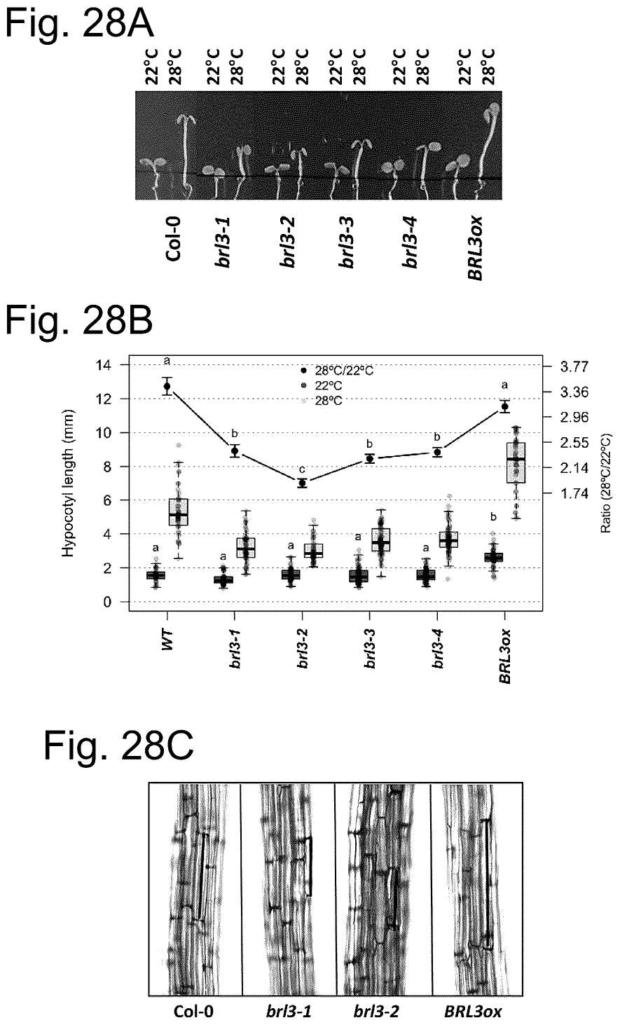

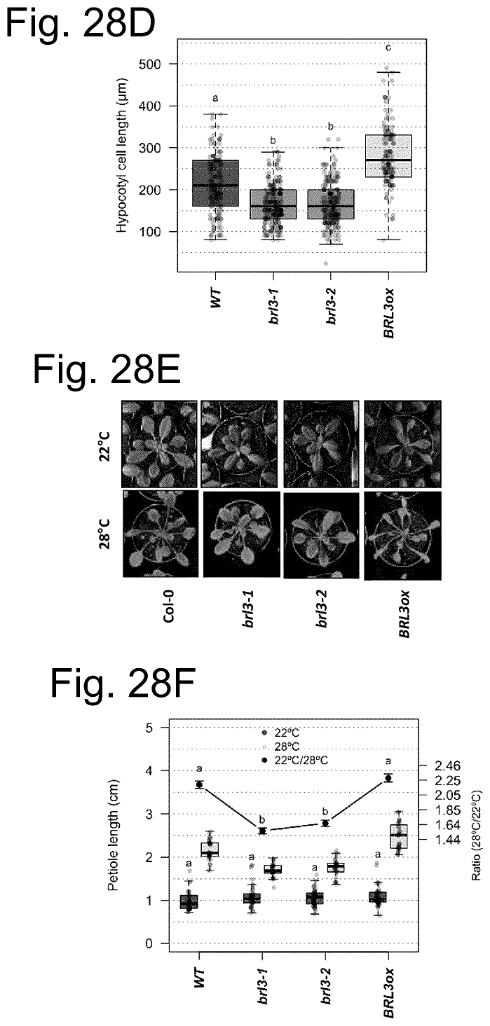

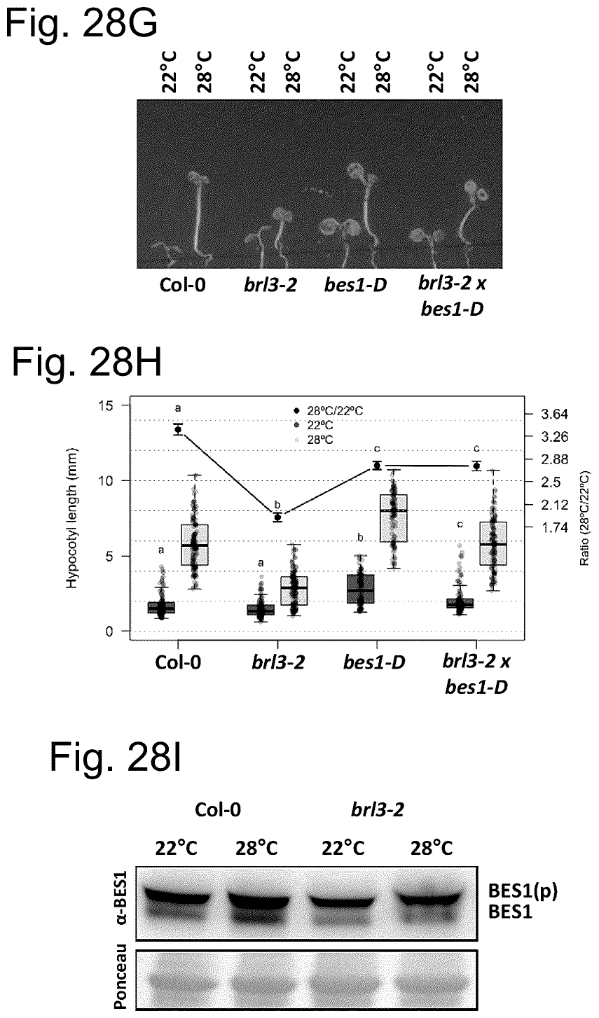

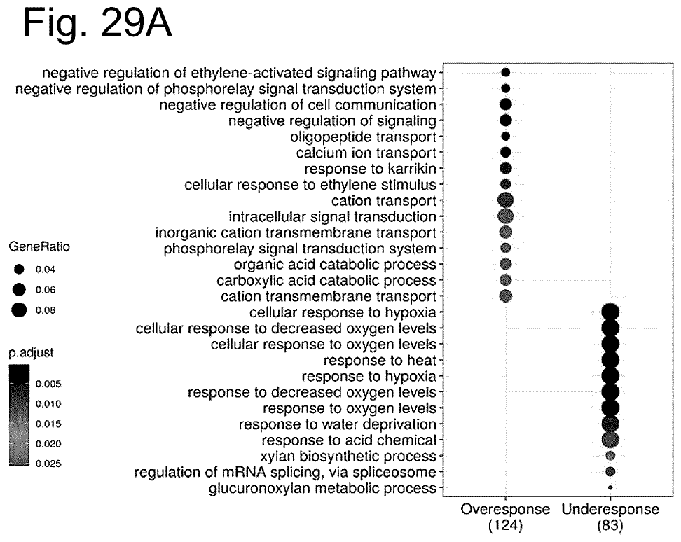

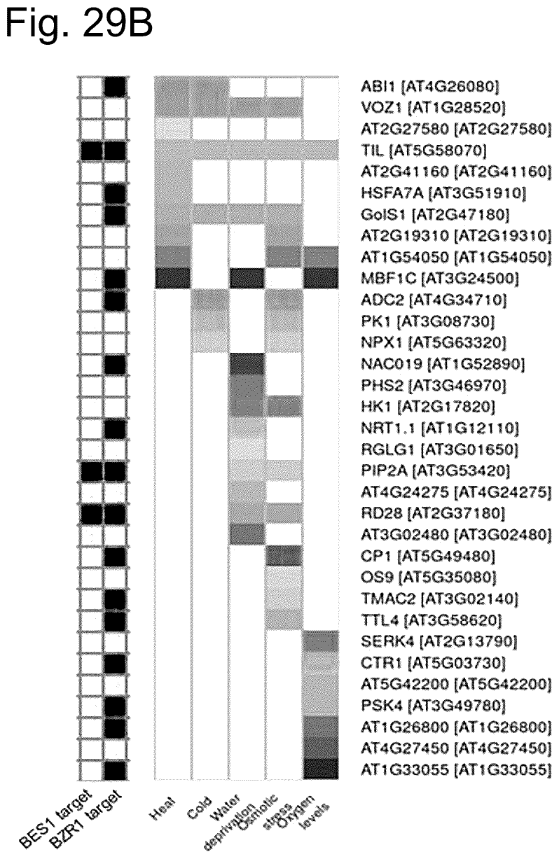

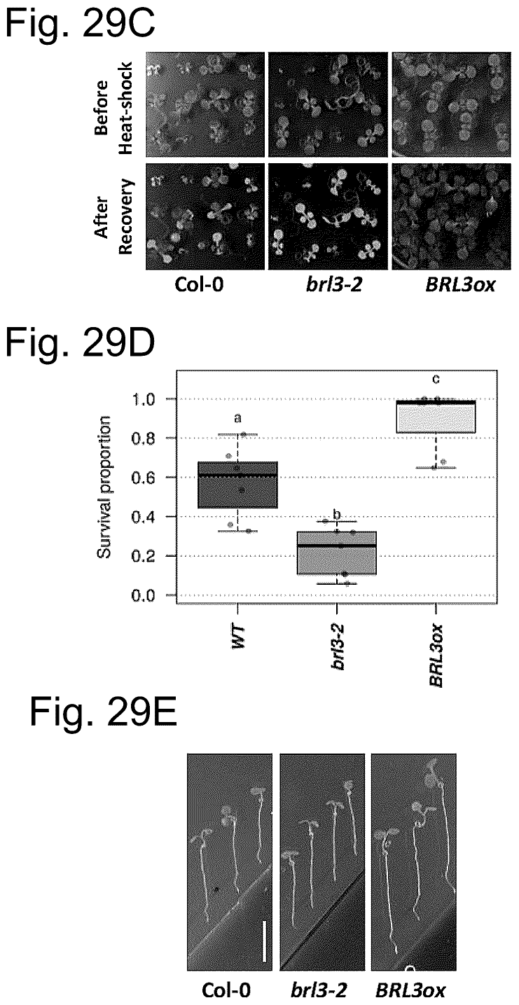

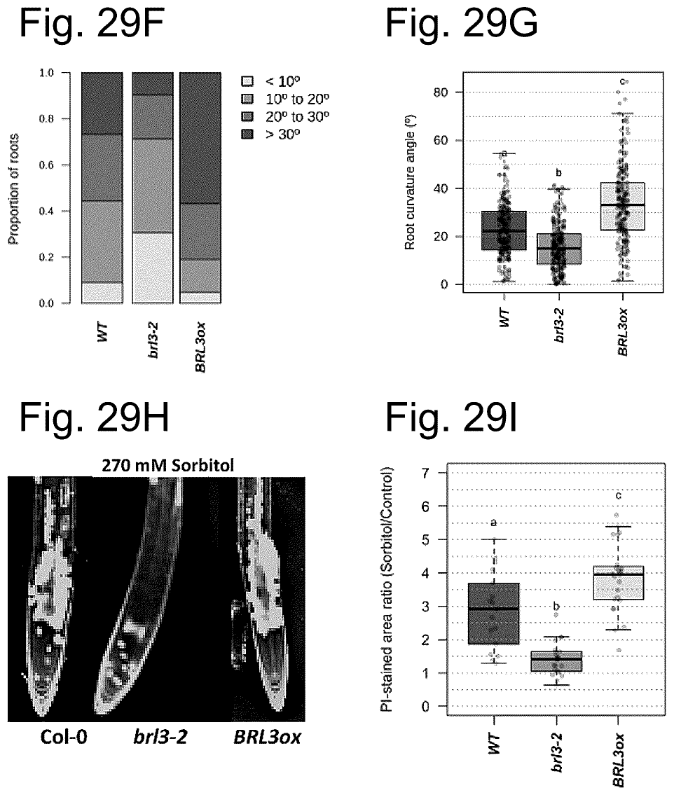

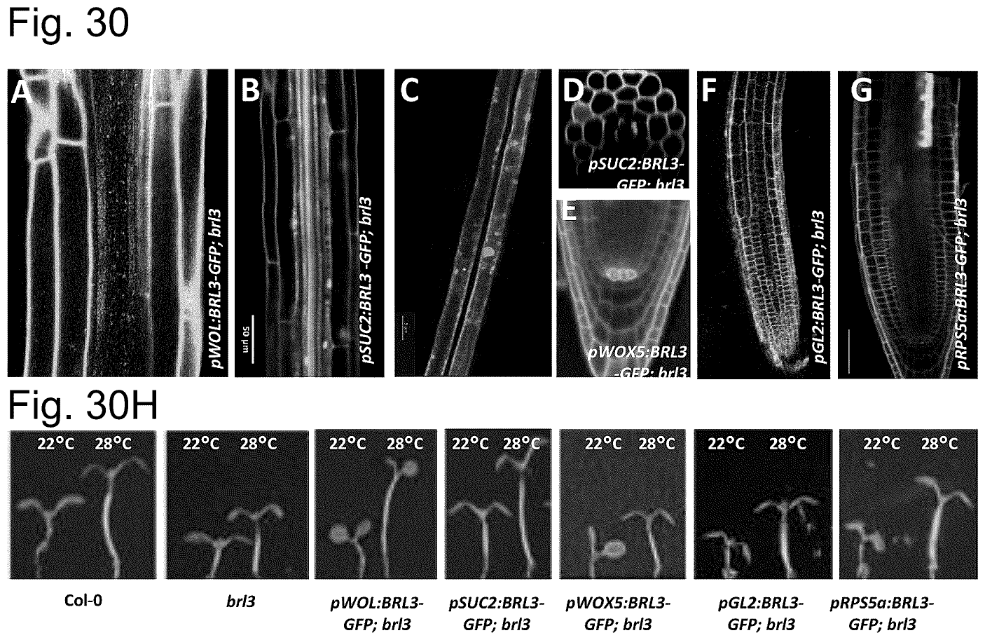

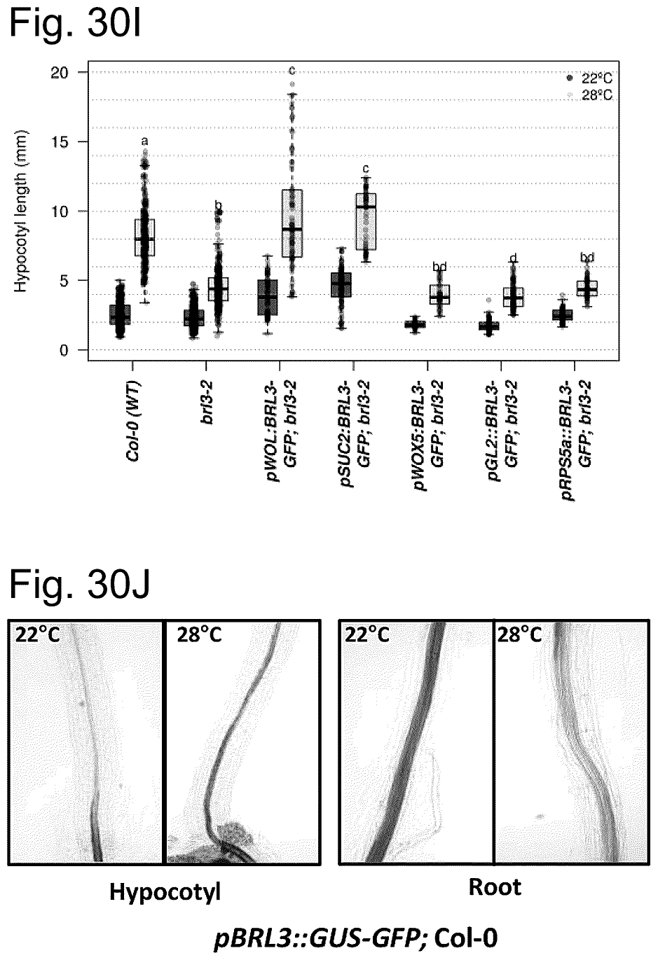

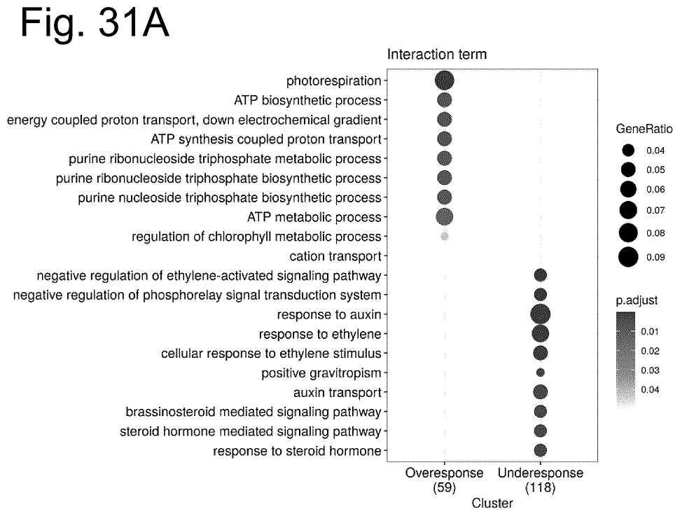

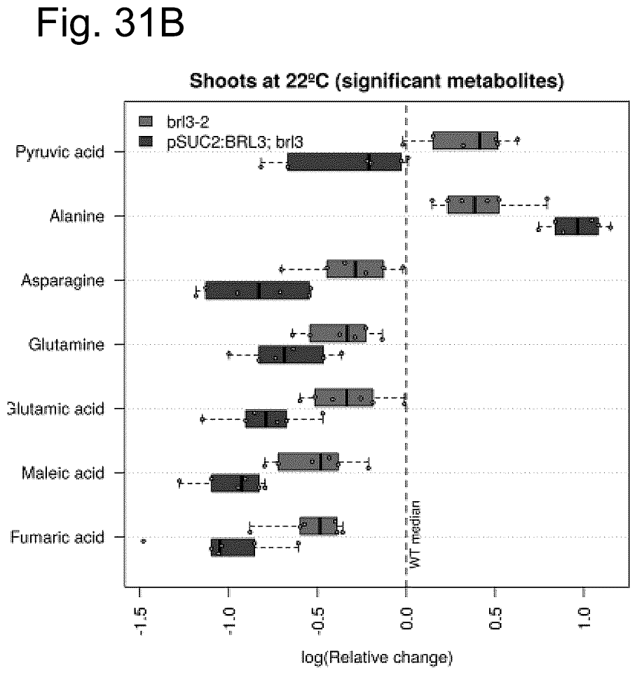

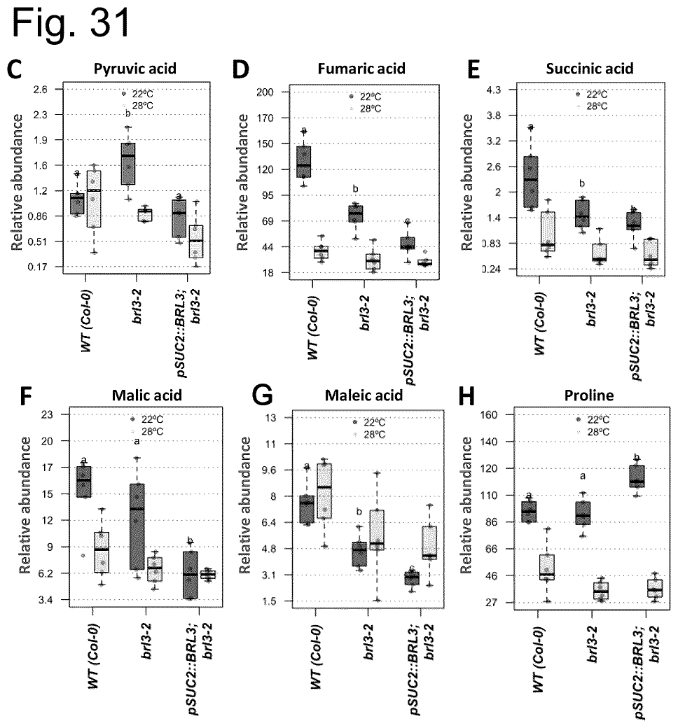

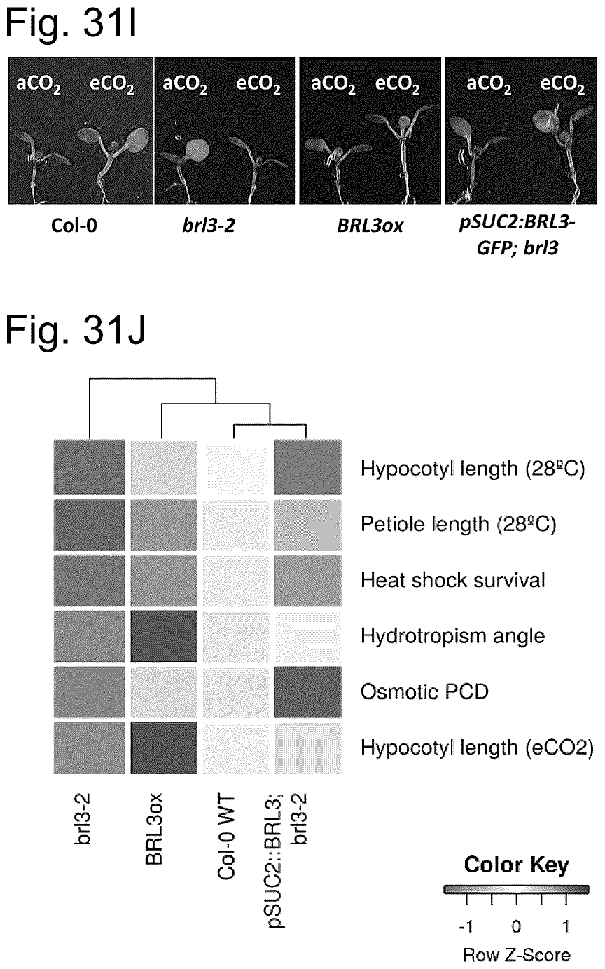

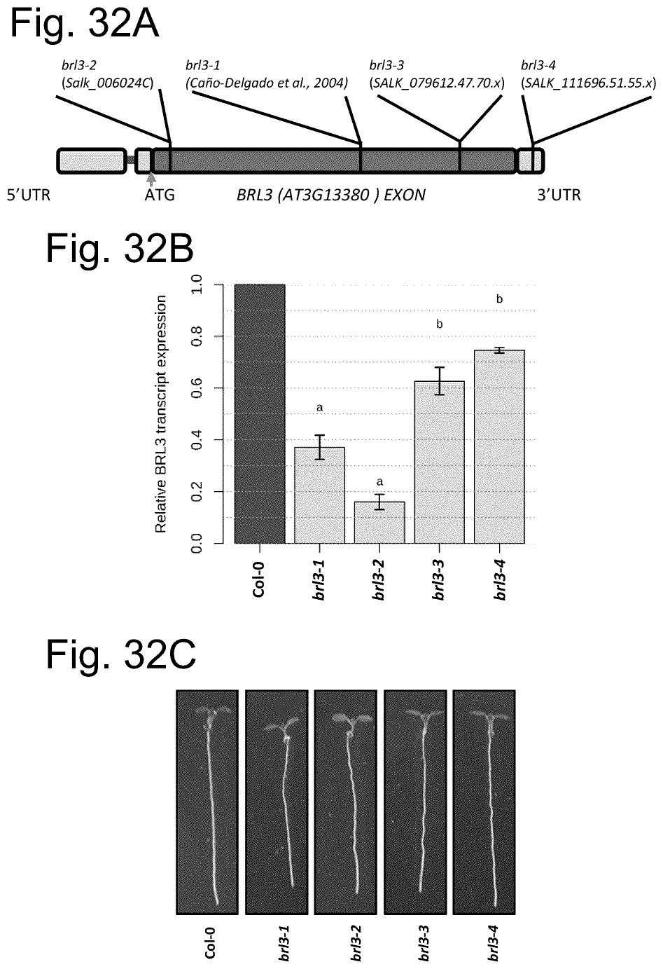

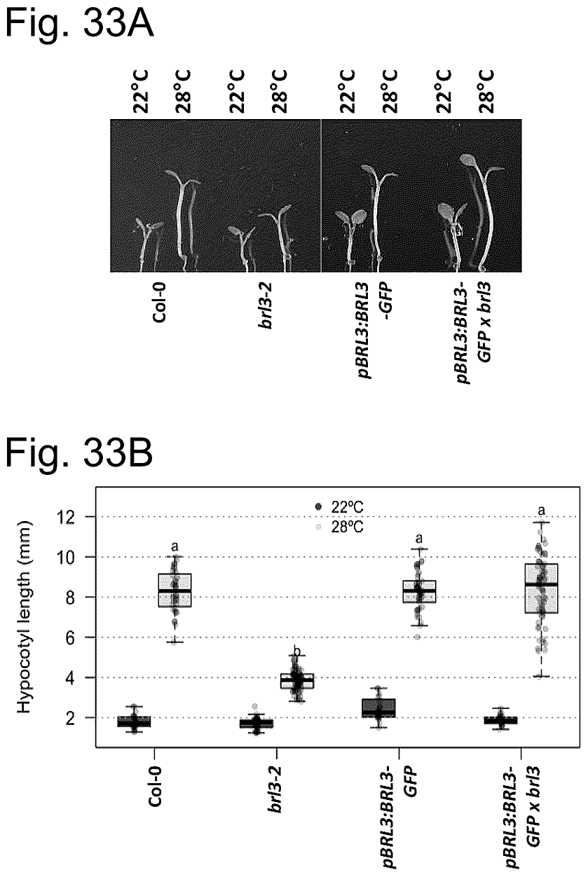

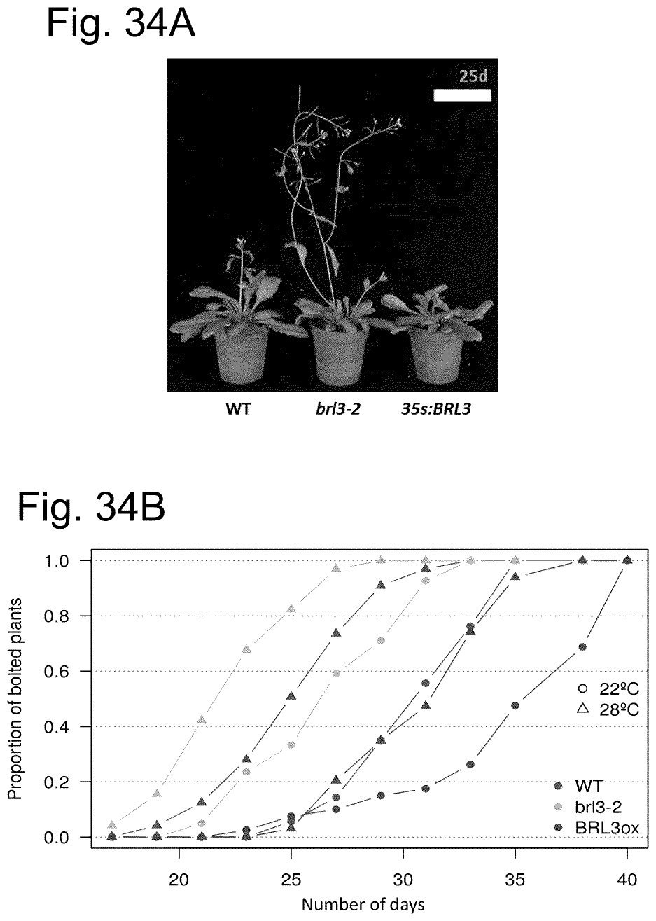

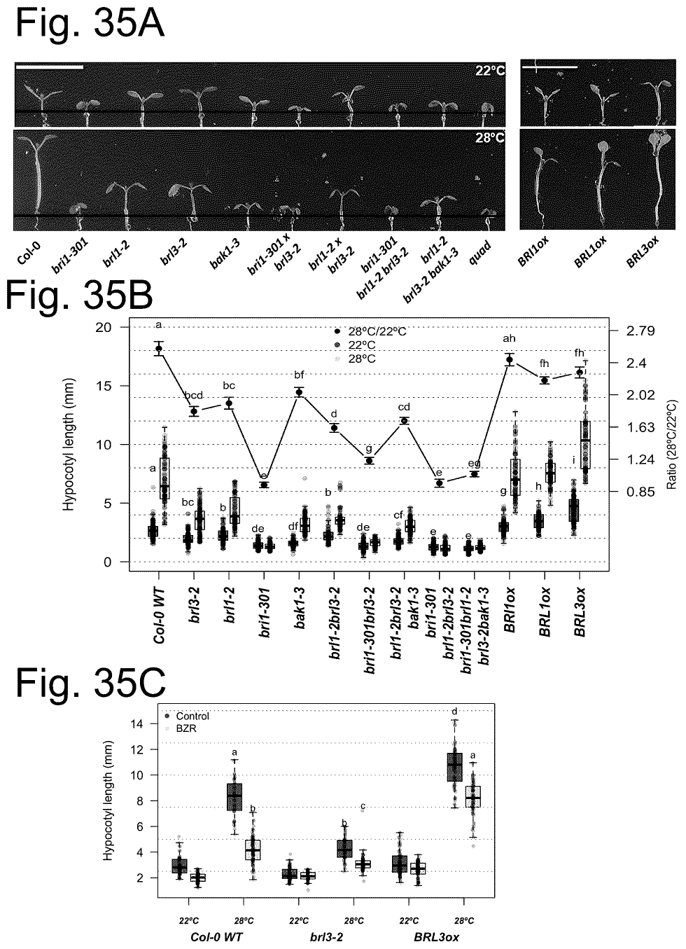

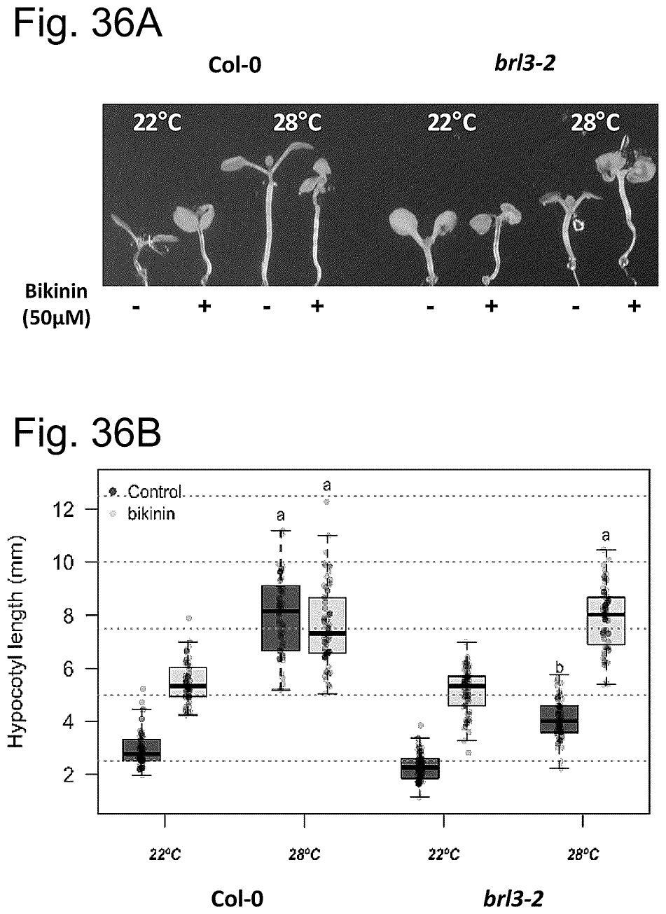

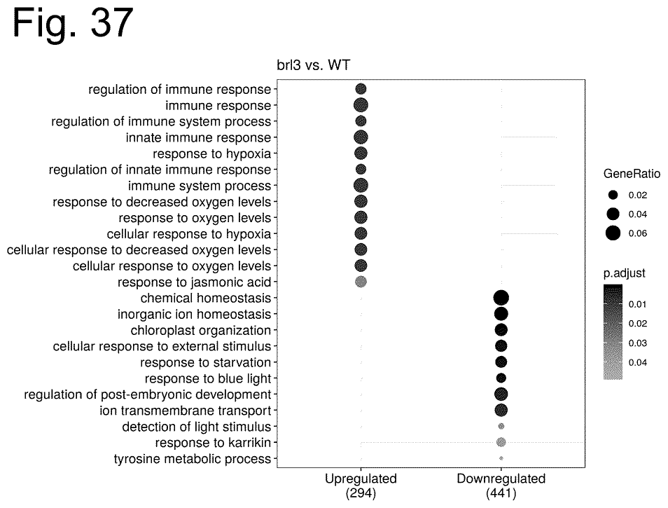

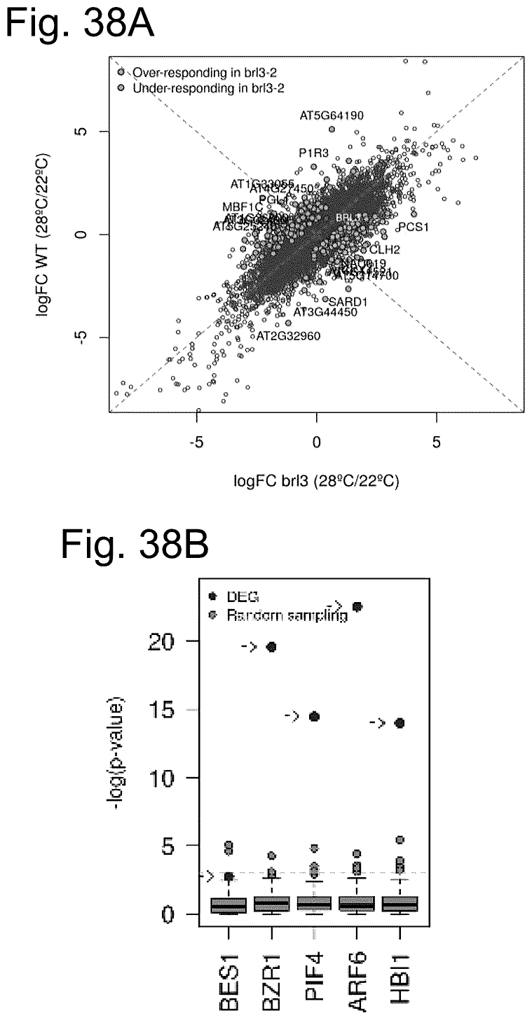

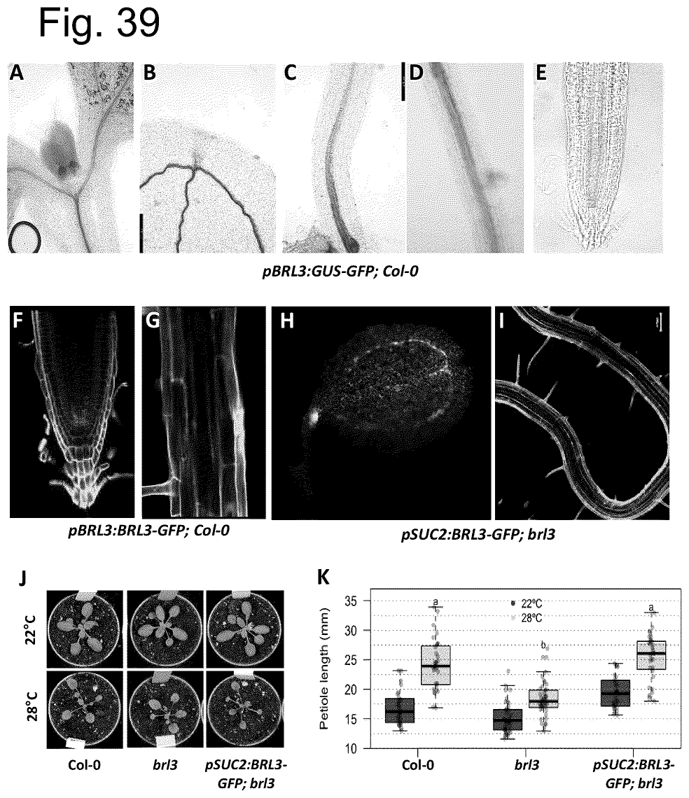

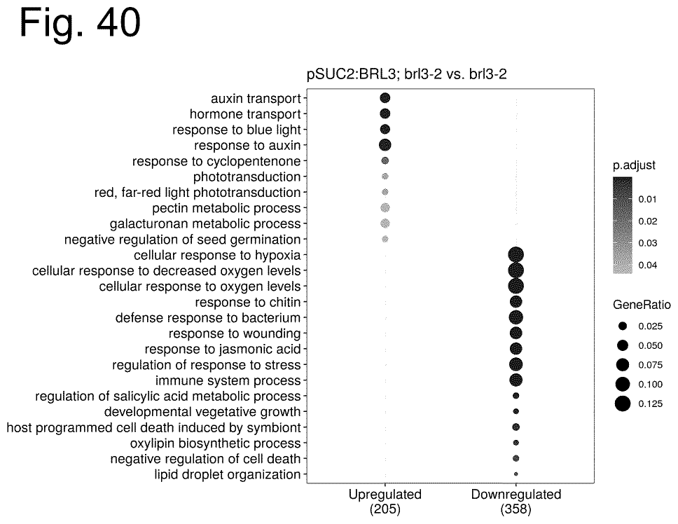

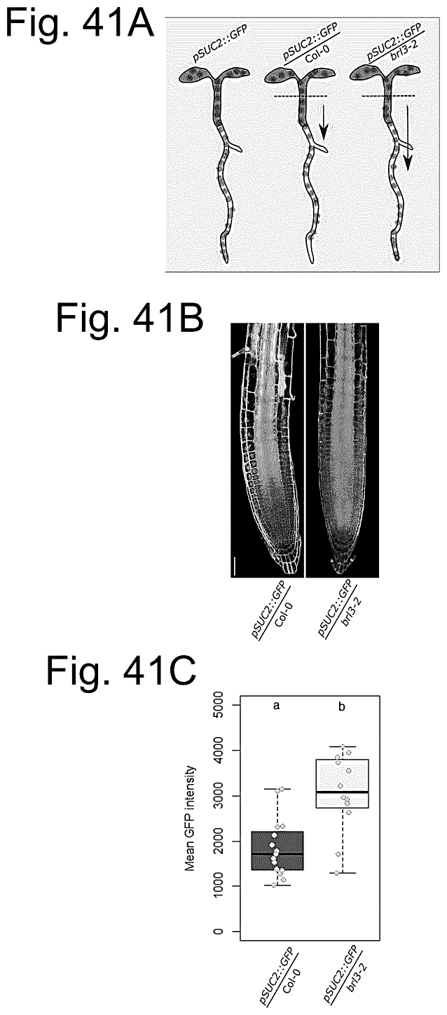

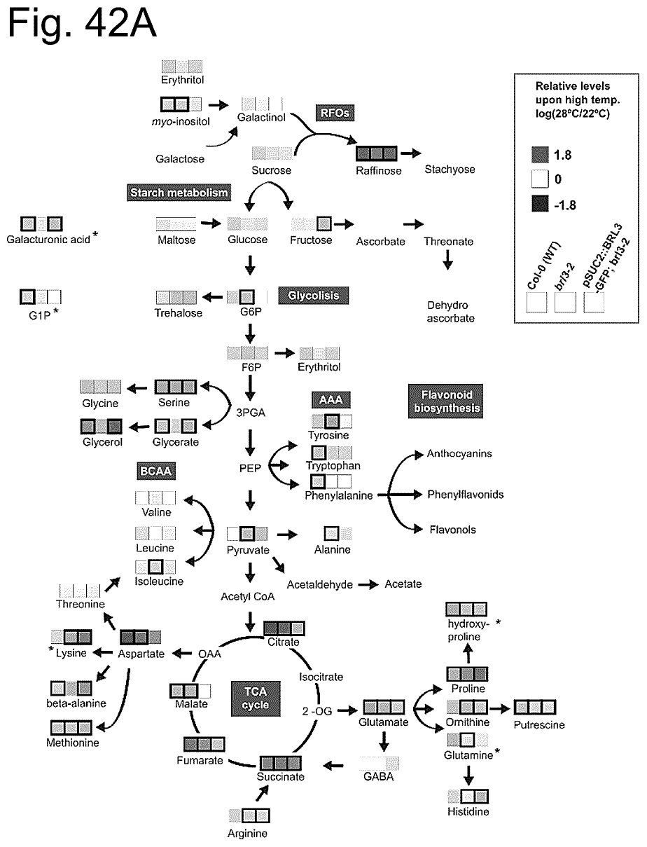

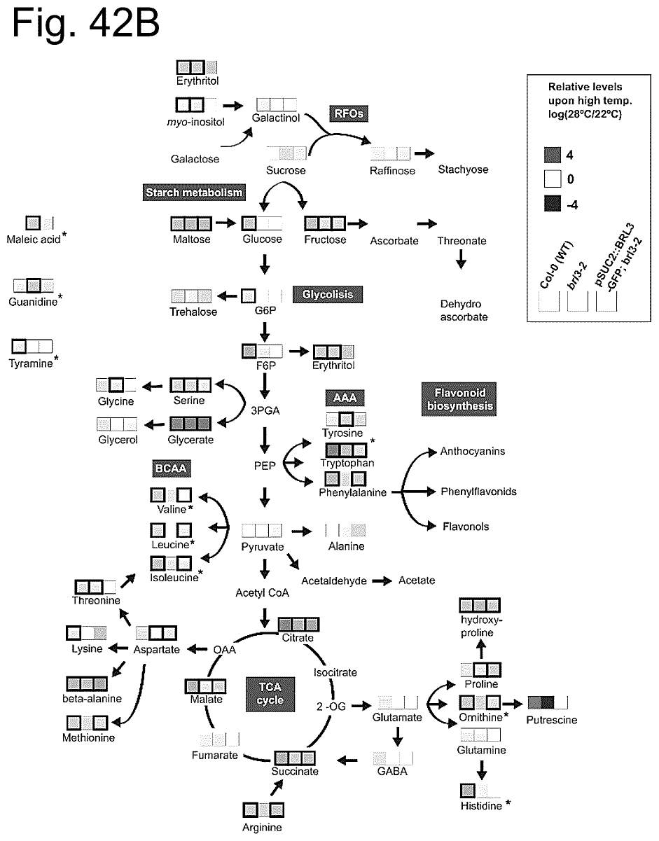

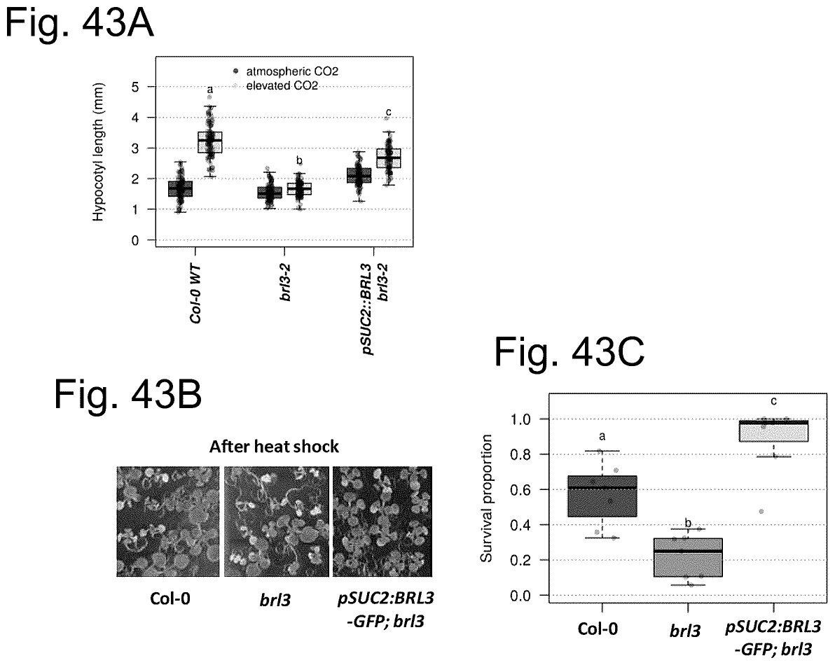

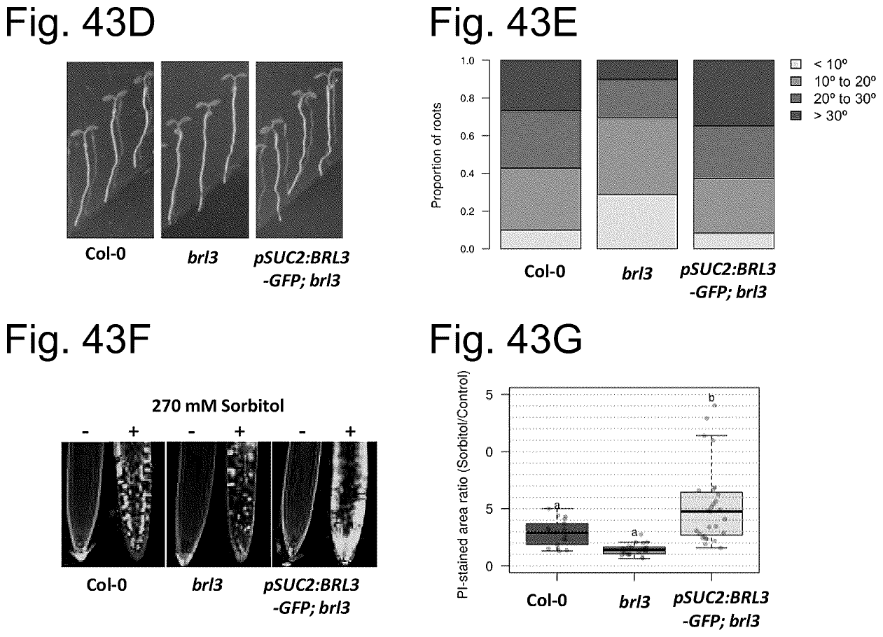

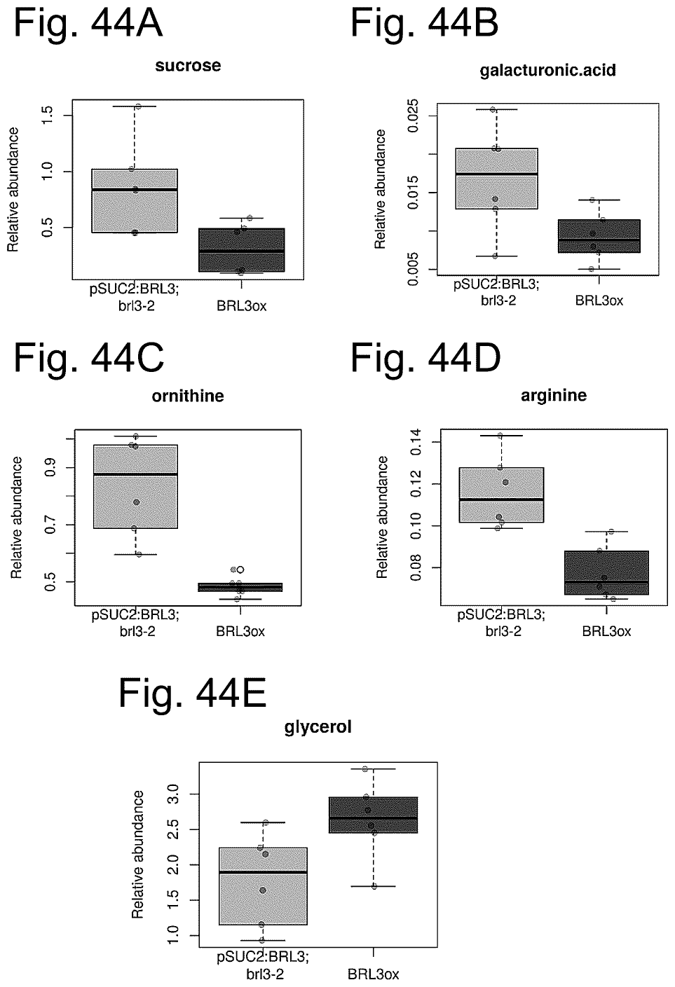

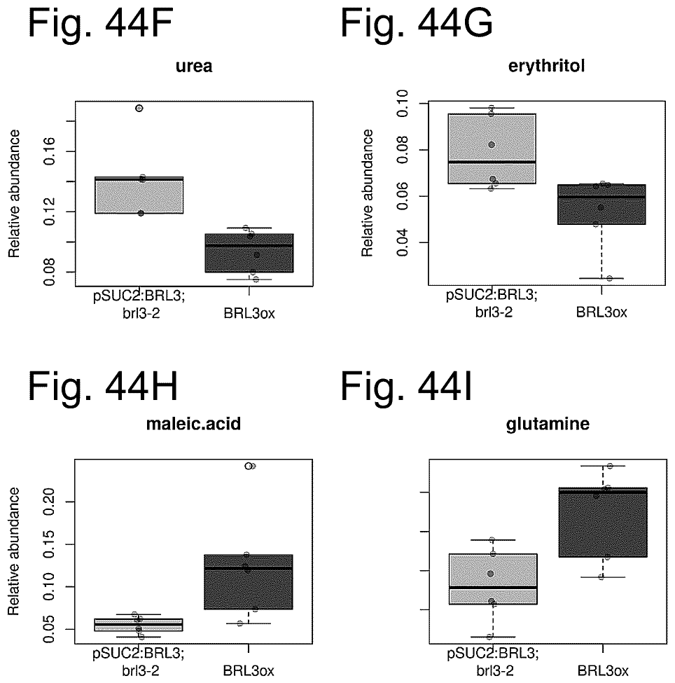

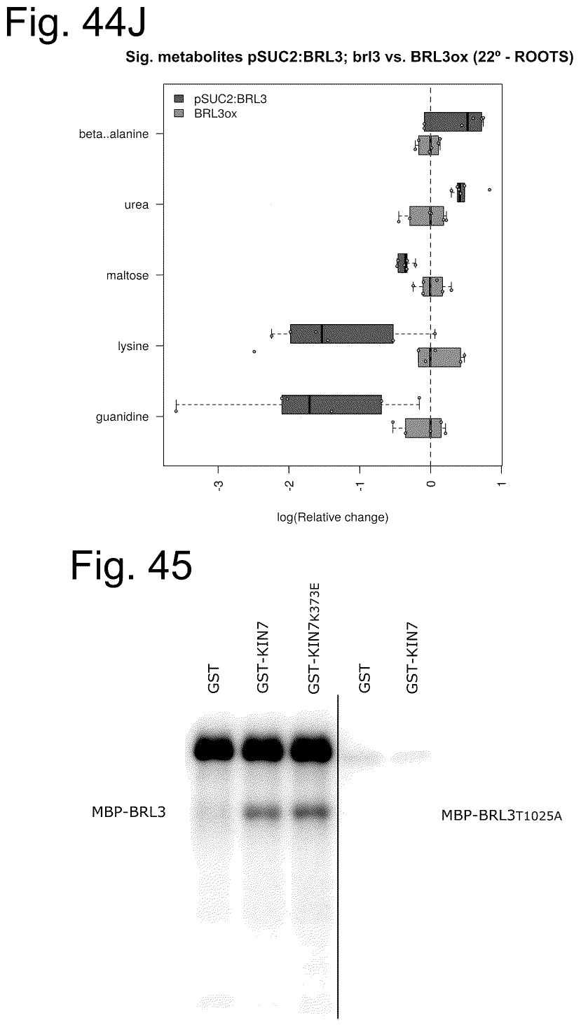

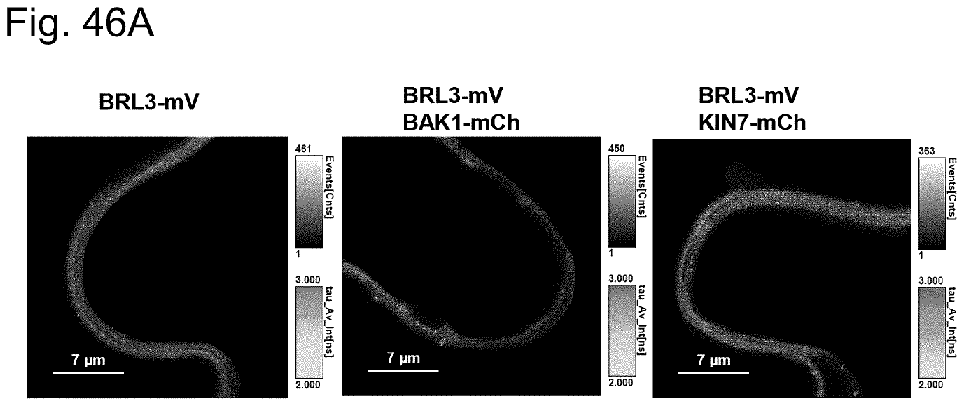

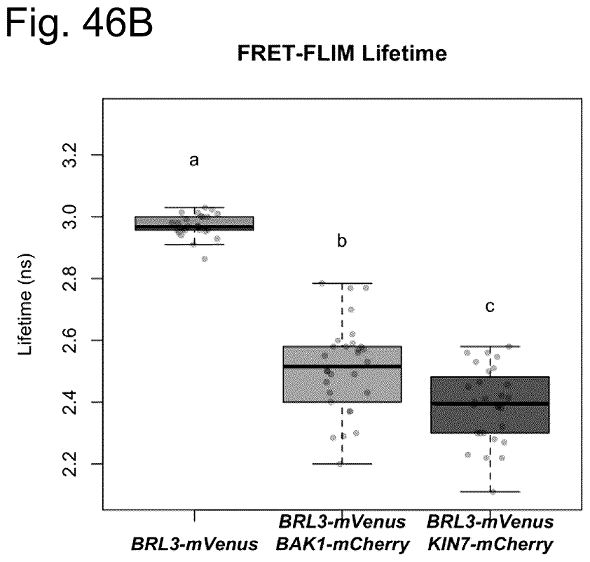

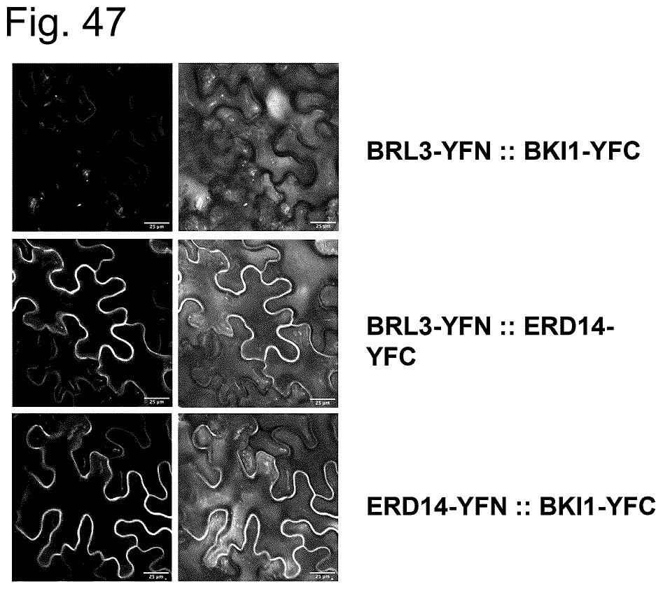

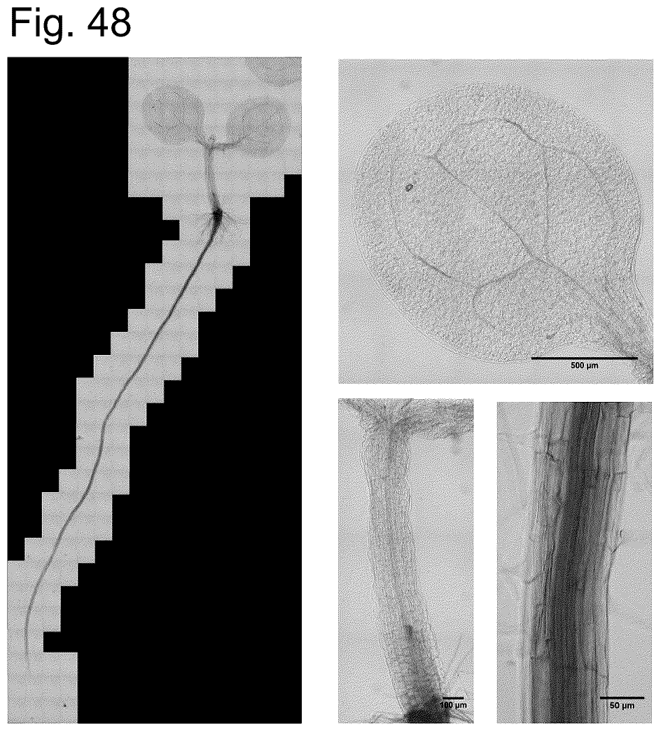

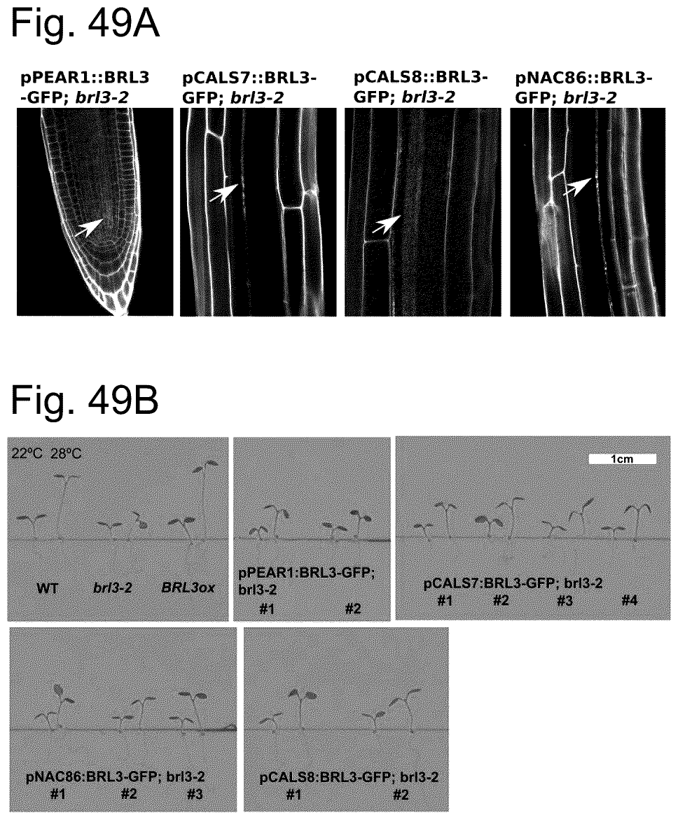

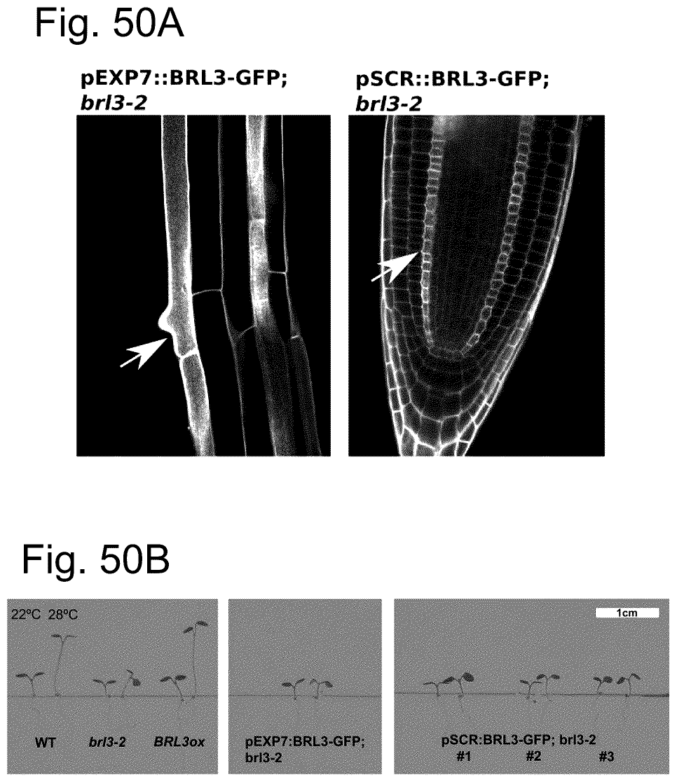

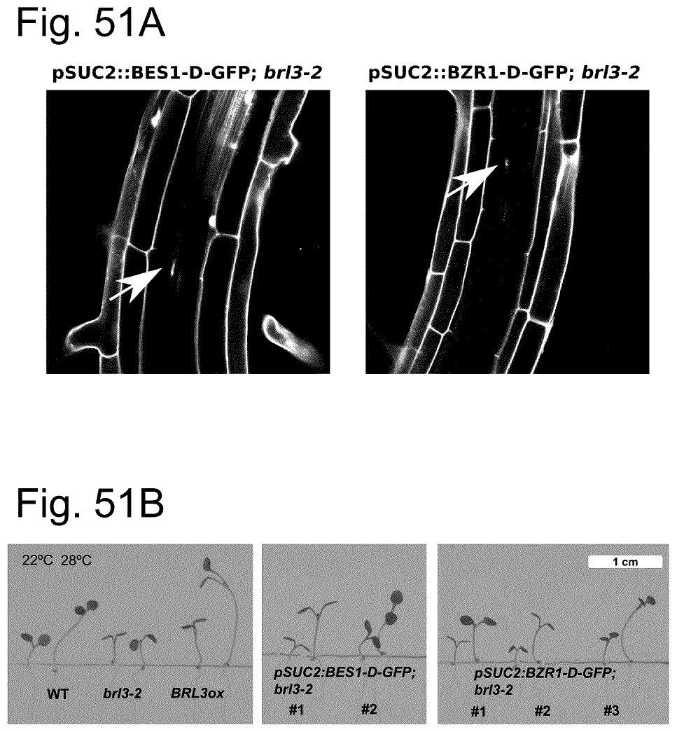

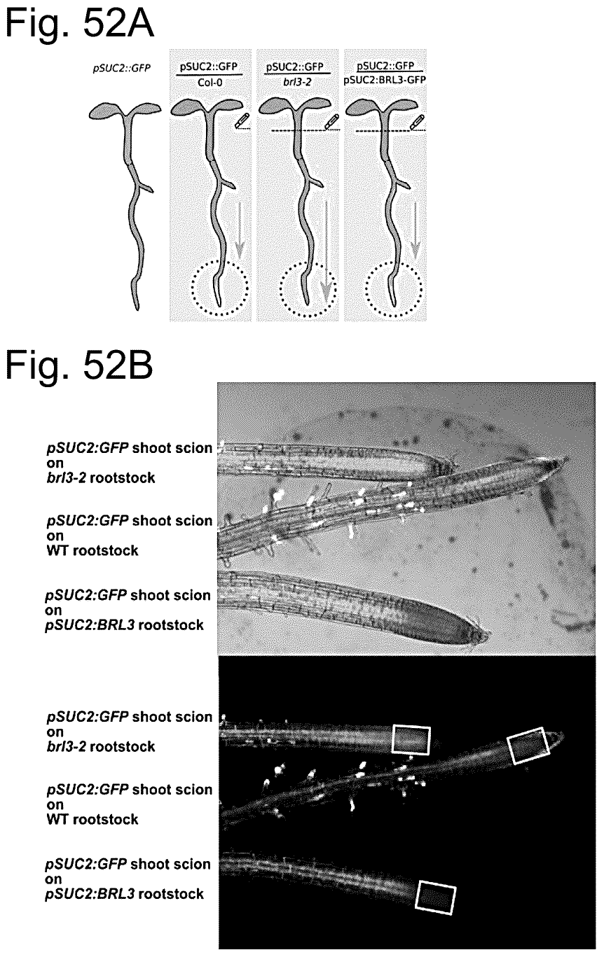

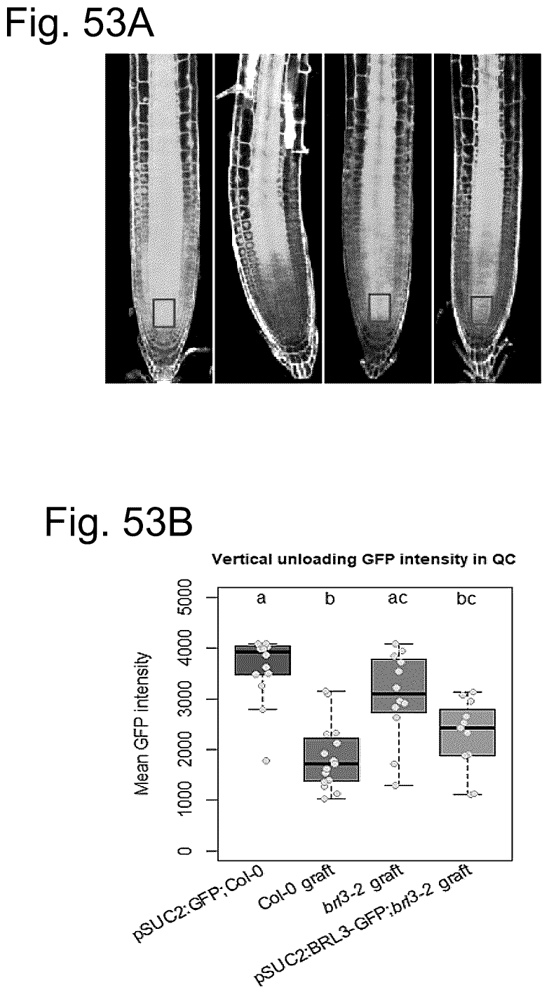

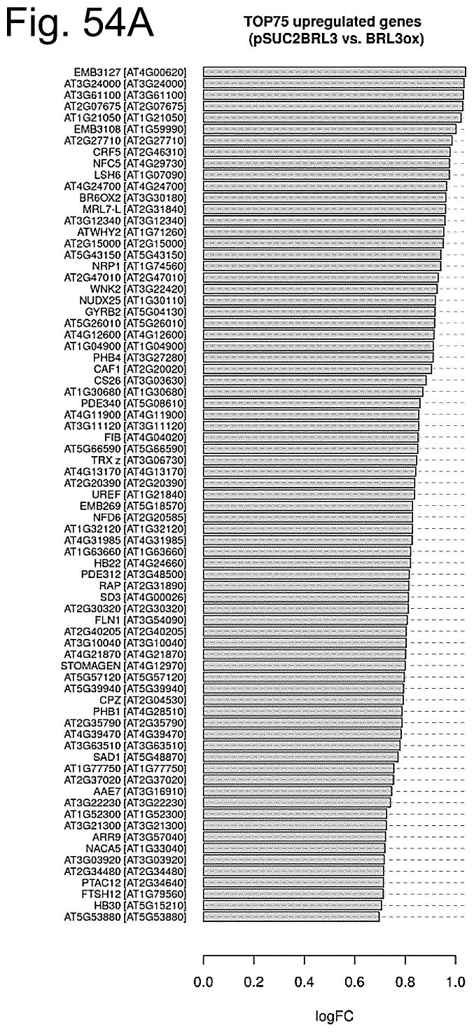

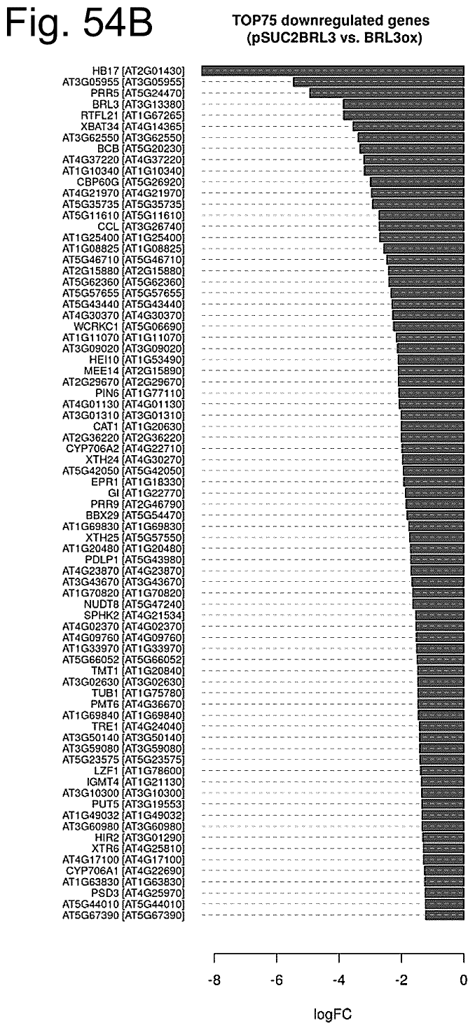

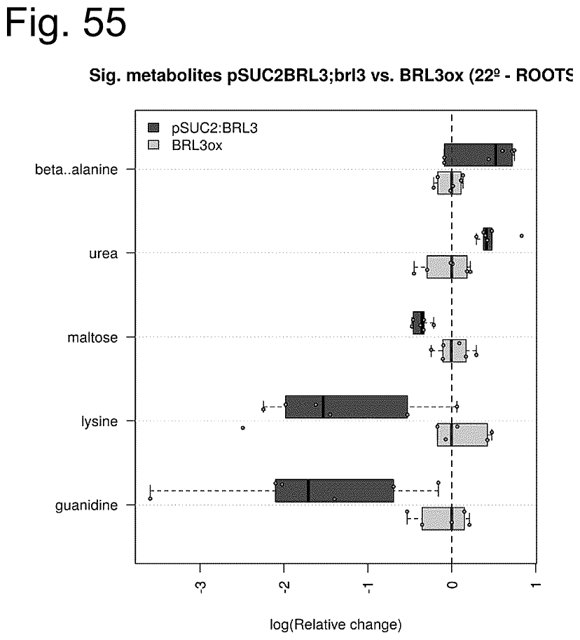

Root hydrotropism is an adaptive response of plants underwater limiting conditions representing a fast response of roots to direct their growth towards zones of water availability (Takahashi, et al., 2002). Whereas hydrotropic responses were impaired in the brl3-2 mutant, the specific expression of BRL3 in the phloem of the root was able to recover and enhance hydrotropic responses over the wild type levels (FIG. 7).

### Phloem Companion Cells Specific Overexpression of BRL3 Promotes Programmed Cell Death in Root Meristems Under Imposed Osmotic Stress

Another early response for dehydration or osmotic stress is programmed cell death (PCD) in the meristematic tissues exposed to the stress. The controlled death of these cells allows the plant to favor the root development of other roots not exposed, thus re-configuring root architecture to adapt growth under water-limited conditions (Duan, Y. et al., 2010). Microscopy analysis of root tips exposed to short-term strong osmotic stress (8-10h) revealed cell death features (staining with propidium iodine), that were reduced in the brl3-2 mutant compared to WT (FIG. 8). However, these lack of PCD was recovered and enhanced by the expression of BRL3 just in the phloem (pSUC2:BRL3-GFP; brl3-2, FIG. 8).

### BRL3 Expression in the Phloem Enhances Plant Adaptive Growth to Elevated Temperatures

High temperatures is a stress that greatly affects plant growth and development. Natural plant responses in Arabidopsis seedlings include the stimulation of hypocotyl growth when exposed to higher ambient temperature. This natural adaptation of young seedlings, known as thermomorphogenesis, contribute to better dissipate heat between the growing leaves and help the plant to cope with elevated temperatures (Casal and Balasubramanian, 2019). In wild type plants this response results in a significant increase of hypocotyl length when exposed to elevated ambient temperatures (28° C.), whereas the brl3-2 mutant seedlings are defective in this elongation. However, the pSUC2:BRL3-GFP; brl3-2 plants show greater hypocotyl elongation upon elevated ambient temperature (FIG. 9), revealing the capacity of BRL3 signaling uniquely in the phloem to drive and enhance the thermomorphogenesis process. Analogous results were obtained with other phloem-specific promoters (FIG. 49), namely PEAR1, CALS7, CALS8 and NAC86 (SEQ ID NO: 20, 21, 22, 23).

To check if this effect is specific of phloem companion cells (SUC2 promoter) and if it is expandable to other vascular tissues, we drove the expression of BRL3 (SEQ ID NO: 1) under the vascular-specific WOL (wooden leg) promoter (SEQ ID NO: 14). The WOL expression pattern involved the whole stele region, comprising phloem, xylem and procambium (Marques Bueno et al., 2016). The expression of BRL3 just under WOL promoter was able to enhance elevated temperature hypocotyl elongation to similar extends than the pSUC2:BRL3-GFP, brl3-2 line (two independent transgenic lines, FIG. 10).

Further, to test if the enhanced thermo-morphogenesis achieved with BRL3 expression just in vascular tissues is specific from the vasculature or the signals from other tissue/cell types have the same effect, we drove the BRL3 expression (SEQ ID NO: 1) just in the root quiescent center (QC)/stem cell niche (SCN) region via WOX5 promoter (SEQ ID NO 15) (Marques Bueno et al., 2016). In such case, it was not able to rescue the defects of brl3-2 mutant in hypocotyl elongation upon elevated temperature (two independent transgenic lines, FIG. 11). Defects in hypocotyl elongation under elevated temperatures found in the br13-2 mutants were neither rescued by the target expression of BRL3 in outer cell layers endodermis and epidermis (FIG. 50), driven by SCR and EXP7 promoters respectively (Marques-Bueno et al., 2016, SEQ ID NO: 24, 25). These results strengthen our hypothesis that plant stress adaptation via BRL3 pathway is occurring specifically from the phloem cells.

In order to re-evaluate adaptation to elevated temperatures from in plant adult stages, we assessed morphological changes upon exposure to higher ambient temperature in growth chambers. In Arabidopsis, elevated temperatures trigger the elongation of petioles cause the Arabidopsis rosettes to acquire a more open structure enabling better heat dissipation to cope with increased environmental temperatures (Casal and Balasubramanian, 2019). The brl3-2 mutants showed impaired petiole elongation upon elevated temperatures whereas the expression of BRL3 in the phloem, pSUC2:BRL3-GFP; brl3-2 line, was able to recover petiole elongation when exposed to warmer ambient temperature (FIG. 12).

Further, the brl3 mutants exhibit wild-type Ike growth in favorable temperature (22° C.) growth conditions (in agreement with (Caño-Delgado A. et al., 2004), yet a reduced hypocotyl (cell) size was observed when plants were exposed to elevated temperature (28° C.) (FIGS. 28A-D and 32). The conditional hypocotyl elongation defect of brl3 mutants (FIG. 28C-D) was, however, restored by expressing native pBRL3:BRL3-GFP construct in the brl3 mutant background (FIG. 33). Further phenotypes associated to thermo-morphogenesis such as petiole elongation and flowering time (Gray W. M. et al., 1998; Koini M. A. et al., 2009; Casal S. Balasubramanian J. J. et al., 2019) were also found to be perturbed in brl3 mature plants (FIGS. 28E-F and 34). By contrast, reduction of BRI1 signaling (bri1 mutants or WT plants treated with BR-synthesis inhibitor BRZ220) led to reduced hypocotyl size irrespective of the temperature (FIG. 35). These results unveil the existence of BR-mediated signaling cascade through BRL3 receptor to modulate growth under environmental stress.

In order to check if these early features of adaptive growth to elevated temperatures translated in an improved plant fitness, 21-day-old plants grown in normal conditions (22° C.) were subjected to a heat stress period. Temperature in the chambers were elevated to 37° C. for 10 consecutive days, which causes evident damage signs in the control plants, such as reduction in chlorophyll content and leaf necrosis (FIG. 13). pSUC2:BRL3-GFP; brl3-2 plants, however, retained a healthy appearance (FIG. 13). Then, temperatures were returned to normal conditions (22° C.) for 5 days more in order to check survival rates. From brl3-2 plants only the 83% survived, whereas the expression of BRL3 just in the phloem increased the survival rate until 100% (FIG. 13). These results show the relevance of phloem-specific brassinosteroid signaling in stress adaptation, particularly in heat stress, and also support the adaptive growth features previously described here, as early signs of better plant adaptation to elevated temperatures.

### BRL3 Signaling in the Phloem Utilizes Downstream Component of the Brassinosteroid Response Pathway to Regulate Plant Adaptation to High Temperatures.

The analysis of deregulated genes due to BRL3 expression in the phloem revealed a significant enrichment in genes previously identified as direct targets of the canonical transcription factors downstream the brassinosteroid signaling pathway, BES1 and BZR1 (SEQ ID NO 7, 9) (Sun et al., 2010). These targets appeared in deregulated genes much more than expected by a random selection (FIG. 14A). Common targets of BES1 and BZR1 being deregulated due to phloem BRL3 expression are disclosed in FIG. 14B. To further address the utilization of canonical brassinosteroid signaling pathway we endogenously applied Bikinin, a known inhibitor of BR signaling negative regulator BIN2 (SEQ ID NO 11) (De Rybel et al., 2009). Bikinin was able to promote hypocotyl elongation in WT and brl3-2 mutant in both control and high temperature conditions (FIG. 14 C, D). Further, the elevated temperature regulated BES1 dephosphorylation was also impaired in brl3-2loss of function mutant (FIG. 14C). These results conclude that the activation of canonical BR pathway components downstream BRL3 is needed for stress response, and thus, that the observed effects in phloem overexpression of BRL3 are also through these components. Genes deregulated due to BRL3 expression in the phloem and being described targets of both, BES1 and BZR1 are disclosed in FIG. 15.

The importance of downstream BRL3 signaling by BES1 and BZR1 was further shown in brl3 mutants, where the defective hypocotyl growth of brl3 mutants was rescued by a gain-of-function bes1-d mutation (FIG. 28G-H). Strikingly, brl3 mutants also displayed a reduced BES1 dephosphorylation pattern (FIG. 281). Further, exogenous application of bikinin rescued brl3 conditional phenotypes on elevated temperature (FIG. 36) supporting the hypothesis that BRL3 uses downstream cascade of the BRI1−pathway to promote adaptive growth upon elevated temperatures.

Finally, the targeted expression of constitutively-active forms of the transcription factors BES1 and BZR1, the variants BES1-D and BZR1-D (Yin et al., 2002; Wang et al., 2002, SEQ ID NO 27, 28, 29, 30), in the phloem companion cells driven by SUC2 promoter (SEQ ID NO 13) were able to recapitulate the hypocotyl growth defects under elevated temperatures observed in the brl3-2 mutants (FIG. 51). Altogether, these results confirms the participation of these two transcription factors in the signaling pathway initiated by BRL3 from the phloem.

Further, the master regulator of brassinosteroid-signaling BES1 and BZR1 are known to work alongside the light/temperature-regulated transcription factor PIF4 and the auxin-response factor ARF6 to cooperatively regulate large numbers of common target genes. This forms a molecular circuit that that integrates hormonal, signals to regulate elongation growth in response to different environmental signals such as thermomorphogenesis upon high temperature in Arabidopsis (Oh et al., 2014; Quint et al., 2016; Ibanez C. et al, 2018; Martinez C. et al, 2018). We analysed if among pSUC2:BRL3-GFP; brl3-2 deregulated genes there was an over-representation of direct targets of well-known thermomorphogenesis regulators, PIF4 and ARF6. We found that these targets appeared in deregulated genes much more than expected by a random selection (FIG. 16A). The great overlap observed between BES1, BZR1, PIF4 and ARF6 targets genes being deregulated in pSUC2:BRL3-GFP; brl3-2 (FIG. 16B), suggest that phloem-specific BRL3 signaling trigger responses that overlaps with the responses controlled by main thermomorphogenesis regulators.

### BRL3 Signaling in the Phloem Drives Major Transcriptional Changes in Biotic and Abiotic Stress Responses and Metabolic Rearrangements.

Furthermore, among the deregulated genes due to phloem BRL3 expression, we found a massive deregulation of genes involved in abiotic stress responses. These genes are classified in subcategories involving specific stresses and deployed in FIG. 17.

Additionally, we found that just the expression of BRL3 in the phloem is able to drive the changes in metabolite levels in control conditions, which importantly involve the accumulation of amino acids alanine and the osmoprotectant proline (Szabados and Savoure, 2010), whereas provoking the decrease of glutamine and glutamic acid, the sugar maltose and carboxylic acids (FIG. 18A). Metabolic analysis of plants grown under elevated temperatures (28° C.) revealed the accumulation of alanine and glutamine in pSUC2:BRL3-GFP; brl3-2 plants and the decrease of glycerol and glutamic and pyruvic acids (FIG. 18B).

Likewise, when compared metabolite levels between pSUC2:BRL3-GFP and BRL3ox plant shoots, we found 9 differentially accumulated metabolites (FIG. 44). These included relevant metabolites for stress response and nutrient efficiency. Sucrose, the main output of photosynthesis and main energy source molecule transported from shoot to roots was accumulated in pSUC2:BRL3-GFP plants respect the overexpression. Sucrose is accumulated during stress periods and increased levels are related with better root:shoot ratio grow, a key trait to withstand drought stress (Durand et al., 2016, Chen et al., 2022). In addition, levels of metabolites glutamine, omithine, arginine and urea suggest an increased organic nitrogen storage (in form of arginine) in pSUC2:BRL3-GFP plants. The decreased levels of glutamine matches with an increased metabolic flow towards arginine biosynthesis (increased levels of arginine and the intermediate omithine), also resulting in higher levels of urea that is a product of arginine degradation and that can serve as an alternative nitrogen source (Winter et al., 2015). Arginine is also the precursor for synthesis of polyamines (e.g. putrescine), compounds demonstrated to provide stress tolerance (Winter et al., 2015). We also detected accumulation of beta-alanine in the roots of pSUC2:BRL3-GFP plants (FIG. 44J), a metabolite that act as defence compound against stresses, including both, biotic and abiotic stress (Parthasaranthy et al., 2019).

Besides abiotic stress, transcriptomic hallmarks of phloem expression of BRL3 (pSUC2:BRL3-GFP: brl3-2) revealed biotic stress responses (plant immune response) as the most affected category among the deregulated genes. The massive deregulation of genes annotated in this category, support that phloem expression of BRL3 also affects biotic stress response mechanisms. Deregulated biotic stress response genes are deployed and classified in specific responses in FIG. 19. Further transcriptome analysis of brl3 mutants uncovered that, at ambient temperature (22° C.), BRL3 activated genes were enriched in growth, development, homeostasis, light response and tyrosine metabolism GO categories, and BRL3-repressed genes were enriched in immune response, redox status and jasmonic acid-mediated response GO categories (FC>1.5, FDR<0.05; FIG. 37). The comparison of transcriptomic profiles of M3 at ambient and elevated (28° C.) temperature (interaction term, see methods, FIG. 38A) revealed that genes involved in redox and abiotic stress responses (such as heat and water deprivation) failed to be activated the brl3 mutant (under-response, FIG. 29A. right). By contrast, negative regulators of ethylene and carbon catabolic processes responded stronger in the brl3 mutant than in WT (over-response, FIG. 29A left). Among genes annotated in response to abiotic stress (FIG. 29B), we found genes known to promote thermotolerance such as TEMPERATURE-INDUCED LIPOCALIN (TIL), HEAT SHOCK TRANSCRIPTION FACTOR A7A (HSFA7A), and MULTIPROTEIN BRIDGING FACTOR 1C (MBF1C), (Chi W. T. et al, 2009; Larkindale J. et al, 2008; Suzuki N. et al, 2005), and genes that promote osmoprotectant accumulation in response to temperature, such the GALACTINOL SYNTHASE1 (GolS1), (Panikulangara T. J. et al., 2004) in agreement with previous report on BRL3 overexpression lines (BRL3ox) (Fabregas N. et al., 2018). Further, core thermo-morphogenesis genes such as those annotated as direct targets of BES1/BZR1, PIF4, AUXIN RESPONSE FACTOR 6 (ARF6) and HOMOLOG OF BEE2 INTERACTING WITH IBH 1 (HBI1) were also found to be significantly enriched (FIG. 38B; (Sun Y. et al, 2010; Yu X. et al., 2011; Oh E. et al, 2014; Oh E. et al, 2012; Bai M. Y. et al, 2012). These results suggest that BRL3 is necessary for the normal activation of high temperature stress response genes.

Indeed, the bd3 mutant seedlings were more sensitive to extreme temperatures (reduced survival) when subjected to a heat-shock treatment (150 min at 42° C.) (FIGS. 29C-D) and showed impaired response to osmotic stress in terms of hydrotropism (root growth reorientation towards water availability) (Takahashi N. et al, 2002), and cell death in the root apex (Duan Y. et al., 2010) (FIG. 29E-1). Conversely, BRL3ox were hyper-responsive for both these traits (Fabregas N., et al, 2018) (FIG. 29E-1). When taken together, these data suggest that the BRL3 pathway is central for plant physiological and molecular adaption to climate stress.

In order to elucidate phloem BRL3-driven transcriptional changes that contribute to temperature adaptation, we compared the transcriptomes of the pSUC2:BRL3-GFP; 3 line and brl3 mutant shoots at ambient and elevated temperatures (FIGS. 31A and 40) Genes with stronger response to temperature due to phloem specific-BRL3 expression were found to be enriched in energy generation processes (over-response, FIG. 31A left). These genes included genes associated with photorespiration as well as NADH-ubiquinone oxidoreductases and ATP syntheses inked to mitochondrial electron transfer chain, which in turn have great impact on redox homeostasis (Rhoads D. M. et al., 2008). By contrast, genes involved in auxin transport, (negative regulators of) ethylene signaling and steroid signaling, the later probably due to feedback mechanisms, displayed less deregulation in the pSUC2:BRL3; brl3 lines than in the M3 mutant subjected to increased temperatures (under-response, FIG. 31A right) suggesting partial rescue by BRL3 in the phloem. The mild repression of the photorespiration machinery observed in pSUC2:BRL3; brl3 might result in better carbon utilization at elevated temperatures, as CO2/O2 selectivity of RuBisCO decreases under such conditions thereby inducing photorespiration pathway (Cui L. L. et al., 2016; Cavanagh A. P. et al., 2021). Collectively, these results show significance of BRL3 signals from inner phloem companion cells in balancing plant adaptive growth by modulating plant energy status and hormone responses.

Given that BRL3 receptors are natively expressed in phloem vascular tissues throughout the plant (Caño-Delgado A. et al., 2004; Fabregas N. et al., 2013) (FIG. 398A-G), we sought to investigate the cell-specific contribution of BRL3 signals to the observed phenotypes, by expressing BRL3 under different cell-type-specific promoters in the brl3 mutant background (Marqués-Bueno M. M. et al., 2016) (FIGS. 30A-G and 39H-1). Driving BRL3 expression generally in the vasculature (pWOL:BRL3-GFP; brl3) or uniquely to the phloem companion cells (pSUC2:BRL3-GFP; brl3) rescued the thermomorphogenesis defects of brl3 mutants (FIGS. 30H-I and 39J-K). Further, pBRL3:BRL3-GUS reporter analysis revealed that BRL3 is spatially regulated upon elevated temperature i.e. induced in hypocotyl vasculature and repressed in the root vascular tissues (FIG. 30J). By contrast, BRL3 expression in either the epidermis (pGL2-BRL3-GFP; brl3), root SCN (pWDX5:BRL3-GFP; brl3) or the meristem (pRPS5A:BRL3-GFP; brl3) failed to rescue the thermo-morphogenic defects of the brl3 mutants (FIG. 30H-1). The epidermis has been proposed as a major site of action for generally controlling plant growth (Savaldi-Goldstein S. et al., 2007; Procko C et al., 2016; Kim S. et al., 2020). It seems plausible that vascular cells of the phloem have the ability to modulate growth adaption via BRL3, independently of BRI1 (FIGS. 35A-B S4A and B). Collectively, our data thus demonstrate that BRL3 receptor signaling modulates plant adaptive growth to climate stress from the inner vascular cells of the phloem.

### Phloem-Specific BRL3 Expression Reveals Exclusive Omics Features Respect BRL3 Overexpression (BRL3ox)

We compared the transcriptome of the phloem-specific BRL3 expression (pSUC2:BRL3-GFP) against BRL3 overexpression (BRL3ox, non-exclusive phloem expression) in order to find distinctive hallmarks of BRL3 expression in the phloem. We found a total of 275 transcripts differentially regulated, being 130 and 145 genes up and down regulated respectively. Top 150 deregulated genes are shown in FIG. 54. Among downregulated genes, we detected an over-representation of genes involved into oxidative stress responses (GO enrichment analysis).

### Mutants for Non-Canonical Components Contributing to BRL3 Signaling are Also Affected in Adaptive Growth (Thermomorphogenesis)

In order to uncover new components that are affecting the phloem-specific signaling through BRL3, we looked forward to leverage our transcriptomic data in combination with proteomics data (Fàbregas et al. 2013) through computational pipelines. Protein-protein modelling of identified components that are in complex with BRL3 at the membrane, allowed us to estimate and rank interaction affinities (Table 1, FIG. 20A). Interestingly we found a small LRR-RLK protein showing better affinity for BRL3 than the described canonical co-receptor BAK1 (Fábregas 2013). This receptor, which was uniquely identified in the BRL3 complex and not in complex with the other brassinosteroid receptors (FIG. 20B), is known as KIN7 (SEQ ID NO 3, 4) and has been involved in abiotic stress responses (Vialaret et al., 2014, Isner et al., 2018, Grison et al., 2019). Further, the KIN7 receptor was able to interact with BRL3 in tobacco leaves (FIG. 21A) and showed a phloem-specific expression pattern along the seeding (FIG. 21B), which support its contribution to phloem-specific BRL3 signaling. Further, the direct protein-protein interaction was also confirmed by FRET-FLIM (FIG. 46) and demonstrated to be functional on in vitro phosphorylation assays (FIG. 45). BRL3 was able to phosphorylate KIN7 and not the other way around. In addition, a phospho-dead version of BRL3 kinase, BRL3T102 (Modelled based on reported BRI1 kinase dead version in Wang et al., 2008), is unable to phosphorylate the kinase domain of KIN7, revealing that the kinase activity of BRL3 is required to phosphorylate KIN7 (FIG. 45). Altogether, these data support that BRL3 and KIN7 work together in signal transduction (signalling pathway) from the phloem. When the mutants for KIN7 (kin7-1, and its genetic cross with brl3-2) were analysed in terms of hypocotyl growth under elevated temperature, these mutants show the same phenotypic defects than the brl3-2 lines, supporting its participation in the BRL3 signaling during adaptive growth (FIG. 22).

Analogously, other protein identified exclusively in complex with BRL3 at the membrane, the Early-Response to Desiccation ERD14 (Fábregas et al., 2013, SEQ ID NO 5, 6), interacted with BRL3 in tobacco plants (FIG. 47) and showed a prominant vascular expression pattern (FIG. 48). Mutants for ERD14 were unable to elongate hypocotyls under elevated temperatures, supporting that BRL3 and ERD14 function together transducing signals for adaptative growth (FIG. 23)

In alternative approach we used transcriptomic data of BRL3 overexpressor plants exposed to drought (Fàbregas et al., 2018) and spatio-temporal transcriptomic maps of Arabidopsis roots (Brady et al., 2007) in order to identify putative new components downstream of phloem BRL3 signaling. These putative new components were selected according a combination of factors as (i) very strong deregulation by BRL3, (ii) differential response to drought conditions in BRL3ox plants respect Col-0 and (iii) predicted preferential expression in phloem and vascular root tissues. A total of 11 transcription factors were selected (Table 2; SEQ ID NO 31-52). Interestingly 10 out 11 mutants for the selected transcription factors showed thermomorphogenesis defects (FIG. 24), similarly to brl3-2 mutant. This results further support the effect of phloem-specific BRL3 signaling in adaptive growth to elevated temperatures and involves new non-canonical transcription factors downstream BRL3.

### Phloem-Specific Expression of BRL3 Promotes Root-Ward Transport

The main role of phloem tissues in a plant is to transport products of photosynthesis from source tissues (leaves) to sink tissues (roots). To observe if the transport of photosynthates in the phloem was affected we tested whether local application of sucrose, was sufficient to impact root growth. Exogenous sugar application is known to promote primary root elongation as well as lateral root emergence in plants (Kircher and Schopfer, 2012; Gupta et al., 2015), so we use it as a readout for phloem transport. We found that plants lacking functional BRL3 (brl3-2 mutant) showed impaired root responses, whereas plants expressing BRL3 uniquely in phloem (pSUC2:BRL3-GFP; brl3-2) showed enhanced root responses due local application of sucrose in shoot tissues (FIG. 25). These results support that phloem-specific expression of BRL3 increases the transport capability of the plant.

Metabolic analysis also supports an increase in the transport capability of pSUC2:BRL3-GFP; brl3-2 plants, as metabolites whose global levels do not change between pSUC2:BRL3-GFP; brl3-2 and brl3-2 mutant but that show differences in tissue distribution are always accumulated in pSUC2:BRL3-GFP; brl3-2 roots (FIG. 26). Importantly, among these metabolites we found the osmoprotectant sugar myo-inositol (Sengupta et al., 2012).

Further, many genes coding transporter domains were found to be deregulated due to the expression of BRL3 uniquely in phloem cells (FIG. 27). We selected transcripts whose protein sequences were annotated at least with one of the following InterPro domains: amino acid transporter, oligopeptide transporter, transporter from major facilitator superfamily (MFS), sugar transporter or ABC-transporter to define this category manually.

### Phloem-Specific Expression of BRL3 Restricts Phloem Unloading in Roots

Since BRL3 from phloem companion cells regulates adaptive growth, we investigated whether BRL3 can affect phloem function. We used pSUC2:GFP reporter lines to check symplastic unloading of the free GFP signals in the roots (Imlau A. et al., 1999; Stadler R. et al., 2005). In plants obtained by the micrografting of pSUC2:GFP shoot scions onto brl3 rootstocks displayed a significantly higher GFP signal at the root tip as compared to plants comprising of pSUC2:GFP shoot scions and a WT rootstock (FIG. 41) suggesting enhanced phloem unloading in the roots of brl3 mutant. These observations point towards a potential role for BRL3 in nutrient and energy partitioning required for adaptive growth. To test this hypothesis, we performed metabolic profiling of WT, brl3 and pSUC2:BRL3-GFP; brl3 seedlings grown under ambient or elevated temperatures. The metabolic responses to elevated temperature were very conserved between WT and the brl3 mutant shoots and roots, (FIG. 42). However, when comparing the metabolic footprint of shoot tissues of bd3 (vs. WT) and pSUC2:BRL3-GFP; bd3 (vs. bd3) at ambient temperature, tri-carboxylic acid (TCA) cycle intermediates, as well as the intimately associated metabolites glutamine, glutamic acid, alanine and pyruvic acid (FIG. 31B) showed significantly altered levels in both comparisons, indicating that they were controlled by BRL3 action within the phloem. Strikingly, most of the changes were in the same direction (decreased) with the exception of pyruvic acid which changes in an opposite direction (accumulated in brl3 and decreased in pSUC2:BRL3-GFP; brl3 shoots) (FIG. 31B). Pyruvic acid is a key central metabolite involved in several pathways of primary metabolism including glucose/gluconeogenesis and as an entry point into the TCA cycle. The accumulation of pyruvic acid in the mutant might indicate a poor carbon utilization (e.g. faster degradation of starch or stunted photosynthesis) and a slower TCA cycle resulting in a lower accumulation of intermediates (succinate, fumarate), reducing power and H+ gradient. On the other hand, decreased levels of pyruvate in pSUC2:BRL3-GFP; brl3 plants could indicate a faster incorporation into TCA and increased flux, which would also result in a lower accumulation of intermediates (FIG. 31C-G) but an increased H+gradient and reducing power for mitochondrial respiration (Shen W. et al., 2006). The combined evidence suggests that BRL3 signaling from vascular phloem cells seems to boost central carbon metabolism, impacting the plant energy status and offering extra energy supply under challenging conditions. In addition, amino acid proline which functions as an osmoprotectant also accumulated in plants expressing BRL3 solely in the phloem (FIG. 31H).

Since BRL3 could potentially affect both photorespiration and carbon metabolism, we analyzed if BRL3 could modulate plant growth under elevated CO2 levels, a key climate factor contributing to global warming. In Arabidopsis, short-term exposure to high CO2 levels promotes the net rate of photosynthesis and hence stimulate overall plant growth (Van Der Kooij T. A. et al., 2012; Robinson E. A. et al., 2012). The brl3 mutant was defective for hypocotyl elongation growth, whereas both the pSUC2:BRL3-GFP; brl3 line and BRL3ox seedlings showed enhanced hypocotyl growth upon elevated CO2 (FIGS. 31I, and 43A). Phloem-companion cel specific expression of BRL3 (pSUC2:BRL3-GFP; brl3) also rescued the defects in other climate stress responses such as heat and reduced water availability (FIGS. 31J and 43B-G) indicating BRL3 signals from the phloem companion cells are key for optimal plant growth and survival during stressful conditions.

We next used pSUC2:GFP reporter lines to check the movement and the symplastic unloading of the free GFP (Stadler et al., 2005) as a proxy to evaluate the phloem unloading capabilities in the roots. We generated chimeric plants by micrografting (Melnyk, 2016) pSUC2:GFP shoot scions onto WT, brl3 mutants or phloem-expressing BRL3 (pSUC2:BRL3-GFP) rootstocks (FIG. 52A). The brl3 mutant rootstocks plants displayed a significantly higher GFP signal at the root tip as compared to plants comprising of pSUC2:GFP shoot scions and a WT rootstock (FIG. 52B, 53), whereas grafts of pSUC2::GFP shoot scions on pSUC2:BRL3 rootstocks showed a restricted diffusion of free GFP (FIG. 52B, 53). These results support that BRL3 signaling in the phloem restrict phloem unloading in the roots, suggesting a potential role for BRL3 in nutrient and energy partitioning required for adaptative growth (Ham and Lucas, 2014).

### REFERENCES

Aljanabi S. M. et al. (1997) Nucl. Ac. Res., 25(22):4692-4693.
Bai M. Y. et al (2012) Nat Cell Biol. 14(8):810-7. doi: 10.1038/ncb2546.
Barajas-Lopez J. D. et al. (2021) Plant Cell Phyisol. 62(1):80-91. doi: 10.1093/pcp/pcaa139.
Bartusch K. et al. (2020) Front Plant Sci. 10;11;613442. doi: 10.3389/fpls.2020.613442.
Betegón-Putze I. et al. (2019) Plant J. 98(6):1145-1156.
Blum M. et al. (2020) Nucleic Acids Res. 49(D1):D344-D354. doi: 10.1093/nar/gkaa977.
Boyer J. S. et al., (1982) Science 218: 443-448.
Brady S. M. et al., (2007) Science 318: 801-806
Caño-Delgado A. et al., (2004) Development 131, 5341-5351.
Caño-Delgado A., et al., (2009) Methods Mol Biol. 495:81-8.
Casal J. J. and Balasubramanian S (2019) Ann Rev Plant Biol. 70:1, 321-346.
Cavanagh A. P. et al (2021) Plant Biotechnol J. 20(4):711-721. doi: 10.1111/pbi.13750.
Chen Q. et al., (2022) Nat Plants. 8(1):68-77. doi:10.1038/s41477-021-01040-7
Choe S. et al., (2001) Plant J. June; 26(6):573-82.
Chi W. T. et al (2009) Plant Cell Environ. 32(7):917-27. doi: 10.1111j.1365-3040.2009.01972.
Chinchilla D, et al (2007) Nature. July 26; 448(7152):497-500. doi: 10.1038/nature05999. Epub 2007 Jul. 11. PMID: 17625569.
Clough S. J., Bent A. F. (1998) Plant J. 16(6):735-43.
Cui L. L. et al (2016) Front Plant Sci. 7:1165. doi: 10.3389/fpis.2016.01165.
De Rybel B. et al. (2009) Chem Biol. 16(6):594-04.
Duan, Y. et al. (2010) New Phytol. 186, 681-695.
Durand M. et al., (2016) Plant Physiol. 170(3):1460-1479. doi:10.1104/pp. 15.01926
Fàbregas N. et al., (2013) Plant Cell 25:3377-3388.
Fàbregas N. et al., (2018) Nature Communications. 9:4680
Friedrichsen et al., (2000) Plant Physiol. August; 123(4):1247-56.
Furuta K M. et al., (2014) Science. 345(6199):933-937. doi:10.1126/science.1253736.
Gampala, S. S., et al. (2007). Dev. Cell 13:177-189.
Gray W. M. et al. (1998) Proc Natl Acad Sci USA. 95(12):7197-202. doi: 10.1073/pnas.95.12.7197.
Grison, M. et al., (2019) Plant Physiol. 181(1), 142-160
Guo Q, et al (2018). Proc Natl Acad Sci USA. November 6; 115(45):E10768-E10777. doi: 10.1073/pnas.1811828115. Epub 2018 Oct. 22. PMID: 30348775; PMCID: PMC6233084.
Gupta A. et al. (2015) Plant Physiol. 168(1):307-20.
Ham B K, Lucas W K (2014). J Exp Bot. 65(7):1799-1816. doi:10.1093/jxb/ert417
Hentrich M, et al. Plant J. 2013 May; 74(4):626-37. doi: 10.1111/tpj.12152. Epub 2013 Mar. 25. PMID: 23425284; PMCID: PMC3654092.
Ibanez C. et al (2018) Curr Biol. 28(2):303-310.e3. doi: 10.1016/j.cub.2017.11.077.
Ikeda M, et al (2011) Plant Physiol. November; 157(3):1243-54. doi: 10.1104/pp. 111.179036. Epub 2011 Sep. 9. PMID: 21908690; PMCID: PMC3252156.
Imlau A. et al (1999) Plant Cell. 11(3):309-22. doi: 10.1105/tpc.11.3.309.
Isner, J. C. et al., (2018). Current Biology. 28(3), 466-472
Jenness, M. K. et al., (2019). Front Plant Sci. 10:806. doi: 10.3389/fpls.2019.00806.
Kangasjärvi S. et al (2012) J Exp Bot 64(3)1619-1636. https://doi.org/10.1093/jxb/err402
Kim S. et al (2020) Nat Commun. 11(1):1053. doi: 10.1038/s41467-020-14905-w.
Kinoshita et al., (2005) Nature. 2005 Jan. 13; 433(7022):167-71.
Kircher S., Schopfer P. (2012) PNAS. 109 (28) 11217-11221.
Koini M. A. et al. (2009) Curr Biol. 19(5):408-13. doi: 10.1016/j.cub.2009.01.046.
Kopka, J. et al. (2005) Bioinformatics. 21, 1635-1638.
Krishnamurthy, P. et al. (2020) Plant Physiol. 184(4):2199-2215. doi: 10.1104/pp. 20.01054.
Larkindale J. et al (2008) Plant Physiol. 146(2):748-61. doi: 10.1104/pp. 107.112060.
Lee K, et al, PLoS One. 2017 Jul. 26; 12(7):e0181804. doi: 10.1371/journal.pone.0181804. PMID: 28746399; PMCID: PMC5529009.
Li, J., Nam, K. H. (2002). Science 295: 1299-1301.
Lisec, J. et al. (2006) Nat. Protoc. 1, 387-396.
Luedemann, A., et al (2008) Bioinformatics. 24, 732-737.
Ma S. et al (2019) J Plant Growth Regul. 38:494-500. https://doi.org/10.1007/s00344-018-9864-1.
Martinez C. et al (2018) EMBO J. 7(23):e99552. doi: 10.15252/embj.201899552.
Marquès-Bueno M. D. M. et al. (2016) Plant J. 85(2):320-333.
Megan G Sawchuk et al (2013) Plant Signaling & Behavior, 8:11, DOI: 10.4161/psb.27205Mroue S. et al. (2018) J Exp Bot. 69(2):201-212.
Melnyk CW (2016) Regeneration (Oxford). 4(1):3-14. doi:10.1002/reg2.71.
Miyashima S. et al., (2019) Nature. 565(7740):490-494. doi: 10.1038/s41586-018-0839-y.
Muhammad A. et al. (2018) Plant metabolites and regulation under environmental stress, Academic Press, Chapter 8:153-167. https://doi.org/10.1016/B978-0-12-812689-9.00008-X.
Nolan et al., (2020) Plant cell DOI: https//doi.org/10.1105/tpc.19.00335.
Parthasarathy A, Savka M A, Hudson A O (2019) Front Plan Sci. 10:921. doi:10.3389/fpls.2019.00921.
Plans-Riverola et al., 2019 Development doi: 10.1242/dev.151894
Oh E. et al. (2014) eLife. 3: e03031.
Oh E. et al (2012) Nat Cell Biol. 14(8):802-9. doi: 10.1038/ncb2545.
Panikulangara T. J. et al (2004) Plant Physiol. 136(2):3148-3158. doi: 10.1104/99.104.042606.
Peace, N. (2020) Int J Environ Sci Nat Res. 23(5): 556123. DOI:10.19080/IJESNR.2020.23.556123
Pérez-Salam6 I, et al (2014). Plant Physiol. May; 165(1):319-34. doi: 10.1104/pp. 114.237891. Epub 2014 Mar. 27. PMID: 24676858; PMCID: PMC4012591.
Pick, T. et al., (2012). Plant Physiol. 159(1):52-5. doi: 10.1104/pp. 111.191841.
Quint M. et al. (2016) Nat Plants. 2:15190.
Procko C. et al (2018) Genes Dev. 30(13):1529-1541. doi:10.1101/gad.283234.116.
Reeves G. et al (2022) Nature. 602(7896):280-286. doi: 10.1038/s41586-021-04247-y
Rhoads D. M. et al (2006) Plant Physiol. 141(2):357-66. doi: 10.1104/pp. 106.079129.
Robinson E. A. et al. (2012) New Phytol. 194(2):321-336. doi: 10.1111/j.1469-8137.2012.04074.
Ross-Elliott TJ. et al., (2017) Elife. 6:e24125. doi:10.7554/eLife.24125
Ryu, H., Kim, K., Cho, H., Park, J., Choe, S., Hwang, I. (2007). Plant Cell 19: 2749-2762.
Sawchuk M G, et al. PLOS Genetics 9(2): e1003294. https://doi.org/10.1371/journal.pgen.1003294
Savaldi-Goldstein, S. et al. (2007) Nature. 466:199-202. https://doi.org/10.1038/nature05618
Sengupta, S. et al. (2012) J. Plant Biochem. Biotechnol. 21, 15-23.
Shen W. et al (2006) Plant Cell. 18(2):422-411. doi: 10.1105/tpc.105.039750.
Shulaev V. et al (2008) Physiol Plant 132(2):199-208. doi: 10.1111/j.1399-3054.2007.01025.x
Stadler R. et al (2005) Plant J. 41(2):319-31. doi: 10.1111/j.1365-313X.2004.02298.
Sun Y. et al. (2010) Dev Cell. 19(5): 765-777.
Suzuki N. et al (2005) Plant Physiol. 139(3):1313-1322. doi: 10.1104/pp. 105.070110.
Szabados, L., Savoure, A. (2010) Trends Plant. Sci. 15, 89-97.
Takahashi, N. et al. (2002) Planta. 216, 203-211.
Timm, S. et al (2021) Plants (Basel). 10(3):430. doi:10.3390/plants10030430.
Van Der Kooij T. A. et al (2012) Phyton 36(2):173-184.
Vert, G., Chory, J. (2006). Nature 441: 96-100.
Vialaret J. et al. (2014) Proteomics. 14(9), 1058-1070
Verslues P. E. et al., (2006) Plant Journal February; 45(4):523-39.
Wang Z. Y. et al., (2001) Nature. 2001 Mar. 15; 410(6826):380-3.
Wang, J., et al (2021), New Phytol. https//doi.org/10.1111/nph.17152
Wang X. (2008) Dev Cell. 15(2):220-235. doi:10.1016/j.devcel.2008.06.011.
Wang Z. Y. et al., (2001) Nature. 2001 Mar. 15; 410(6826):380-3.
Wang Z. Y. et al., (2002) Dev Cell. 2(4):505-513. doi:10.1016/s1534-5807(02)00153-3.
Weidtkamp-Peters S & Stahl Y (2017). Methods Mol Biol. 1621:163-175. doi:10.1007/978-1-4939-7063-6_16.
Winter G. et al., (2015) Front Plant Sci. 9:534. doi:10.3389/fpis.2015.00534.
Xie B. et al., (2010) Plant J. 65(11):1-14. doi:10.1111/j.1365-313X.2010.04399.x.
Yamada, K. et al (2010). J Biol Chem. 285(2):1138-46. doi: 10.1074/jbc.M109.054288.
Yin, Y., Wang, Z. Y., Mora-Garcia, S., U, J., Yoshida, S., Asami, T., Chory, J. (2002). Cell 109: 181-191
Yu X. et al. (2011) Plant J. 65(4):634-46.
Zhao Y. (2018) Annu Rev Plant Biol. 69:417-435.

1. A method for modulating a plant adaptation trait, the method comprising specifically expressing the BRL3 pathway in the phloem of said plant.

2. A method according to claim 1, wherein the BRL3 pathway is overexpressed in the phloem.

3. A method according to claim 1, wherein a trait is defined as at least one of the following: the growth physiology, the tolerance to an abiotic stress, a vascular transport and/or the defense response.

4. A method according to claim 3, wherein the plant exhibits at least one of the following features within a), b), c) and/or d):
a) the growth physiology has been modulated: the plant has an increased hypocotyl growth, an increased root growth, an increased petiole length, an altered flowering time and/or modulation of a marker gene involved in phytohormone response,
b) the tolerance of the plant to an abiotic stress has been improved: the plant has an improved root hydrotropism, an enhanced programmed cell death in root meristems under osmotic stress, a greater hypocotyl growth under heat stress, improved plant survival rates under heat stress, an improved carbon utilization and/or reduced or repressed photorespiration pathway signaling and/or a capacity to accumulate metabolites in normal conditions and under heat stress;
c) the vascular transport properties of the plant has been improved: the plant exhibits an increased accumulation of metabolites in the roots, a decreased phloem unloading in the roots and/or a modulation of a marker gene for vascular transporter,
d) the defense response of the plant has been modulated,
wherein the modulation of a trait as defined above is by comparison of the same trait in a plant wherein the BRL3 pathway has not been expressed.

5. A method according to claim 1, wherein the BRL3 pathway comprises at least one of the following genes: BRL3, KIN7, ERD14, BES1, BZR1 and/or BIN2.

6. A method according to claim 1, wherein said expression is achieved at the gene and/or at the protein level in said plant.

7. A method according to claim 1, wherein the expression in the phloem is carried out by the use of a phloem specific promoter.

8. A method according to claim 1, wherein a grafting technique is used to express or overexpress or specifically express the BRL3 pathway in the phloem of said plant.

9. A method according to claim 8, wherein a shoot scion expressing the BRL3 pathway is grafted onto a rootstock of said plant.

10. A plant obtained by a method for modulating a plant adaptation trait, the method comprising specifically expressing the BRL3 pathway in the phloem of said plant.

11. A plant preferably according to claim 10, wherein the plant comprises an expression vector comprising a nucleic acid sequence encoding a polypeptide of the BRL3 pathway, such as BRL3, KIN7, ERD14, BES1, BZR1 and/or BIN2, wherein said expression vector is suitable for specific expression in the phloem.

12. A plant according to claim 11, wherein the expression in the phloem is achieved by the presence of a phloem specific promoter.

13. A plant according to claim 11, comprising an expression vector comprising a nucleic acid sequence which has at least 50% identity with SEQ ID NO:1 and which is operably linked to a phloem specific promoter.

14. A plant according to claim 10 which is a drought tolerant plant.

15. A plant according to claim 10, wherein the plant comprises a shoot scion expressing the BRL3 pathway, wherein said shoot scion has been grafted onto a rootstock of said plant.

16. A plant according to claim 10, wherein the plant genus is selected from Arabidopsis, Solanum, Glycine, Zea, Cucumis, Phaseolus, Saccharum, Vicia, Oryza, Triticum, Persea Americana, Vitis Vinifera and Sorghum.

17. A plant according to claim 10, wherein phloem-specific expression of the BRL3 pathway decreases phloem unloading in the roots.

18. (canceled)

19. A plant according to claim 10, wherein carbon utilization has been improved and/or photorespiration pathway signaling has been reduced or repressed in said plant.

20-23. (canceled)

24. A part of the plant as defined in claim 10.

25. The part of the plant as defined in claim 24, wherein the part is a shoot scion or a root (or rootstock or root system).
